SEQUENCE LISTING (40 sequences)

SEQ ID NO: 1 - protein, 2366 aa, Clostridium difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 2 - protein, 2366 aa, Clostridium difficile
MSLVNRKQLEKMANVRFRVQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTIVESATNDTLESFRENLNDPRFDYNKFYRKRMEIIYDKQKNFINYYKTQREENPDLIIDDIVKIYLSNEYSKDIDELNSYIEESLNKVTENSGNDVRNFEEFKGGESFKLYEQELVERWNLAAASDILRISALKEVGGVYLDVDMLPGIQPDLFESIEKPSSVTVDFWEMVKLEAIMKYKEYIPGYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNAFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKKNYFEGSLGEDDNLDFSQNTVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGLDVDSLSTEIETAIDLAKEDISPKSIEINLLGCNMFSYSVNVEETYPGKLLLRVKDKVSELMPSISQDSIIVSANQYEVRINSEGRRELLDHSGEWINKEESIIKDISSKEYISFNPKENKIIVKSKNLPELSTLLQEIRNNSNSSDIELEEKVMLAECEINVISNIDTQVVEGRIEEAKSLTSDSINYIKNEFKLIESISDALYDLKQQNELEESHFISFEDILETDEGFSIRFIDKETGESIFVETEKAIFSEYANHITEEISKIKGTIFDTVNGKLVKKVNLDATHEVNTLNAAFFIQSLIEYNSSKESLSNLSVAMKVQVYAQLFSTGLNTITDAAKVVELVSTALDETIDLLPTLSEGLPVIATIIDGVSLGAAIKELSETSDPLLRQEIEAKIGIMAVNLTAATTAIITSSLGIASGFSILLVPLAGISAGIPSLVNNELILRDKATKVVDYFSHISLAESEGAFTSLDDKIMMPQDDLVISEIDFNNNSITLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPVITTEYIREKLSYSFYGSGGTYALSLSQYNMNINIELNENDTWVIDVDNVVRDVTIESDKIKKGDLIENILSKLSIEDNKIILDNHEINFSGTLNGGNGFVSLTFSILEGINAVIEVDLLSKSYKVLISGELKTLMANSNSVQQKIDYIGLNSELQKNIPYSFMDDKGKENGFINCSTKEGLFVSELSDVVLISKVYMDNSKPLFGYCSNDLKDVKVITKDDVIILTGYYLKDDIKISLSFTIQDENTIKLNGVYLDENGVAEILKFMNKKGSTNTSDSLMSFLESMNIKSIFINSLQSNTKLILDTNFIISGTTSIGQFEFICDKDNNIQPYFIKFNTLETKYTLYVGNRQNMIVEPNYDLDDSGDISSTVINFSQKYLYGIDSCVNKVIISPNIYTDEINITPIYEANNTYPEVIVLDTNYISEKINININDLSIRYVWSNDGSDFILMSTDEENKVSQVKIRFTNVFKGNTISDKISFNFSDKQDVSINKVISTFTPSYYVEGLLNYDLGLISLYNEKFYINNFGMMVSGLVYINDSLYYFKPPIKNLITGFTTIGDDKYYFNPDNGGAASVGETIIDGKNYYFSQNGVLQTGVFSTEDGFKYFAPADTLDENLEGEAIDFTGKLTIDENVYYFGDNYRAAIEWQTLDDEVYYFSTDTGRAFKGLNQIGDDKFYFNSDGIMQKGFVNINDKTFYFDDSGVMKSGYTEIDGKYFYFAENGEMQIGVFNTADGFKYFAHHDEDLGNEEGEALSYSGILNFNNKIYYFDDSFTAVVGWKDLEDGSKYYFDEDTAEAYIGISIINDGKYYFNDSGIMQIGFVTINNEVFYFSDSGIVESGMQNIDDNYFYIDENGLVQIGVFDTSDGYKYFAPANTVNDNIYGQAVEYSGLVRVGEDVYYFGETYTIETGWIYDMENESDKYYFDPETKKAYKGINVIDDIKYYFDENGIMRTGLITFEDNHYYFNEDGIMQYGYLNIEDKTFYFSEDGIMQIGVFNTPDGFKYFAHQNTLDENFEGESINYTGWLDLDEKRYYFTDEYIAATGSVIIDGEEYYFDPDTAQLVISE

SEQ ID NO: 3 - protein, 716 aa, Clostridium difficile
NRQNMIVEPNYDLDDSGDISSTVINFSQKYLYGIDSCVNKVIISPNIYTDEINITPIYEANNTYPEVIVLDTNYISEKINININDLSIRYVWSNDGSDFILMSTDEENKVSQVKIRFTNVFKGNTISDKISFNFSDKQDVSINKVISTFTPSYYVEGLLNYDLGLISLYNEKFYINNFGMMVSGLVYINDSLYYFKPPIKNLITGFTTIGDDKYYFNPDNGGAASVGETIIDGKNYYFSQNGVLQTGVFSTEDGFKYFAPADTLDENLEGEAIDFTGKLTIDENVYYFGDNYRAAIEWQTLDDEVYYFSTDTGRAFKGLNQIGDDKFYFNSDGIMQKGFVNINDKTFYFDDSGVMKSGYTEIDGKYFYFAENGEMQIGVFNTADGFKYFAHHDEDLGNEEGEALSYSGILNFNNKIYYFDDSFTAVVGWKDLEDGSKYYFDEDTAEAYIGISIINDGKYYFNDSGIMQIGFVTINNEVFYFSDSGIVESGMQNIDDNYFYIDENGLVQIGVFDTSDGYKYFAPANTVNDNIYGQAVEYSGLVRVGEDVYYFGETYTIETGWIYDMENESDKYYFDPETKKAYKGINVIDDIKYYFDENGIMRTGLITFEDNHYYFNEDGIMQYGYLNIEDKTFYFSEDGIMQIGVFNTPDGFKYFAHQNTLDENFEGESINYTGWLDLDEKRYYFTDEYIAATGSVIIDGEEYYFDPDTAQLVISE

SEQ ID NO: 4 - protein, 2710 aa, Clostridium difficile
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 5 - protein, 2710 aa, Clostridium difficile
MSLISKEELIKLAYSIRPRENEYKTILTNLDEYNKLTTNNNENKYLQLKKLNESIDVFMNKYKNSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETLLESYRTNSLRKINSNHGIDIRANSLFTEQELLNIYSQELLNRGNLAAASDIVRLLALKNFGGVYLDVDMLPGIHSDLFKTIPRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKDSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKEYGPLKTEDDKILVPIDDLVISEIDFNNNSIKLGTCNILAMEGGSGHTVTGNIDHFFSSPYISSHIPSLSVYSAIGIKTENLDFSKKIMMLPNAPSRVFWWETGAVPGLRSLENNGTKLLDSIRDLYPGKFYWRFYAFFDYAITTLKPVYEDTNTKIKLDKDTRNFIMPTITTDEIRNKLSYSFDGAGGTYSLLLSSYPISMNINLSKDDLWIFNIDNEVREISIENGTIKKGNLIEDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNGSTLEFNSKDFIAEDINVFMKDDINTITGKYYVDNNTDKSIDFSISLVSKNQVKVNGLYLNESVYSSYLDFVKNSDGHHNTSNFMNLFLNNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIESNLVTGWQTINGKKYYFDINTGAASTSYKIINGKHFYFNNNGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSGSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTSIASTGYTIINGKYFYFNTDGIMQIGVFKVPNGFEYFAPANTHNNNIEGQAILYQNKFLTLNGKKYYFGSDSKAITGWQTIDGKKYYFNPNNAIAATHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDSKKYYFNLNTAVAVTGWQTIDGEKYYFNLNTAEAATGWQTIDGKRYYFNTNTYIASTGYTIINGKHFYFNTDGIMQIGVFKGPDGFEYFAPANTHNNNIEGQAILYQNKFLTLNGKKYYFGSDSKAVTGLRTIDGKKYYFNTNTAVAVTGWQTINGKKYYFNTNTYIASTGYTIISGKHFYFNTDGIMQIGVFKGPDGFEYFAPANTDANNIEGQAIRYQNRFLYLHDNIYYFGNDSKAATGWATIDGNRYYFEPNTAMGANGYKTIDNKNFYFRNGLPQIGVFKGPNGFEYFAPANTDANNIDGQAIRYQNRFLHLLGKIYYFGNNSKAVTGWQTINSKVYYFMPDTAMAAAGGLFEIDGVIYFFGVDGVKAPGIYG

SEQ ID NO: 6 - protein, 879 aa, Clostridium difficile
GLININNSLFYFDPIESNLVTGWQTINGKKYYFDINTGAASTSYKIINGKHFYFNNNGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSGSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTSIASTGYTIINGKYFYFNTDGIMQIGVFKVPNGFEYFAPANTHNNNIEGQAILYQNKFLTLNGKKYYFGSDSKAITGWQTIDGKKYYFNPNNAIAATHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDSKKYYFNLNTAVAVTGWQTIDGEKYYFNLNTAEAATGWQTIDGKRYYFNTNTYIASTGYTIINGKHFYFNTDGIMQIGVFKGPDGFEYFAPANTHNNNIEGQAILYQNKFLTLNGKKYYFGSDSKAVTGLRTIDGKKYYFNTNTAVAVTGWQTINGKKYYFNTNTYIASTGYTIISGKHFYFNTDGIMQIGVFKGPDGFEYFAPANTDANNIEGQAIRYQNRFLYLHDNIYYFGNDSKAATGWATIDGNRYYFEPNTAMGANGYKTIDNKNFYFRNGLPQIGVFKGPNGFEYFAPANTDANNIDGQAIRYQNRFLHLLGKIYYFGNNSKAVTGWQTINSKVYYFMPDTAMAAAGGLFEIDGVIYFFGVDGVKAPGIYG

SEQ ID NO: 7 - protein, 18 aa, Clostridium difficile
SPNIYTDEINITPVYETN

SEQ ID NO: 8 - protein, 16 aa, Clostridium difficile
YPEVIVLDANYINEKI

SEQ ID NO: 9 - protein, 15 aa, Clostridium difficile
TVGDDKYYFNPINGG

SEQ ID NO: 10 - protein, 18 aa, Clostridium difficile
ASIGETIIDDKNYYFNQS

SEQ ID NO: 11 - protein, 16 aa, Clostridium difficile
EDGFKYFAPANTLDEN

SEQ ID NO: 12 - protein, 12 aa, Clostridium difficile
PANTLDENLEGE

SEQ ID NO: 13 - protein, 12 aa, Clostridium difficile
AIDFTGKLIIDE

SEQ ID NO: 14 - protein, 14 aa, Clostridium difficile
NIYYFDDNYRGAVE

SEQ ID NO: 15 - protein, 12 aa, Clostridium difficile
HYFSPETGKAFK

SEQ ID NO: 16 - protein, 13 aa, Clostridium difficile
IGDYKYFNSDGVM

SEQ ID NO: 17 - protein, 22 aa, Clostridium difficile
HFYFAENGEMQIGVFNTEDGFK

SEQ ID NO: 18 - protein, 16 aa, Clostridium difficile
INDGQYYFNDDGIMQV

SEQ ID NO: 19 - protein, 16 aa, Clostridium difficile
YKYFAPANTVNDNIYG

SEQ ID NO: 20 - protein, 14 aa, Clostridium difficile
ESDKYYFNPETKKA

SEQ ID NO: 21 - protein, 19 aa, Clostridium difficile
NNNYYFNENGEMQFGYINI

SEQ ID NO: 22 - protein, 16 aa, Clostridium difficile
QNTLDENFEGESINYT

SEQ ID NO: 23 - protein, 18 aa, Clostridium difficile
SPNIYTDEINITPIYEAN

SEQ ID NO: 24 - protein, 16 aa, Clostridium difficile
YPEVIVLDTNYISEKI

SEQ ID NO: 25 - protein, 15 aa, Clostridium difficile
TIGDDKYYFNPDNGG

SEQ ID NO: 26 - protein, 18 aa, Clostridium difficile
ASVGETIIDGKNYYFSQN

SEQ ID NO: 27 - protein, 16 aa, Clostridium difficile
EDGFKYFAPADTLDEN

SEQ ID NO: 28 - protein, 12 aa, Clostridium difficile
PADTLDENLEGE

SEQ ID NO: 29 - protein, 12 aa, Clostridium difficile
AIDFTGKLTIDE

SEQ ID NO: 30 - protein, 14 aa, Clostridium difficile
NVYYFGDNYRAAIE

SEQ ID NO: 31 - protein, 12 aa, Clostridium difficile
YYFSTDTGRAFK

SEQ ID NO: 32 - protein, 14 aa, Clostridium difficile
IGDDKFYFNSDGIM

SEQ ID NO: 33 - protein, 22 aa, Clostridium difficile
YFYFAENGEMQIGVFNTADGFK

SEQ ID NO: 34 - protein, 16 aa, Clostridium difficile
INDGKYYFNDSGIMQI

SEQ ID NO: 35 - protein, 15 aa, Clostridium difficile
YKYFAPANTVNDNIY

SEQ ID NO: 36 - protein, 14 aa, Clostridium difficile
ESDKYYFDPETKKA

SEQ ID NO: 37 - protein, 19 aa, Clostridium difficile
DNHYYFNEDGIMQYGYLNI

SEQ ID NO: 38 - protein, 16 aa, Clostridium difficile
QNTLDENFEGESINYT

SEQ ID NO: 39 - protein, 190 aa, Clostridium difficile
DDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIA

SEQ ID NO: 40 - protein, 190 aa, Clostridium difficile
DENGLVQIGVFDTSDGYKYFAPANTVNDNIYGQAVEYSGLVRVGEDVYYFGETYTIETGWIYDMENESDKYYFDPETKKAYKGINVIDDIKYYFDENGIMRTGLITFEDNHYYFNEDGIMQYGYLNIEDKTFYFSEDGIMQIGVFNTPDGFKYFAHQNTLDENFEGESINYTGWLDLDEKRYYFTDEYIA

(19) United States
(12) Patent Application Publication
(10) Pub. No.: US 2024/0148777 A1
(43) Pub. Date: May 9, 2024

(54) METHODS AND COMPOSITIONS FOR TREATING CLOSTRIDIODES DIFFICILE INFECTIONS

(71) Applicant: Matrivax, Inc., Boston, MA (US)

(72) Inventors: Kevin P. KILLEEN, Needham, MA (US); Ann THANAWASTIEN, Brookline, MA (US); Robert T. CARTEE, Kingston, MA (US); Michael W. FINN, Somerville, MA (US)

(21) Appl. No.: 18/476,600

(22) Filed: Sep. 28, 2023

Related U.S. Application Data
(63) Continuation of application No. PCT/US2022/023029, filed on Apr. 1, 2022.
(60) Provisional application No. 63/170,250, filed on Apr. 2, 2021.

(51) Int. Cl. A61K 31/715; A61K 38/16; A61K 47/02; A61K 47/26; A61P 31/04

(52) U.S. Cl. CPC A61K 31/715; A61K 38/164; A61K 47/02; A61K 47/26; A61P 31/04

(57) ABSTRACT

Provided herein are immunogenic compositions for treating Clostridioides difficile infections.

### CROSS-REFERENCE

This application is a Continuation of International Patent Application No. PCT/US2022/023029, filed Apr. 1, 2022, which claims the benefit of U.S. Provisional Patent Application No. 63/170,250, filed Apr. 2, 2021, the content of which is incorporated by reference herein in its entirety.

### SEQUENCE LISTING

The instant application contains a Sequence Listing which has been submitted electronically in XML format and is hereby incorporated by reference in its entirety. Said XML copy, created on Oct. 5, 2023, is named 51744-701_301_SL.xml and is 48,014 bytes in size.

### BACKGROUND

Clostridioides difficile (C. difficile) infection (CDI) is a symptomatic infection caused by the spore-forming bacterium, C. difficile. CDI makes up about 20% of cases of antibiotic-associated diarrhea and can cause life-threatening inflammation in the colon. Effective methods of eliciting an immune response against CDI are needed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

Disclosed herein is a composition comprising a cell-surface polysaccharide from Clostridioides difficile (C. difficile), wherein the cell-surface polysaccharide is a PSII antigen enriched from C. difficile, wherein the composition has a total carbohydrate percentage by weight of at least about 40%, and wherein the PSII antigen enriched from C. difficile is at least 90% of the total carbohydrate percentage.

Disclosed herein is a composition comprising: (a) a cell-surface polysaccharide of Clostridioides difficile (C. difficile); and (b) a first polypeptide comprising a carrier protein;

wherein the carrier protein and the cell-surface polysaccharide of C. difficile are present in the composition at a ratio of less than about 10:1 to about 1:3. Disclosed herein is a composition comprising: (a) a cell-surface polysaccharide of Clostridioides difficile (C. difficile); and (b) a first polypeptide comprising a carrier protein derived from an organism other than C. difficile; wherein the carrier protein and the cell-surface polysaccharide are present in the composition at a ratio of from about 10:1 to about 1:10.

Disclosed herein is a pharmaceutical composition comprising: (a) a cell-surface polysaccharide enriched from Clostridioides difficile (C. difficile); (b) a first polypeptide or a first polynucleotide encoding the first polypeptide, wherein the first polypeptide comprises a first toxoid of C. difficile or a fragment thereof; (c) a second polypeptide or a second polynucleotide encoding the second polypeptide, wherein the second polypeptide comprises a second toxoid of C. difficile or a fragment thereof; and (d) a pharmaceutically acceptable carrier; wherein the pharmaceutical composition has a total carbohydrate percentage, wherein at least about 90% of the total carbohydrate percentage is PSII.

Disclosed herein is a pharmaceutical composition comprising: (a) a cell-surface polysaccharide enriched from Clostridioides difficile (C. difficile); (b) a first polypeptide comprising a first toxoid of C. difficile or a fragment thereof; (c) a second polypeptide comprising a second toxoid of C. difficile or a fragment thereof; and (d) a pharmaceutically acceptable carrier; wherein the cell-surface polysaccharide enriched from C. difficile is not conjugated to the first polypeptide or the second polypeptide.

Disclosed herein is a pharmaceutical composition comprising: (a) a cell-surface polysaccharide of Clostridioides difficile (C. difficile); and (b) a first polypeptide or a polynucleotide encoding the first polypeptide, wherein the polypeptide comprises a first toxoid of C. difficile or a fragment thereof that is an inactivated toxin; wherein the pharmaceutical composition comprises less than about 5% by weight of a C. difficile protein other than an inactivated toxin of C. difficile.

Disclosed herein is a pharmaceutical composition comprising: (a) a cell-surface polysaccharide from Clostridioides difficile (C. difficile); (b) a first polypeptide or a first polynucleotide encoding the first polypeptide, wherein the first polypeptide comprises a first toxoid of C. difficile or a fragment thereof; (c) a second polypeptide or a second polynucleotide encoding the second polypeptide, wherein the second polypeptide comprises a second toxoid of C. difficile or a fragment thereof, and (d) a pharmaceutically acceptable carrier; wherein the first toxoid is a full-length toxin A toxoid and the second toxoid is a full-length toxin B toxoid.

Disclosed herein is a pharmaceutical composition comprising: (a) a cell-surface polysaccharide of Clostridioides difficile (C. difficile); and (b) a first polypeptide or a polynucleotide encoding the first polypeptide, wherein the first polypeptide comprises a first inactivated toxin of C. difficile or a fragment thereof, wherein the pharmaceutical composition comprises less than about 5% by weight of a C. difficile protein other than an inactivated toxin of C. difficile. Also disclosed herein is a pharmaceutical composition comprising a cell-surface polysaccharide of Clostridioides difficile (C. difficile) conjugated to a carrier protein; wherein the cell-surface polysaccharide of C. difficile is conjugated to the carrier protein via a chemical linker comprising from 1 carbon to 10 carbon atoms.

Disclosed herein is a pharmaceutical composition comprising: (a) a cell-surface polysaccharide from Clostridioides difficile (C. difficile) conjugated to a carrier protein; (b) a first polypeptide comprising a first toxoid of C. difficile or a fragment thereof (c) a second polypeptide comprising a second toxoid of C. difficile or a fragment thereof; and (d) a pharmaceutically acceptable carrier; wherein the cell-surface polysaccharide from C. difficile is conjugated to the carrier protein via a chemical linker comprising a thiosuccinimide or a thioester.

Disclosed herein is a pharmaceutical composition consisting essentially of: (a) a cell-surface polysaccharide from Clostridioides difficile (C. difficile) conjugated to a pharmaceutically acceptable carrier; (b) a first polypeptide or a first polynucleotide encoding the first polypeptide, wherein the first polypeptide comprises a first toxoid of C. difficile or a fragment thereof (c) a second polypeptide or a second polynucleotide encoding the second polypeptide, wherein the second polypeptide comprises a second toxoid of C. difficile or a fragment thereof and (d) an adjuvant.

Disclosed herein is a method of treating, preventing or inhibiting manifestation or symptoms an infection comprising administering a therapeutically effective amount of a composition or a pharmaceutical composition of the disclosure to a subject in need thereof.

Disclosed herein is a method of treating an infection comprising: (a) administering a therapeutically effective amount of a composition or a pharmaceutical composition of the disclosure to a subject in need thereof; wherein the infection is a Clostridioides Difficile (C. difficile) infection; (b) after the administering of the pharmaceutical composition, collecting a fecal sample from the subject; and (c) analyzing the fecal sample and determining a change in colony-forming units (CFU)/mg of C. difficile in feces a marker of infection.

Disclosed herein is a method of treating an infection, the method comprising administering to a subject in need thereof a therapeutically effective amount of a pharmaceutical composition comprising: (a) a cell-surface polysaccharide of Clostridioides Difficile (C. difficile); and (b) a polypeptide or polynucleotide encoding the polypeptide, wherein the polypeptide comprises a toxoid of C. difficile or a fragment thereof; wherein the administering is weekly or biweekly, and wherein the infection is caused by C. difficile.

Disclosed herein is a method comprising: (a) selecting a subject from a group consisting of: (i) a first subject, wherein the first subject is greater than 55 years of age; (ii) a second subject, wherein the second has a history of a C. difficile infection within a 6-month period; and (iii) a third subject, wherein the third subject has a positive C. difficile colony forming units (CFU)/mg count; and (b) administering a therapeutically effective amount of a pharmaceutical composition comprising a cell-surface polysaccharide of C. difficile; and a polypeptide or polynucleotide encoding the polypeptide, wherein the polypeptide comprises a toxoid of C. difficile or a fragment thereof.

Disclosed herein is a method of treating an infection, (a) identifying genetic sequence information of a biological sample obtained from a subject to determine a presence of the infection, wherein the infection is a Clostridioides difficile (C. difficile) infection; and (b) administering to the subject a therapeutically effective amount of a pharmaceutical composition comprising a cell-surface polysaccharide of C. difficile.

Disclosed herein is a method of treating an infection, the method comprising administering to a subject in need there of: (a) a therapeutically effective amount of a pharmaceutical composition of the disclosure; and (b) a therapeutic agent; wherein the infection is caused by Clostridioides difficile (C. difficile).

Disclosed herein is a method of treating an infection, the method comprising administering a pharmaceutical composition comprising a cell-surface polysaccharide of Clostridioides difficile (C. difficile), wherein the administering causes an immunoglobulin M (IgM) specific to the cell-surface polysaccharide to increase by at least about 250-fold compared to an absence of the pharmaceutical composition.

Disclosed herein is a method of enriching for a cell-surface polysaccharide of Clostridioides difficile (C. difficile) comprising: (a) obtaining a cell-surface extract (CSE) of one or more strains of C. difficile, and (b) enriching for a cell-surface polysaccharide of C. difficile from the CSE, thereby obtaining an enriched cell-surface polysaccharide of C. difficile sample; wherein the enriched cell-surface polysaccharide of C. difficile sample comprises less than about 5% by weight of a C. difficile impurity.

### BRIEF DESCRIPTION OF THE DRAWINGS

FIG. 1 shows the SEC MALS analysis of PSII purified by the process described and the corresponding parameters.

FIG. 2 illustrates an overview of TEMPO, CDAP, and aminooxy conjugation chemistry schemes and the resulting “lattice”- and “star”-like conjugate structures.

FIG. 3 TOP PANEL shows the SEC MALS trace for the PSII-CRM₁₉₇ conjugate synthesized with TEMPO/EDC chemistry. BOTTOM PANEL shows the analytical summary of the TEMPO/EDC/PSII-CRM₁₉₇ conjugate.

FIG. 4A shows the absorbance traces at 280 nm (A280) of CRM-maleimide and PSII-CRM₁₉₇ conjugate synthesized with CDAP chemistry separated by SEC-HPLC. FIG. 4B shows a SEC-MALS trace of the PSII-CRM₁₉₇ CDAP conjugate. FIG. 4C shows the corresponding determined MW of the components and SEC MALS parameters used to analyze the PSII-CRM₁₉₇ CDAP conjugate.

FIG. 5A shows PSII-CRM₁₉₇ conjugate synthesized with end-linked thiol chemistry and CRM-maleimide A280 traces following separation by SEC. FIG. 5B shows a SEC-MALs trace of PSII-CRM. FIG. 5C shows the analytical parameters of the SEC MALS analysis, OD280 (A280), and resorcinol assay.

FIG. 6 outlines the steps for stripping C. difficile cells using sodium deoxycholate (DOC) for PSII purification.

FIG. 7 outlines the procedure for the 20% ethanol extraction stage of PSII purification.

FIG. 8 outlines the procedure for the first TCA precipitation of the 20% ethanol extracted material for purification of PSII.

FIG. 9 outlines the procedure for ultrafiltering/diafiltering (UFDF 1) PSII after the first TCA precipitation, and addition of ammonium sulfate in preparation for the hydrophobic interaction chromatography stage on a Phenyl Sepharose column.

FIG. 10 outlines the procedure for applying the UFDF 1 material onto the Phenyl Sepharose column and collection of the flow through and wash fractions.

FIG. 11 outlines the procedure for the second UFDF (UFDF-2) step of pooled fractions from the Phenyl Sepharose column in preparation for separation by anionic exchange chromatography on a Q-Sepharose column.

FIG. 12 outlines the procedure for applying the UFDF-2 material on the Q-Sepharose column and eluting the PSII using 10 mM sodium phosphate buffer containing 300 mM NaCl (15% of buffer B).

FIG. 13 outlines the procedure for the second TCA precipitation of the Q-Sepharose column eluate.

FIG. 14 outlines the procedure for the ultrafiltration/diafiltration (UFDF-3) of the PSII following the second TCA precipitation into water.

FIG. 15 outlines the procedure for concentration the material from UFDF-3 on a 3 kDa MW cut-off spin filter.

FIG. 16 shows a general schematic for prophylactic immunization and challenge mouse model of CDI.

FIG. 17 PANEL A shows the timeline of the spore challenge, immunization schedule, and fecal collection time points of the individual fecal samples. PANEL B shows C. difficile outgrowth following therapeutic immunization, normalized to baseline CFU at Day 0 prior to immunization.

FIG. 18 shows % C. difficile outgrowth from baseline CFU at Day 0.

FIG. 19 PANEL A illustrates the immunization schedule used to obtain Anti-PSII IgG data. PANEL B shows fecal colonization (CFU/mg of feces) determined 2 weeks after the first immunization treatment on day 14.

FIG. 20 shows that prophylactic PSII-CRM₁₉₇ conjugate immunization protected mice from weight loss and symptoms when treated with PSII alone, PSII-CRM₁₉₇ conjugate; and PBS control.

FIG. 21 shows the SEC-MALS chromatogram data from the analysis of the three end-linked PSII-CRM₁₉₇ conjugates. PANEL A shows the SECS-MALS chromatogram data of the 3×PSII-CRM₁₉₇ conjugate. PANEL B shows the SECS-MALS chromatogram data of the 6×PSII-CRM₁₉₇ conjugate. PANEL C shows the SECS-MALS chromatogram data of the 10×PSII-CRM₁₉₇ conjugate. PANEL D shows the SECS-MALS chromatogram data of the 3×, 6×, and 10×PSII-CRM₁₉₇ conjugates.

FIG. 22 shows protection against CDI following immunizations of PSII-CRM₁₉₇ conjugate with and without toxoids A and B.

FIG. 23 PANEL A shows molar mass vs. volume (mL) data for the end-linked 6×PSII-CRM₁₉₇ conjugate. PANEL B compares the conjugate mass, CRM₁₉₇ mass, and PSII mass of a sample.

FIG. 24 shows a representative analysis FIG. ((6×PSII-CRM), which had a PSII-conjugate signal at 30 min, and PSII and PSII thiol peaks around 38 min

FIG. 25 shows the final SEC MALS analysis results with detailed components of CRM₁₉₇ protein and PSII across the conjugate signals and an analysis with the components combined. PANEL A shows SEC MALS analysis results with 3×PSII-CRM. PANEL B shows SEC MALS analysis results with 6×PSII-CRM. PANEL C shows 10×PSII-CRM. PANEL D shows SEC MALS analysis with all components combined.

FIG. 26 shows the geometric mean weight loss following prophylactic immunization and a spore challenge in mice treated with the PSII-CRM₁₉₇ End-linked conjugate; the PSII-CRM₁₉₇ CDAP conjugate; and vehicle.

FIG. 27 shows CDI-associated body-weight loss post-C. difficile spore challenge. PANEL A shows weight loss following CDI in mice; PANEL B shows percent body weight changes compared to spore day 0 in mice treated with 3:1 PSII:CRM₁₉₇ over days 1-14; PANEL C shows percent body weight changes compared to spore day 0 in mice treated with 6:1 PSII:CRM₁₉₇ over days 1-14; PANEL D shows percent body weight changes compared to spore day 0 in mice treated with 10:1 PSII:CRM₁₉₇ over days 1-14.

FIG. 28 illustrates the timeline for the study used to determine the effects of different adjuvants.

FIG. 29 shows the CDI geometric mean weight loss post-spore infection over the first 9 days after infection.

FIG. 30 shows the CDI geometric mean weight loss post-spore infection over the first 30 days after infection.

FIG. 31A-C show anti-TcdA reciprocal titers in groups treated with toxoids A and B (AlOH); PSII-CRM₁₉₇ End-linked+toxoids A and B+AlPO₄; PSII-CRM₁₉₇ End-linked+toxoids A and B+AlOH; PSII-CRM₁₉₇ End-linked+toxoids A and B+Advax2; and sterile saline (AlPO₄) on day 14 (FIG. 31A), day 27 (FIG. 31B), and day 41 (FIG. 31C). FIG. 31D shows anti-TxdA ELISA Titers for groups treated with PSII-CRM₁₉₇; toxoids A and B (AlOH); PSII-CRM₁₉₇ End-linked+toxoids A and B+AlPO₄; PSII-CRM₁₉₇ End-linked+toxoids A and B+AlOH; PSII-CRM₁₉₇ End-linked+toxoids A and B+Advax2; and sterile saline (AlPO₄) throughout the duration of the study.

### DETAILED DESCRIPTION OF THE INVENTION

Clostridioides difficile (C. difficile) infection (CDI) is a symptomatic infection caused by the spore-forming bacterium, C. difficile. CDI is spread by bacterial spores found within feces. Risk factors for infection include antibiotic or proton pump inhibitor use, hospitalization, or older age. Diagnosis is performed by stool culture or for testing for the bacteria's DNA or toxins. If a person tests positive for CDI without symptoms, the condition is known as C. difficile colonization. C. difficile can colonize the human colon without symptom. Approximately 2-5% of adults are carriers, and the risk of colonization has been linked to a history of unrelated diarrheal illnesses (e.g., laxative abuse and food poisoning due to Salmonellosis or Vibrio cholerae infection). Complications of CDI include pseudomembranous colitis, toxic megacolon, perforation of the colon, and sepsis. Discontinuation of antibiotics can result in resolution of symptoms within 3 days in about 20% of infected patients. Recurrences are reported in up to 25% of people.

Clostridia are anaerobic motile bacteria, ubiquitous in nature, and especially prevalent in soil. Clostridia have long and irregular (drumstick or spindle-shaped) cells with a bulge at the terminal ends. C. difficile cells are gram-positive and show optimum growth on blood agar at human body temperatures in the absence of oxygen. When stressed, the bacteria produce spores that can tolerate extreme conditions that the active bacteria cannot tolerate.

C. difficile is transmitted by the fecal-oral route. The organism forms heat-resistant spores that are not killed by alcohol-based hand cleaners or routine surface cleaning. Surfaces can become contaminated with spores, and bacterial spores can further spread through contact. The spores survive in clinical environments for long periods. Once spores are ingested, acid resistance allows the spores to pass through the stomach. Upon exposure to bile acids, the spores germinate and multiply into vegetative cells in the colon. People without a history of gastrointestinal disturbances due to antibiotic use or diarrheal illness are less likely to become colonized by C. difficile.

The use of systemic antibiotics, including broad-spectrum penicillin, cephalosporins, fluoroquinolones, and clindamycin cause the normal microbiota of the bowel to be altered. When the antibiotic kills off other bacteria in the intestine, the remaining bacteria have less competition for space and nutrients. The net effect permits more extensive growth other bacteria, such as C. difficile.

Pathogenic C. difficile strains produce multiple toxins, including enterotoxin (toxin A), cytotoxin (toxin B), and binary toxin. C. difficile exerts its effects on the gastrointestinal (GI) tract by releasing Toxins A and B, which can bind to and damage the intestinal epithelium. Both toxin A and toxin B can produce diarrhea and inflammation in infected people. Toxins A and B are glucosyltransferases that target and inactivate the Rho family of GTPases. Toxin A is associated with the secretion of fluid and generalized inflammation in the GI tract. Toxin B induces actin depolymerization by a mechanism correlated with a decrease in the ADP-ribosylation of the low molecular mass GTP-binding Rho proteins. Toxin B is considered the main determinant of virulence in recurrent CDI and is associated with more severe damage to the colon. C. difficile has a third toxin, which is a binary toxin.

Symptoms include watery diarrhea, fever, nausea, and abdominal pain. CDI can cause severe life-threatening inflammation of the colon. CDI makes up about 20% of cases of antibiotic-associated diarrhea. In adults, significant diarrhea (i.e., new onset of more than three partially formed or watery stools per 24-hour period), recent antibiotic exposure, abdominal pain, fever (up to 40.5° C.), and a distinctive odor to the stool resembling horse manure are used as clinical prediction tools for CDI. In a hospital population, prior antibiotic treatment and diarrhea or abdominal pain are used as indicators of CDI. In children, the most prevalent symptom of CDI is watery diarrhea with at least three bowel movements a day for two or more days, which can be accompanied by fever, loss of appetite, and/or abdominal pain. Patients with sever infection can develop inflammation of the colon without diarrhea.

In some embodiments, the methods and compositions of the disclosure can target C. difficile toxins and bacterial cell-surface antigens to reduce colonization and recurrence of CDI.

### Antigen

Pharmaceutical compositions of the disclosure comprise at least one antigen of C. difficile. In some embodiments, the antigen is a cell-surface polysaccharide of C. difficile. C. difficile expresses three polysaccharides, PSI, PRI, and PSIII. PSI consists of a branched pentaglycosylphosphate repeating unit. PSII is a polymer of hexaglycosylphoshpate repeating units. PSII is a conserved antigen present on the cell-surface and biofilm of C. difficile. PSII comprises a hexaglycosyl repeat structure, and is a low molecular weight polymer that is ˜10 kDa. PRI is surface-exposed, and antibodies to PSII can be detected in stool samples of patients with CDI. PSIII is a lipid-linked polysaccharide composed of diglycosylphosphate backbone with a glyceric acid sidechain and is the lipoteichoic acid of C. difficile.

In some embodiments, the cell-surface polysaccharide is a native cell-surface polysaccharide from C. difficile. In some embodiments, the cell-surface polysaccharide from C. difficile is non-synthetic. In some embodiments, the cell-surface polysaccharide from C. difficile is purified from one or more strains of C. difficile. In some embodiments, the cell-surface polysaccharide from C. difficile is from a cell-surface extract of one or more strains of C. difficile. In some embodiments, the cell-surface polysaccharide is PSII. In some embodiments, the cell-surface polysaccharide is of the formula:

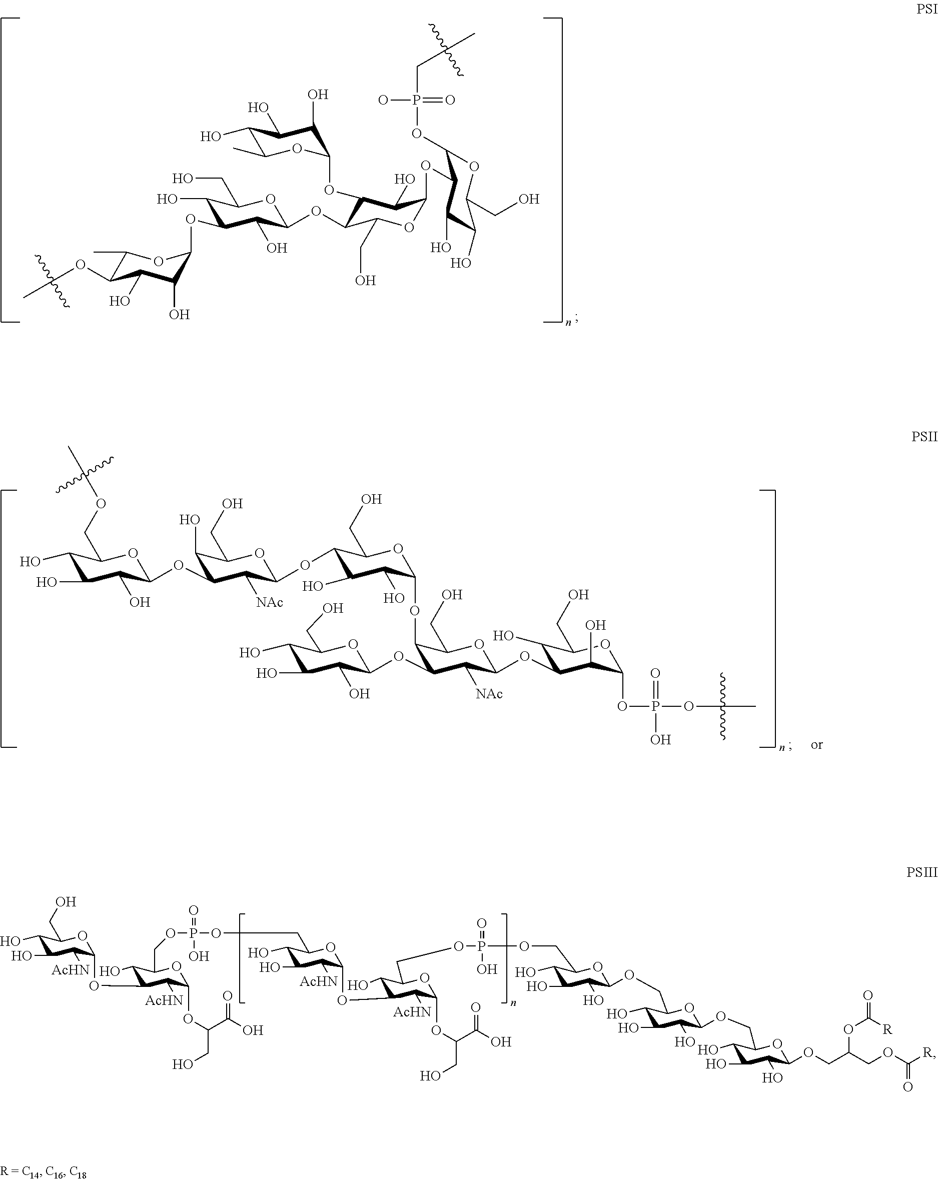

wherein n is an integer between 1 and 100, or a pharmaceutically acceptable salt thereof.

A cell-surface polysaccharide can have an average molecular weight of from about 5 kDa to about 100 kDa. In some embodiments, the cell-surface polysaccharide can have an average molecular weight of about 5 kDa to about 50 kDa. In some embodiments, the cell-surface polysaccharide can have an average molecular weight of about 5 kDa to about 25 kDa. In some embodiments, the cell-surface polysaccharide can have an average molecular weight of about 5 kDa to about 10 kDa. In some embodiments, the cell-surface polysaccharide can have an average molecular weight of about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, or about 20 kDa. In some embodiments, the cell-surface polysaccharide can have an average molecular weight of about 5 kDa. In some embodiments, the cell-surface polysaccharide can have an average molecular weight of about 8.8 kDa. In some embodiments, the cell-surface polysaccharide can have an average molecular weight of about 10 kDa.

A cell-surface polysaccharide herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

Methods of Purifying C. difficile Antigens

The C. difficile cell-surface polysaccharide antigens of the vaccine compositions of the disclosure can be obtained from inactivated cells or a cell-surface extract (CSE) from one or more C. difficile strains. In some embodiments, the polysaccharide antigens of the vaccine compositions of the disclosure can be obtained from inactivated C. difficile cells. In some embodiments, the polysaccharide antigens of the vaccine compositions of the disclosure can be obtained from a CSE of C. difficile.

In some embodiments, the C. difficile strain used to obtain purified cell-surface polysaccharide antigens is ribotype 001, 003, 027, 106, 012, 014, 036, 087, or 078. In some embodiments, the C. difficile strain can be engineered to have an increased amount of an endogenous gene product. In some embodiments, the C. difficile strain can be engineered to have an decreased amount of an endogenous gene product. In some embodiments, the C. difficile strain can be engineered to express an exogenous product.

The methods of the disclosure can obtain purified cell-surface polysaccharides from 1, 2, 3, 4, or 5 C. difficile strains. In some embodiments, the methods of the disclosure can obtain purified cell-surface polysaccharides from one C. difficile strain. In some embodiments, the methods of the disclosure can obtain purified cell-surface polysaccharides from two C. difficile strains. In some embodiments, the methods of the disclosure can obtain purified cell-surface polysaccharides from three C. difficile strains. In some embodiments, the methods of the disclosure can obtain purified cell-surface polysaccharides from four C. difficile strains. In some embodiments, the methods of the disclosure can obtain purified cell-surface polysaccharides from inactivated cells of C. difficile strain VPI 10463. In some embodiments, the methods of the disclosure can obtain purified cell-surface polysaccharides from inactivated cells of C. difficile strain BI/NAP1/027. In some embodiments, the methods of the disclosure can obtain purified cell-surface polysaccharides from inactivated cells of C. difficile strains VPI 10463 and BI/NAP1/027.

In some embodiments, disclosed herein is a method of enriching for a cell-surface polysaccharide of C. difficile comprising: (a) obtaining a cell-surface extract (CSE) of one or more strains of C. difficile, and (b) enriching for a cell-surface polysaccharide of C. difficile from the CSE, thereby obtaining an enriched cell-surface polysaccharide of C. difficile sample; wherein the enriched cell-surface polysaccharide of C. difficile sample comprises less than about 5% by weight of a C. difficile impurity. In some embodiments, the cell-surface polysaccharide of C. difficile is PSII.

In some embodiments, the method comprises: enriching for PSII from the CSE, thereby obtaining an enriched PSII sample; wherein the enriched PSII sample comprises PSII and (a) a level of peptidoglycan contamination that is less than 5% by weight peptidoglycan relative to a total weight of the PSII; (b) a level of protein contamination that is less than 5% by weight protein relative to the total weight of the PSII; or (c) a level of nucleic acid contamination that is less than 5% by weight relative to the total weight of the PSII.

In some embodiments, the obtaining comprises stripping the one or more strains of C. difficile. In some embodiments, the enriched cell-surface polysaccharide of C. difficile sample comprises less than about 5% by weight of a C. difficile protein. In some embodiments, the cell-surface polysaccharide of C. difficile is selected from the group consisting of PSI, PSII, PSIII, pharmaceutically acceptable salts thereof, and immunogenic fragments thereof. In some embodiments, the cell-surface polysaccharide of C. difficile comprises a phosphate moiety. In some embodiments, the cell-surface polysaccharide of C. difficile is PSII, a pharmaceutically acceptable salt thereof, or an immunogenic fragment thereof.

In some embodiments, the enriching comprises a step of ethanol precipitation. In some embodiments, the enriching comprises one or more steps of TCA precipitation. In some embodiments, the enriching comprises a step of ultrafiltration/diafiltration (UFDF). In some embodiments, the enriching comprises a step of ion exchange chromatography. In some embodiments, the enriching comprises one or more steps of TCA precipitation after the step of ion exchange chromatography. In some embodiments, the enriching comprises one or more steps of ultrafiltration/diafiltration (UFDF) after the step of TCA precipitation and/or after the step of ion exchange chromatography.

In some embodiments, the enriching comprises a step of filtration. In some embodiments, the step of filtration comprises tangential flow filtration or centrifugation through a filter with a molecular weight cut off. In some embodiments, the filter has a molecular weight cut off of 3 kDa or less. In some embodiments, the filter has a molecular weight cut off of 10 kDa or more. In some embodiments, the method further comprises lyophilization.

In some embodiments, the enriched PSII sample comprises PSII and a level of peptidoglycan contamination that is less than 5% by weight peptidoglycan relative to the total weight of the PSII according to NMR. In some embodiments, the enriched PSII sample comprises PSII and a level of protein contamination that is less than 5% by weight protein relative to the total weight of the PSII according to NMR. In some embodiments, the enriched PSII sample comprises PSII and a level of nucleic acid contamination that is less than 5% by weight nucleic acid relative to the total weight of the PSII according to NMR.

### PSII-Carrier Conjugates

The C. difficile cell-surface polysaccharides of the disclosure can be conjugated to a carrier molecule. In some embodiments, the carrier molecule is a protein. In some embodiments, the carrier molecule is bovine serum albumin (BSA). In some embodiments, the carrier molecule is a cross-reactive molecule, for example, CRM₁₉₇. In some embodiments, the carrier molecule is major immunoenhancing protein (MIEP). In some embodiments, the carrier molecule is diphtheria toxoid. In some embodiments, the carrier molecule is tetanus toxoid. In some embodiments, the carrier molecule is a protein derived from Bordetella. In some embodiments, the carrier molecule is a detoxified form of full-length toxin A and/or toxin B from C. difficile or a non-toxigenic fragment of toxin A and/or toxin B.

A C. difficile cell-surface polysaccharide of the disclosure can be conjugated to a carrier molecule by a chemical bond. In some embodiments, a C. difficile cell-surface polysaccharide of the disclosure can be conjugated to a carrier molecule by linking a hydroxy group of the polysaccharide to the carrier molecule. In some embodiments, a C. difficile cell-surface polysaccharide of the disclosure can be conjugated to a carrier molecule by linking primary hydroxy groups of the polysaccharide to the carrier molecule. In some embodiments, a C. difficile cell-surface polysaccharide of the disclosure can be conjugated to a carrier molecule by linking random hydroxy groups of the polysaccharide to the carrier molecule. In some embodiments, a C. difficile cell-surface polysaccharide of the disclosure can be conjugated to a carrier molecule by linking a reducing terminal of the polysaccharide to the carrier molecule. In some embodiments, a C. difficile cell-surface polysaccharide of the disclosure can be conjugated to a carrier molecule by linking a carboxyl group of the polysaccharide to the carrier molecule. In some embodiments, a C. difficile cell-surface polysaccharide can be derivatized with linkers to introduce a functional group to conjugate a carrier protein.

In some embodiments, a cell-surface polysaccharide of the disclosure can be conjugated to a hydrazide-derivatized carrier protein. In some embodiments, a cell-surface polysaccharide of the disclosure can be conjugated to a maleimide-derivatized carrier protein. In some embodiments, a cell-surface polysaccharide of the disclosure can be conjugated by derivatizing random hydroxyl groups to thiol groups; and conjugating thiolated polysaccharides with a maleimide-derivatized carrier protein. In some embodiments, a cell-surface polysaccharide of the disclosure can be conjugated to a carrier protein by oxidizing primary hydryl groups into carboxyl groups; and conjugating carboxylated polysaccharides with a hydrazide-derivatized carrier protein. In some embodiments, a cell-surface polysaccharide of the disclosure can be conjugated to a carrier protein by thiolating a reducing terminal; and conjugating the thiolated polysaccharide with a maleimide-derivatize carrier protein.

In some embodiments, a carrier protein can be conjugated to more than one cell-surface polysaccharide. In some embodiments, a carrier protein can be conjugated to more than one cell-surface polysaccharide at random locations throughout the cell-surface polysaccharide to form a lattice-like structure. In some embodiments, a carrier protein can be conjugated to more than one cell-surface polysaccharide at positions at the locations of primary hydroxyl groups throughout the cell-surface polysaccharide to form a lattice-like structure. In some embodiments, a carrier protein can be conjugated to more than one cell-surface polysaccharide at a reducing end of each cell-surface polysaccharide moiety to form a star-like structure (e.g., one carrier protein in the center with multiple polysaccharide chains protruding outward).

In some embodiments, a carrier protein and cell-surface polysaccharide can be conjugated with a carrier protein:cell-surface polysaccharide ratio of at from about 10:1 to about 5:1, from about 5:1 to about 1:1, from about 1:1 to about 1:5, from about 1:5 to about 1:10. In some embodiments, a carrier protein and cell-surface polysaccharide can be conjugated with a carrier protein:cell-surface polysaccharide ratio of at from about 8:1 to about 5:1. In some embodiments, a carrier protein and cell-surface polysaccharide can be conjugated with a carrier protein:cell-surface polysaccharide ratio of at from about 5:1 to about 3:1. In some embodiments, a carrier protein and cell-surface polysaccharide can be conjugated with a carrier protein:cell-surface polysaccharide ratio of at from about 1:5 to about 1:7. In some embodiments, a carrier protein and cell-surface polysaccharide can be conjugated with a carrier protein:cell-surface polysaccharide ratio of at from about 1:8 to about 1:10.

In some embodiments, a carrier protein and cell-surface polysaccharide can be conjugated with a carrier protein:cell-surface polysaccharide ratio of at about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10.

In some embodiments, a carrier protein and cell-surface polysaccharide can be conjugated with a carrier protein:cell-surface polysaccharide ratio of at about 10:1. In some embodiments, a carrier protein and cell-surface polysaccharide can be conjugated with a carrier protein:cell-surface polysaccharide ratio of at about 5:1. In some embodiments, a carrier protein and cell-surface polysaccharide can be conjugated with a carrier protein:cell-surface polysaccharide ratio of at about 3:1. In some embodiments, a carrier protein and cell-surface polysaccharide can be conjugated with a carrier protein:cell-surface polysaccharide ratio of at about 1:1. In some embodiments, a carrier protein and cell-surface polysaccharide can be conjugated with a carrier protein:cell-surface polysaccharide ratio of at about 1:3. In some embodiments, a carrier protein and cell-surface polysaccharide can be conjugated with a carrier protein:cell-surface polysaccharide ratio of at about 1:4. In some embodiments, a carrier protein and cell-surface polysaccharide can be conjugated with a carrier protein:cell-surface polysaccharide ratio of at about 1:5. In some embodiments, a carrier protein and cell-surface polysaccharide can be conjugated with a carrier protein:cell-surface polysaccharide ratio of at about 1:6. In some embodiments, a carrier protein and cell-surface polysaccharide can be conjugated with a carrier protein:cell-surface polysaccharide ratio of at about 1:7. In some embodiments, a carrier protein and cell-surface polysaccharide can be conjugated with a carrier protein:cell-surface polysaccharide ratio of at about 1:8. In some embodiments, a carrier protein and cell-surface polysaccharide can be conjugated with a carrier protein:cell-surface polysaccharide ratio of at about 1:9. In some embodiments, a carrier protein and cell-surface polysaccharide can be conjugated with a carrier protein:cell-surface polysaccharide ratio of at about 1:10.

### Toxins

Pathogenic C. difficile strains produce multiple toxins, including enterotoxin (toxin A; TcdA) and cytotoxin (toxin B; TcdB). Both toxin A and toxin B can produce diarrhea and inflammation in infected people. Toxins A and B are glucosyltransferases that target and inactivate the Rho family of GTPases. Toxin B induces actin depolymerization by a mechanism correlated with a decrease in the ADP-ribosylation of the low molecular mass GTP-binding Rho proteins. C. difficile has a third toxin, which is a binary toxin.

Toxin A, also known as TcdA, is associated with the secretion of fluid and generalized inflammation in the GI tract. Toxin B, also known as TcdB, is considered the main determinant of virulence in recurrent CDI and is associated with more severe damage to the colon. Toxin B has two isoforms: toxin B from historical or non-hypervirulent strains (Toxin B_{HIST}), such as VPI 10463 (TcdB1); and toxin B from hypervirulent strains (Toxin B_{HV}), such as BI/NAP1/027 (TcdB2). In hypervirulent strains, Toxin B_{HV} is approximately 10-fold more cytotoxic in cell-based assays and at least 4-fold more lethal in a murine toxin challenge model than Toxin B_{HIST}. The increased toxicity of Toxin B_{HV} is due to differences in the amino acid sequence of the C-terminal region (i.e., in the span of amino acids from position 1651 to the C-terminal position 2366).

TABLE 1 shows a list of C. difficile strains by ribotypes and indicates which toxins are expressed.

TABLE 1

| Ribotype | Toxin A/B | Binary Toxin |
|---|---|---|
| 001 | +/+ | − |
| 002 | +/+ | − |
| 003 | +/+ | − |
| 005 | +/+ | − |
| 009 | −/− | − |
| 010 | −/− | − |
| 011 | +/+ | − |
| 012 | +/+ | − |
| 013 | +/+ | − |
| 014 | +/+ | − |
| 015 | +/+ | |
| 017 | −/+ | − |
| 018 | +/+ | − |
| 020 | +/+ | − |
| 023 | +/+ | + |
| 027 | +/+ | + |
| 029 | +/+ | − |
| 033 | −/− | + |
| 035 | −/− | − |
| 038 | −/− | |
| 039 | −/− | − |
| 045 | +/+ | − |
| 046 | +/+ | − |
| 049 | +/+ | − |
| 053/163 | +/+ | − |
| 072 | +/+ | − |
| 075 | +/+ | + |
| 078/126 | +/+ | − |
| 085 | −/− | − |
| 087 | +/+ | − |
| 104 | +/+ | |
| 106 | +/+ | |
| 128 | −/− | − |
| 150 | +/+ | − |
| 161 | +/+ | − |
| 162 | +/+ | − |
| 164 | −/− | − |
| 165 | +/+ | − |
| 166 | −/− | − |

The toxins used in the vaccine compositions of the disclosure can elicit production of antibodies that neutralize Toxin A or Toxin B in an immunized subject. The vaccine compositions of the disclosure comprise at least one polypeptide comprising a toxoid or a non-toxic immunogenic polypeptide fragment of a C. difficile toxin, which can elicit production of protective antibodies that bind to at least one C. difficile toxin. In some embodiments, a vaccine composition of the disclosure comprises Toxin A or a non-toxic, immunogenic fragment thereof. In some embodiments, a vaccine composition of the disclosure comprises Toxin B or a non-toxic, immunogenic fragment thereof. In some embodiments, a vaccine composition of the disclosure comprises Toxin A or a non-toxic, immunogenic fragment of Toxin A; and Toxin B or a non-toxic, immunogenic fragment of Toxin B. In some embodiments, a vaccine composition of the disclosure can elicit production of protective antibodies that bind to C. difficile Toxin A. In some embodiments, a vaccine composition of the disclosure can elicit production of protective antibodies that bind to C. difficile Toxin B. In some embodiments, a vaccine composition of the disclosure can elicit production of protective antibodies that bind to C. difficile Toxin A and Toxin B.

In some embodiments, the C. difficile toxin polypeptide can be a full-length Toxin A or full-length Toxin B that is non-toxic but retains immunogenic properties sufficient to induce an antibody response in a subject. In some embodiments, a C. difficile toxin polypeptide of the disclosure can be treated (e.g., formaldehyde treatment) to obtain a non-toxic form of the toxin polypeptide.

In some embodiments, the polypeptide of the vaccine composition can be a full-length C. difficile Toxin A or a full-length C. difficile Toxin B. In some embodiments, the polypeptide of the vaccine composition can be a detoxified form of the full-length C. difficile Toxin A or a full-length C. difficile Toxin B. In some embodiments, the polypeptide of the vaccine composition can be a full-length C. difficile Toxin A or a detoxified form of the full-length C. difficile Toxin B. In some embodiments, the polypeptide of the vaccine composition can be a detoxified form of the full-length C. difficile Toxin A or a detoxified form of the full-length C. difficile Toxin B. In some embodiments, a vaccine composition can comprise a full length C. difficile Toxin A and a full-length C. difficile Toxin B. In some embodiments, a full-length C. difficile Toxin A or full-length C. difficile Toxin B can be treated with a chemical to produce a non-toxic fragment of Toxin A or Toxin B that retains immunogenicity. In some embodiments, a full-length C. difficile Toxin A or full-length C. difficile Toxin B can be treated with formaldehyde to produce a non-toxic fragment of Toxin A or Toxin B that retains immunogenicity. In some embodiments, the non-toxic fragment of Toxin A or Toxin B is a fragment lacking the glucosyltransferase activity encoded at the N-terminal of the Toxin.

In some embodiments, the polypeptides are at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identical to Toxin A or Toxin B, for example SEQ ID NO: 1-40.

In some embodiments, the polypeptides are prepared from the C-terminal domain of C. difficile Toxin B, which contains combined repetitive oligopeptides (CROPs). In some embodiments, the C. difficile polypeptide comprises TcdA₁₆₄₉₋₂₇₁₀ of Toxin A. In some embodiments, the polypeptide is at least about 75% identical to any one of SEQ ID NO: 4-6. In some embodiments, the polypeptide is at least about 85% identical to any one of SEQ ID NO: 4-6. In some embodiments, the polypeptide is at least about 90% identical to any one of SEQ ID NO: 4-6. In some embodiments, the polypeptide is at least about 95% identical to any one of SEQ ID NO: 4-6. In some embodiments, the polypeptide is at least about 98% identical to any one of SEQ ID NO: 4-6.

In some embodiments, the polypeptide comprises all of the C-terminal 716 amino acids of C. difficile Toxin B. In some embodiments, the polypeptide comprises a portion of the C-terminal 716 amino acids of C. difficile Toxin B. In some embodiments, the polypeptide comprises TcdB₁₆₅₁₋₂₃₆₆ of a hypervirulent BI/NAP1/027 strain of C. difficile. In some embodiments, the polypeptide comprises the CROP region from TcdB₁₈₃₄₋₂₃₆₆. In some embodiments, the polypeptide comprises the CROP region from TcdA₁₈₃₂₋₂₇₁₀.

In some embodiments, a non-toxic polypeptide fragment comprises a portion of Toxin B that is an epitope for an anti-toxin B antibody. In some embodiments, the fragment comprising the epitope for an anti-toxin B antibody is any one of SEQ ID NO: 7-38. In some embodiments, the non-toxic polypeptide comprises amino acids 2152-2341 of Toxin B from the 10463 strain (SEQ ID NO: 39). In some embodiments, the non-toxic polypeptide fragment comprises amino acids 2152-2341 of Toxin B from the BI/NAP1/027 strain (SEQ ID NO: 40). In some embodiments, the polypeptide comprises a TcdB fragment having at least about 50%, at least about 55%, at least about 60%, at least about 65%, at least about 70%, at least about 75%, at least about 80%, at least about 85%, at least about 90%, or at least about 90% identity to SEQ ID NO: 1-3 or 7-40. In some embodiments, the polypeptide comprises a TcdB fragment having at least about 75% identity to SEQ ID NO: 1-3 or 7-40. In some embodiments, the polypeptide comprises a TcdB fragment having at least about 80% identity to SEQ ID NO: 1-3 or 7-40. In some embodiments, the polypeptide comprises a TcdB fragment having at least about 90% identity to SEQ ID NO: 1-3 or 7-40. In some embodiments, the polypeptide is at least about 95% identical to any one of SEQ ID NO: 1-3 or 7-40. In some embodiments, the polypeptide is at least about 98% identical to any one of SEQ ID NO: 1-3 or 7-40. In some embodiments, the non-toxic polypeptide comprising TcdA, TcdB, or a fragment thereof is detoxified by mutation of one or more of a glucosyltransferase, serine protease, or delivery domain.

In some embodiments, the non-toxic polypeptide fragment of C. difficile Toxin A binds to actoxumab. In some embodiments, the non-toxic polypeptide fragment of C. difficile Toxin A binds to actoxumab at two sites within the CROP domain of Toxin A: amino acids 2162-2189 and 2410-2437. In some embodiments, the non-toxic polypeptide fragment of C. difficile Toxin B binds to bezlotoxumab. In some embodiments, the non-toxic polypeptide fragment of C. difficile Toxin B binds to bezlotoxumab at two sites of the C. difficile strain VPI 10463 TcdB CROP domain spanning two CROP units: E1 (discontinuous epitope within amino acids 1806-1961), and E2 (discontinuous epitope within amino acids 2007-2093).

In some embodiments, the C. difficile non-toxic polypeptide fragment of the disclosure can be obtained from one or more strains selected from the group consisting of C. difficile ribotypes 001, 003, 027, 106, 012, 014, 036, 087, or 078. In some embodiments, the C. difficile toxin polypeptide is from a hypervirulent strain of C. difficile. In some embodiments, the C. difficile polypeptide fragment is from Toxin B of a C. difficile BI/NAP1/027 strain. In some embodiments, the non-toxic fragment of Toxin B has the amino acid sequence of SEQ ID NO: 3.

TABLE 2 lists the amino acid sequences for SEQ ID NO: 1-40.

TABLE 2

| SEQ ID NO: | Sequence |
|---|---|
| 1ª | MSLVNRKQLE KMANVRFRTQ EDEYVAILDA LEEYHNMSEN TVVEKYLKLK |
| | DINSLTDIYI DTYKKSGRNK ALKKFKEYLV TEVLELKNNN LTPVEKNLHF |
| | VWIGGQINDT AINYINQWKD VNSDYNVNVF YDSNAFLINT LKKTVVESAI |
| | NDTLESFREN LNDPRFDYNK FFRKRMEIIY DKQKNFINYY KAQREENPEL |
| | IIDDIVKTYL SNEYSKEIDE LNTYIEESLN KITQNSGNDV RNFEEFKNGE |
| | SFNLYEQELV ERWNLAAASD ILRISALKEI GGMYLDVDML PGIQPDLFES |
| | IEKPSSVTVD FWEMTKLEAI MKYKEYIPEY TSEHFDMLDE EVQSSFESVL |
| | ASKSDKSEIF SSLGDMEASP LEVKIAFNSK GIINQGLISV KDSYCSNLIV |
| | KQIENRYKIL NNSLNPAISE DNDFNTTTNT FIDSIMAEAN ADNGRFMMEL |
| | GKYLRVGFFP DVKTTINLSG PEAYAAAYQD LLMFKEGSMN IHLIEADLRN |
| | FEISKTNISQ STEQEMASLW SFDDARAKAQ FEEYKRNYFE GSLGEDDNLD |
| | FSQNIVVDKE YLLEKISSLA RSSERGYIHY IVQLQGDKIS YEAACNLFAK |
| | TPYDSVLFQK NIEDSEIAYY YNPGDGEIQE IDKYKIPSII SDRPKIKLTF |
| | IGHGKDEFNT DIFAGFDVDS LSTEIEAAID LAKEDISPKS IEINLLGCNM |
| | FSYSINVEET YPGKLLLKVK DKISELMPSI SQDSIIVSAN QYEVRINSEG |
| | RRELLDHSGE WINKEESIIK DISSKEYISF NPKENKITVK SKNLPELSTL |
| | LQEIRNNSNS SDIELEEKVM LTECEINVIS NIDTQIVEER IEEAKNLTSD |
| | SINYIKDEFK LIESISDALC DLKQQNELED SHFISFEDIS ETDEGFSIRF |
| | INKETGESIF VETEKTIFSE YANHITEEIS KIKGTIFDTV NGKLVKKVNL |
| | DTTHEVNTLN AAFFIQSLIE YNSSKESLSN LSVAMKVQVY AQLFSTGLNT |
| | ITDAAKVVEL VSTALDETID LLPTLSEGLP IIATIIDGVS LGAAIKELSE |
| | TSDPLLRQEI EAKIGIMAVN LTTATTAIIT SSLGIASGES ILLVPLAGIS |
| | AGIPSLVNNE LVLRDKATKV VDYFKHVSLV ETEGVFTLLD DKIMMPQDDL |
| | VISEIDFNNN SIVLGKCEIW RMEGGSGHTV TDDIDHFFSA PSITYREPHL |
| | SIYDVLEVQK EELDLSKDLM VLPNAPNRVF AWETGWTPGL RSLENDGTKL |
| | LDRIRDNYEG EFYWRYFAFI ADALITTLKP RYEDTNIRIN LDSNTRSFIV |
| | PIITTEYIRE KLSYSFYGSG GTYALSLSQY NMGINIELSE SDVWIIDVDN |
| | VVRDVTIESD KIKKGDLIEG ILSTLSIEEN KIILNSHEIN FSGEVNGSNG |
| | FVSLTFSILE GINAIIEVDL LSKSYKLLIS GELKILMLNS NHIQQKIDYI |
| | GFNSELQKNI PYSFVDSEGK ENGFINGSTK EGLFVSELPD VVLISKVYMD |
| | DSKPSFGYYS NNLKDVKVIT KDNVNILTGY YLKDDIKISL SLTLQDEKTI |
| | KLNSVHLDES GVAEILKFMN RKGNTNTSDS LMSFLESMNI KSIFVNFLQS |
| | NIKFILDANF IISGTTSIGQ FEFICDENDN IQPYFIKFNT LETNYTLYVG |
| | NRQNMIVEPN YDLDDSGDIS STVINFSQKY LYGIDSCVNK VVISPNIYTD |
| | EINITPVYET NNTYPEVIVL DANYINEKIN VNINDLSIRY VWSNDGNDFI |
| | LMSTSEENKV SQVKIRFVNV FKDKTLANKL SFNFSDKQDV PVSEIILSFT |
| | PSYYEDGLIG YDLGLVSLYN EKFYINNFGM MVSGLIYIND SLYYFKPPVN |
| | NLITGFVTVG DDKYYFNPIN GGAASIGETI IDDKNYYFNQ SGVLQTGVFS |
| | TEDGFKYFAP ANTLDENLEG EAIDFTGKLI IDENIYYFDD NYRGAVEWKE |
| | LDGEMHYFSP ETGKAFKGLN QIGDYKYYEN SDGVMQKGFV SINDNKHYFD |
| | DSGVMKVGYT EIDGKHFYFA ENGEMQIGVF NTEDGFKYFA HHNEDLGNEE |
| | GEEISYSGIL NFNNKIYYFD DSFTAVVGWK DLEDGSKYYF DEDTAEAYIG |
| | LSLINDGQYY FNDDGIMQVG FVTINDKVFY FSDSGIIESG VQNIDDNYFY |
| | IDDNGIVQIG VFDTSDGYKY FAPANTVNDN IYGQAVEYSG LVRVGEDVYY |
| | FGETYTIETG WIYDMENESD KYYFNPETKK ACKGINLIDD IKYYFDEKGI |
| | MRTGLISFEN NNYYFNENGE MQFGYINIED KMFYFGEDGV MQIGVFNTPD |
| | GFKYFAHQNT LDENFEGESI NYTGWLDLDE KRYYFTDEYI AATGSVIIDG |
| | EEYYFDPDTA QLVISE |
| 2^{b} | MSLVNRKQLE KMANVRFRVQ EDEYVAILDA LEEYHNMSEN TVVEKYLKLK |
| | DINSLTDIYI DTYKKSGRNK ALKKFKEYLV TEVLELKNNN LTPVEKNLHF |
| | VWIGGQINDT AINYINQWKD VNSDYNVNVF YDSNAFLINT LKKTIVESAT |
| | NDTLESFREN LNDPRFDYNK FYRKRMEIIY DKQKNFINYY KTQREENPDL |
| | IIDDIVKIYL SNEYSKDIDE LNSYIEESLN KVTENSGNDV RNFEEFKGGE |
| | SFKLYEQELV ERWNLAAASD ILRISALKEV GGVYLDVDML PGIQPDLFES |
| | IEKPSSVTVD FWEMVKLEAI MKYKEYIPGY TSEHFDMLDE EVQSSFESVL |
| | ASKSDKSEIF SSLGDMEASP LEVKIAFNSK GIINQGLISV KDSYCSNLIV |
| | KQIENRYKIL NNSLNPAISE DNDFNTTTNA FIDSIMAEAN ADNGRFMMEL |
| | GKYLRVGFFP DVKTTINLSG PEAYAAAYQD LLMFKEGSMN IHLIEADLRN |
| | FEISKTNISQ STEQEMASLW SFDDARAKAQ FEEYKKNYFE GSLGEDDNLD |
| | FSQNTVVDKE YLLEKISSLA RSSERGYIHY IVQLQGDKIS YEAACNLFAK |
| | TPYDSVLFQK NIEDSEIAYY YNPGDGEIQE IDKYKIPSII SDRPKIKLTF |
| | IGHGKDEFNT DIFAGLDVDS LSTEIETAID LAKEDISPKS IEINLLGCNM |
| | FSYSVNVEET YPGKLLLRVK DKVSELMPSI SQDSIIVSAN QYEVRINSEG |
| | RRELLDHSGE WINKEESIIK DISSKEYISF NPKENKIIVK SKNLPELSTL |
| | LQEIRNNSNS SDIELEEKVM LAECEINVIS NIDTQVVEGR IEEAKSLTSD |
| | SINYIKNEFK LIESISDALY DLKQQNELEE SHFISFEDIL ETDEGFSIRF |
| | IDKETGESIF VETEKAIFSE YANHITEEIS KIKGTIFDTV NGKLVKKVNL |
| | DATHEVNTLN AAFFIQSLIE YNSSKESLSN LSVAMKVQVY AQLFSTGLNT |
| | ITDAAKVVEL VSTALDETID LLPTLSEGLP VIATIIDGVS LGAAIKELSE |
| | TSDPLLRQEI EAKIGIMAVN LTAATTAIIT SSLGIASGES ILLVPLAGIS |
| | AGIPSLVNNE LILRDKATKV VDYFSHISLA ESEGAFTSLD DKIMMPQDDL |
| | VISEIDFNNN SITLGKCEIW RMEGGSGHTV TDDIDHFFSA PSITYREPHL |
| | SIYDVLEVQK EELDLSKDLM VLPNAPNRVF AWETGWTPGL RSLENDGTKL |
| | LDRIRDNYEG EFYWRYFAFI ADALITTLKP RYEDTNIRIN LDSNTRSFIV |
| | PVITTEYIRE KLSYSFYGSG GTYALSLSQY NMNINIELNE NDTWVIDVDN |
| | VVRDVTIESD KIKKGDLIEN ILSKLSIEDN KIILDNHEIN FSGTLNGGNG |
| | FVSLTFSILE GINAVIEVDL LSKSYKVLIS GELKTLMANS NSVQQKIDYI |
| | GLNSELQKNI PYSFMDDKGK ENGFINCSTK EGLFVSELSD VVLISKVYMD |
| | NSKPLFGYCS NDLKDVKVIT KDDVIILTGY YLKDDIKISL SFTIQDENTI |
| | KLNGVYLDEN GVAEILKFMN KKGSTNTSDS LMSFLESMNI KSIFINSLQS |
| | NTKLILDTNF IISGTTSIGQ FEFICDKDNN IQPYFIKFNT LETKYTLYVG |
| | NRQNMIVEPN YDLDDSGDIS STVINFSQKY LYGIDSCVNK VIISPNIYTD |
| | EINITPIYEA NNTYPEVIVL DTNYISEKIN ININDLSIRY VWSNDGSDFI |
| | LMSTDEENKV SQVKIRFTNV FKGNTISDKI SFNFSDKQDV SINKVISTFT |
| | PSYYVEGLLN YDLGLISLYN EKFYINNFGM MVSGLVYIND SLYYFKPPIK |
| | NLITGFTTIG DDKYYFNPDN GGAASVGETI IDGKNYYFSQ NGVLQTGVES |
| | TEDGFKYFAP ADTLDENLEG EAIDFTGKLT IDENVYYFGD NYRAAIEWQT |
| | LDDEVYYFST DTGRAFKGLN QIGDDKFYFN SDGIMQKGFV NINDKTFYFD |
| | DSGVMKSGYT EIDGKYFYFA ENGEMQIGVF NTADGEKYFA HHDEDLGNEE |
| | GEALSYSGIL NFNNKIYYFD DSFTAVVGWK DLEDGSKYYF DEDTAEAYIG |
| | ISIINDGKYY FNDSGIMQIG FVTINNEVFY FSDSGIVESG MQNIDDNYFY |
| | IDENGLVQIG VFDTSDGYKY FAPANTVNDN TYGQAVEYSG LVRVGEDVYY |
| | FGETYTIETG WIYDMENESD KYYFDPETKK AYKGINVIDD IKYYFDENGI |
| | MRTGLITFED NHYYFNEDGI MQYGYLNIED KTFYFSEDGI MQIGVFNTPD |
| | GFKYFAHQNT LDENFEGESI NYTGWLDLDE KRYYFTDEYI AATGSVIIDG |
| | EEYYFDPDTA QLVISE |
| 3^{c} | NRQNMIVEPN YDLDDSGDIS STVINFSQKY LYGIDSCVNK VIISPNIYTD |
| | EINITPIYEA NNTYPEVIVL DTNYISEKIN ININDLSIRY VWSNDGSDFI |
| | LMSTDEENKV SQVKIRFTNV FKGNTISDKI SFNFSDKQDV SINKVISTFT |
| | PSYYVEGLLN YDLGLISLYN EKFYINNFGM MVSGLVYIND SLYYFKPPIK |
| | NLITGFTTIG DDKYYFNPDN GGAASVGETI IDGKNYYFSQ NGVLQTGVFS |
| | TEDGFKYFAP ADTLDENLEG EAIDFTGKLT IDENVYYFGD NYRAAIEWOT |
| | LDDEVYYFST DTGRAFKGLN QIGDDKFYFN SDGIMQKGFV NINDKTFYFD |
| | DSGVMKSGYT EIDGKYFYFA ENGEMQIGVF NTADGFKYFA HHDEDLGNEE |
| | GEALSYSGIL NFNNKIYYFD DSFTAVVGWK DLEDGSKYYF DEDTAEAYIG |
| | ISIINDGKYY FNDSGIMQIG FVTINNEVFY FSDSGIVESG MQNIDDNYFY |
| | IDENGLVQIG VFDTSDGYKY FAPANTVNDN IYGQAVEYSG LVRVGEDVYY |
| | FGETYTIETG WIYDMENESD KYYFDPETKK AYKGINVIDD IKYYFDENGI |
| | MRTGLITFED NHYYFNEDGI MQYGYLNIED KTFYFSEDGI MQIGVFNTPD |
| | GFKYFAHQNT LDENFEGESI NYTGWLDLDE KRYYFTDEYI AATGSVIIDG |
| | EEYYFDPDTA QLVISE |
| 4^{d} | MSLISKEELI KLAYSIRPRE NEYKTILTNL DEYNKLTTNN NENKYLQLKK |
| | LNESIDVFMN KYKTSSRNRA LSNLKKDILK EVILIKNSNT SPVEKNLHFV |
| | WIGGEVSDIA LEYIKOWADI NAEYNIKLWY DSEAFLVNTL KKAIVESSTT |
| | EALQLLEEEI QNPQFDNMKF YKKRMEFIYD RQKRFINYYK SQINKPTVPT |
| | IDDIIKSHLV SEYNRDETVL ESYRTNSLRK INSNHGIDIR ANSLFTEQEL |
| | LNIYSQELLN RGNLAAASDI VRLLALKNFG GVYLDVDMLP GIHSDLFKTI |
| | SRPSSIGLDR WEMIKLEAIM KYKKYINNYT SENFDKLDQQ LKDNFKLIIE |
| | SKSEKSEIFS KLENLNVSDL EIKIAFALGS VINQALISKQ GSYLTNLVIE |
| | QVKNRYQFLN QHLNPAIESD NNFTDTTKIF HDSLFNSATA ENSMFLTKIA |
| | PYLQVGEMPE ARSTISLSGP GAYASAYYDE INLQENTIEK TLKASDLIEF |
| | KFPENNLSQL TEQEINSLWS FDQASAKYQF EKYVRDYTGG SLSEDNGVDF |
| | NKNTALDKNY LLNNKIPSNN VEEAGSKNYV HYIIQLQGDD ISYEATCNLF |
| | SKNPKNSIII QRNMNESAKS YFLSDDGESI LELNKYRIPE RLKNKEKVKV |
| | TFIGHGKDEF NTSEFARLSV DSLSNEISSF LDTIKLDISP KNVEVNLLGC |
| | NMFSYDFNVE ETYPGKLLLS IMDKITSTLP DVNKNSITIG ANQYEVRINS |
| | EGRKELLAHS GKWINKEEAI MSDLSSKEYI FFDSIDNKLK AKSKNIPGLA |
| | SISEDIKTLL LDASVSPDTK FILNNLKLNI ESSIGDYIYY EKLEPVKNII |
| | HNSIDDLIDE FNLLENVSDE LYELKKLNNL DEKYLISFED ISKNNSTYSV |
| | RFINKSNGES VYVETEKEIF SKYSEHITKE ISTIKNSIIT DVNGNLLDNI |
| | QLDHTSQVNT LNAAFFIQSL IDYSSNKDVL NDLSTSVKVQ LYAQLFSTGL |
| | NTIYDSIQLV NLISNAVNDT INVLPTITEG IPIVSTILDG INLGAAIKEL |
| | LDEHDPLLKK ELEAKVGVLA INMSLSIAAT VASIVGIGAE VTIFLLPIAG |
| | ISAGIPSLVN NELILHDKAT SVVNYFNHLS ESKKYGPLKT EDDKILVPID |
| | DLVISEIDFN NNSIKLGTCN ILAMEGGSGH TVIGNIDHFF SSPSISSHIP |
| | SLSIYSAIGI ETENLDFSKK IMMLPNAPSR VFWWETGAVP GLRSLENDGT |
| | RLLDSIRDLY PGKFYWRFYA FFDYAITTLK PVYEDTNIKI KLDKDTRNFI |
| | MPTITTNEIR NKLSYSFDGA GGTYSLLLSS YPISTNINLS KDDLWIFNID |
| | NEVREISIEN GTIKKGKLIK DVLSKIDINK NKLIIGNQTI DFSGDIDNKD |
| | RYIFLTCELD DKISLIIEIN LVAKSYSLLL SGDKNYLISN LSNTIEKINT |
| | LGLDSKNIAY NYTDESNNKY FGAISKTSQK SIIHYKKDSK NILEFYNDST |
| | LEFNSKDFIA EDINVFMKDD INTITGKYYV DNNTDKSIDF SISLVSKNQV |
| | KVNGLYLNES VYSSYLDFVK NSDGHHNTSN FMNLFLDNIS FWKLFGFENI |
| | NFVIDKYFTL VGKTNLGYVE FICDNNKNID IYFGEWKTSS SKSTIFSGNG |
| | RNVVVEPIYN PDTGEDISTS LDFSYEPLYG IDRYINKVLI APDLYTSLIN |
| | INTNYYSNEY YPEIIVLNPN TFHKKVNINL DSSSFEYKWS TEGSDFILVR |
| | YLEESNKKIL QKIRIKGILS NTQSENKMSI DFKDIKKLSL GYIMSNFKSF |
| | NSENELDRDH LGFKIIDNKT YYYDEDSKLV KGLININNSL FYFDPIEFNL |
| | VTGWQTINGK KYYFDINTGA ALTSYKIING KHFYFNNDGV MQLGVFKGPD |
| | GFEYFAPANT QNNNIEGQAI VYQSKFLTLN GKKYYFDNNS KAVTGWRIIN |
| | NEKYYFNPNN AIAAVGLQVI DNNKYYFNPD TAIISKGWQT VNGSRYYFDT |
| | DTAIAFNGYK TIDGKHFYFD SDCVVKIGVF STSNGFEYFA PANTYNNNIE |
| | GQAIVYQSKF LTLNGKKYYF DNNSKAVTGW QTIDSKKYYF NTNTAEAATG |
| | WQTIDGKKYY FNTNTAEAAT GWQTIDGKKY YFNTNTAIAS TGYTIINGKH |
| | FYFNTDGIMQ IGVFKGPNGF EYFAPANTDA NNIEGQAILY QNEFLTLNGK |
| | KYYFGSDSKA VTGWRIINNK KYYFNPNNAI AAIHLCTINN DKYYFSYDGI |
| | LQNGYITIER NNFYFDANNE SKMVTGVFKG PNGFEYFAPA NTHNNNIEGQ |
| | AIVYQNKFLT LNGKKYYFDN DSKAVTGWQT IDGKKYYFNL NTAEAATGWQ |
| | TIDGKKYYFN LNTAEAATGW QTIDGKKYYF NTNTFIASTG YTSINGKHFY |
| | FNTDGIMQIG VFKGPNGFEY FAPANTDANN IEGQAILYQN KFLTLNGKKY |
| | YFGSDSKAVT GLRTIDGKKY YFNTNTAVAV TGWQTINGKK YYFNTNTSIA |
| | STGYTIISGK HFYFNTDGIM QIGVFKGPDG FEYFAPANTD ANNIEGQAIR |
| | YQNRFLYLHD NIYYFGNNSK AATGWVTIDG NRYYFEPNTA MGANGYKTID |
| | NKNFYFRNGL PQIGVFKGSN GFEYFAPANT DANNIEGQAI RYQNRFLHLL |
| | GKIYYFGNNS KAVTGWQTIN GKVYYFMPDT AMAAAGGLFE IDGVIYFFGV |
| | DGVKAPGIYG |
| 5^{e} | MSLISKEELI KLAYSIRPRE NEYKTILTNL DEYNKLTINN NENKYLQLKK |
| | LNESIDVFMN KYKNSSRNRA LSNLKKDILK EVILIKNSNT SPVEKNLHFV |
| | WIGGEVSDIA LEYIKQWADI NAEYNIKLWY DSEAFLVNTL KKAIVESSTT |
| | EALQLLEEEI QNPQFDNMKF YKKRMEFIYD RQKRFINYYK SQINKPTVPT |
| | IDDIIKSHLV SEYNRDETLL ESYRTNSLRK INSNHGIDIR ANSLFTEQEL |
| | LNIYSQELLN RGNLAAASDI VRLLALKNFG GVYLDVDMLP GIHSDLFKTI |
| | PRPSSIGLDR WEMIKLEAIM KYKKYINNYT SENFDKLDQQ LKDNFKLIIE |
| | SKSEKSEIFS KLENLNVSDL EIKIAFALGS VINQALISKQ GSYLTNLVIE |
| | QVKNRYQFLN QHLNPAIESD NNFTDTTKIF HDSLFNSATA ENSMFLTKIA |
| | PYLQVGFMPE ARSTISLSGP GAYASAYYDF INLQENTIEK TLKASDLIEF |
| | KFPENNLSQL TEQEINSLWS FDQASAKYQF EKYVRDYTGG SLSEDNGVDF |
| | NKNTALDKNY LLNNKIPSNN VEEAGSKNYV HYIIQLQGDD ISYEATCNLF |
| | SKNPKNSIII QRNMNESAKS YFLSDDGESI LELNKYRIPE RLKNKEKVKV |
| | TFIGHGKDEF NTSEFARLSV DSLSNEISSF LDTIKLDISP KNVEVNLLGC |
| | NMFSYDFNVE ETYPGKLLLS IMDKITSTLP DVNKDSITIG ANQYEVRINS |
| | EGRKELLAHS GKWINKEEAI MSDLSSKEYI FFDSIDNKLK AKSKNIPGLA |
| | SISEDIKTLL LDASVSPDTK FILNNLKLNI ESSIGDYIYY EKLEPVKNII |
| | HNSIDDLIDE FNLLENVSDE LYELKKLNNL DEKYLISFED ISKNNSTYSV |
| | RFINKSNGES VYVETEKEIF SKYSEHITKE ISTIKNSIIT DVNGNLLDNI |
| | QLDHTSQVNT LNAAFFIQSL IDYSSNKDVL NDLSTSVKVQ LYAQLFSTGL |
| | NTIYDSIQLV NLISNAVNDT INVLPTITEG IPIVSTILDG INLGAAIKEL |
| | LDEHDPLLKK ELEAKVGVLA INMSLSIAAT VASIVGIGAE VTIFLLPIAG |
| | ISAGIPSLVN NELILHDKAT SVVNYFNHLS ESKEYGPLKT EDDKILVPID |
| | DLVISEIDFN NNSIKLGTCN ILAMEGGSGH TVTGNIDHFF SSPYISSHIP |
| | SLSVYSAIGI KTENLDFSKK IMMLPNAPSR VFWWETGAVP GLRSLENNGT |
| | KLLDSIRDLY PGKFYWRFYA FFDYAITTLK PVYEDTNTKI KLDKDTRNFI |
| | MPTITTDEIR NKLSYSFDGA GGTYSLLLSS YPISMNINLS KDDLWIFNID |
| | NEVREISIEN GTIKKGNLIE DVLSKIDINK NKLIIGNOTI DFSGDIDNKD |
| | RYIFLTCELD DKISLIIEIN LVAKSYSLLL SGDKNYLISN LSNTIEKINT |
| | LGLDSKNIAY NYTDESNNKY FGAISKTSQK SIIHYKKDSK NILEFYNGST |
| | LEFNSKDFIA EDINVFMKDD INTITGKYYV DNNTDKSIDF SISLVSKNQV |
| | KVNGLYLNES VYSSYLDFVK NSDGHHNTSN FMNLFLNNIS FWKLFGFENI |
| | NFVIDKYFTL VGKTNLGYVE FICDNNKNID IYFGEWKTSS SKSTIFSGNG |
| | RNVVVEPIYN PDTGEDISTS LDFSYEPLYG IDRYINKVLI APDLYTSLIN |
| | INTNYYSNEY YPEIIVLNPN TFHKKVNINL DSSSFEYKWS TEGSDFILVR |
| | YLEESNKKIL QKIRIKGILS NTQSFNKMSI DFKDIKKLSL GYIMSNFKSF |
| | NSENELDRDH LGFKIIDNKT YYYDEDSKLV KGLININNSL FYFDPIESNL |
| | VTGWQTINGK KYYFDINTGA ASTSYKIING KHFYFNNNGV MQLGVFKGPD |
| | GFEYFAPANT QNNNIEGOAI VYQSKELTLN GKKYYFDNDS KAVTGWRIIN |
| | NEKYYFNPNN AIAAVGLQVI DNNKYYFNPD TAIISKGWQT VNGSRYYFDT |
| | DTAIAFNGYK TIDGKHFYFD SDCVVKIGVF SGSNGFEYFA PANTYNNNIE |
| | GQAIVYQSKF LTLNGKKYYF DNNSKAVTGW QTIDSKKYYF NTNTAEAATG |
| | WQTIDGKKYY FNTNTAEAAT GWQTIDGKKY YFNTNTSIAS TGYTIINGKY |
| | FYFNTDGIMQ IGVFKVPNGF EYFAPANTHN NNIEGOAILY QNKFLTLNGK |
| | KYYFGSDSKA ITGWQTIDGK KYYFNPNNAI AATHLCTINN DKYYFSYDGI |
| | LQNGYITIER NNFYFDANNE SKMVTGVFKG PNGFEYFAPA NTHNNNIEGQ |
| | AIVYQNKFLT LNGKKYYFDN DSKAVTGWQT IDSKKYYFNL NTAVAVTGWQ |
| | TIDGEKYYFN LNTAEAATGW QTIDGKRYYF NTNTYIASTG YTIINGKHFY |
| | FNTDGIMQIG VFKGPDGFEY FAPANTHNNN IEGQAILYQN KFLTLNGKKY |
| | YFGSDSKAVT GLRTIDGKKY YFNTNTAVAV TGWQTINGKK YYFNTNTYIA |
| | STGYTIISGK HFYFNTDGIM QIGVFKGPDG FEYFAPANTD ANNIEGQAIR |
| | YQNRFLYLHD NIYYFGNDSK AATGWATIDG NRYYFEPNTA MGANGYKTID |
| | NKNFYFRNGL PQIGVFKGPN GFEYFAPANT DANNIDGQAI RYQNRFLHLL |
| | GKIYYFGNNS KAVTGWQTIN SKVYYFMPDT AMAAAGGLFE IDGVIYFFGV |
| | DGVKAPGIYG |
| 6^{f} | GLININNSLF YFDPIESNLV TGWQTINGKK YYFDINTGAA STSYKIINGK |
| | HFYENNNGVM QLGVFKGPDG FEYFAPANTQ NNNIEGOAIV YQSKELTLNG |
| | KKYYFDNDSK AVTGWRIINN EKYYENPNNA IAAVGLQVID NNKYYENPDT |
| | AIISKGWQTV NGSRYYFDTD TAIAFNGYKT IDGKHFYFDS DCVVKIGVFS |
| | GSNGFEYFAP ANTYNNNIEG QAIVYQSKFL TLNGKKYYED NNSKAVTGWQ |
| | TIDSKKYYFN TNTAEAATGW QTIDGKKYYF NTNTAEAATG WQTIDGKKYY |
| | FNTNTSIAST GYTIINGKYF YFNTDGIMQI GVFKVPNGFE YFAPANTHNN |
| | NIEGQAILYQ NKELTLNGKK YYFGSDSKAI TGWQTIDGKK YYFNPNNAIA |
| | ATHLCTINND KYYFSYDGIL QNGYITIERN NFYFDANNES KMVTGVFKGP |
| | NGFEYFAPAN THNNNIEGQA IVYQNKFLTL NGKKYYFDND SKAVTGWQTI |
| | DSKKYYFNLN TAVAVTGWQT IDGEKYYFNL NTAEAATGWQ TIDGKRYYFN |
| | TNTYIASTGY TIINGKHFYF NTDGIMQIGV FKGPDGFEYF APANTHNNNI |
| | EGQAILYQNK FLTLNGKKYY FGSDSKAVTG LRTIDGKKYY FNTNTAVAVT |
| | GWQTINGKKY YFNINTYIAS TGYTIISGKH FYFNTDGIMQ IGVFKGPDGF |
| | EYFAPANTDA NNIEGQAIRY QNRFLYLHDN IYYFGNDSKA ATGWATIDGN |
| | RYYFEPNTAM GANGYKTIDN KNFYFRNGLP QIGVFKGPNG FEYFAPANTD |
| | ANNIDGQAIR YQNRFLHLLG KIYYFGNNSK AVTGWQTINS KVYYFMPDTA |
| | MAAAGGLFEI DGVIYFFGVD GVKAPGIYG |
| 7ª | SPNIYTDEIN ITPVYETN |
| 8ª | YPEVIVLDAN YINEKI |
| 9ª | TVGDDKYYFN PINGG |
| 10ª | ASIGETIIDD KNYYFNQS |
| 11ª | EDGFKYFAPA NTLDEN |
| 12ª | PANTLDENLE GE |
| 13ª | AIDFTGKLII DE |
| 14ª | NIYYFDDNYR GAVE |
| 15ª | HYFSPETGKA FK |
| 16ª | IGDYKYFNSD GVM |
| 17ª | HFYFAENGEM QIGVENTEDG FK |
| 18ª | INDGQYYFND DGIMQV |
| 19ª | YKYFAPANTV NDNIYG |
| 20ª | ESDKYYFNPE TKKA |
| 21ª | NNNYYFNENG EMQFGYINI |
| 22ª | QNTLDENFEG ESINYT |
| 23^{b} | SPNIYTDEIN ITPIYEAN |
| 24^{b} | YPEVIVLDTN YISEKI |
| 25^{b} | TIGDDKYYFN PDNGG |
| 26^{b} | ASVGETIIDG KNYYFSQN |
| 27^{b} | EDGFKYFAPA DTLDEN |
| 28^{b} | PADTLDENLE GE |
| 29^{b} | AIDFTGKLTI DE |
| 30^{b} | NVYYFGDNYR AAIE |
| 31^{b} | YYFSTDTGRA FK |
| 32^{b} | IGDDKFYFNS DGIM |
| 33^{b} | YFYFAENGEM QIGVFNTADG FK |
| 34^{b} | INDGKYYFND SGIMQI |
| 35^{b} | YKYFAPANTV NDNIY |
| 36^{b} | ESDKYYFDPE TKKA |
| 37^{b} | DNHYYFNEDG IMQYGYLNI |
| 38^{b} | QNTLDENFEG ESINYT |
| 39^{g} | DDNGIVQIGV FDTSDGYKYF APANTVNDNI YGQAVEYSGL VRVGEDVYYF |
| | GETYTIETGW IYDMENESDK YYFNPETKKA CKGINLIDDI KYYFDEKGIM |
| | RTGLISFENN NYYFNENGEM QFGYINIEDK MFYFGEDGVM QIGVENTPDG |
| | FKYFAHQNTL DENFEGESIN YTGWLDLDEK RYYFTDEYIA |
| 40^{h} | DENGLVQIGV FDTSDGYKYF APANTVNDNI YGQAVEYSGL VRVGEDVYYF |
| | GETYTIETGW IYDMENESDK YYFDPETKKA YKGINVIDDI KYYFDENGIM |
| | RTGLITFEDN HYYFNEDGIM QYGYLNIEDK TFYFSEDGIM QIGVENTPDG |
| | FKYFAHQNTL DENFEGESIN YTGWLDLDEK RYYFTDEYIA |

^{a}C. difficile Toxin B of VPI 10463
^{b}C. difficile Toxin B of NAP1/027 strain
^{c}C. difficile TcdB2₁₆₅₁₋₂₃₆₆
^{d}C. difficile Toxin A of VPI 10463
^{e}C. difficile Toxin A of NAP1/027 strain
^{f}CROPs region of Toxin A from C. difficile NAP1/027 strain
^{g}C. difficile TcdB₂₁₅₂₋₂₃₄₁ of VPI 10463
^{h}C. difficile TcdB₂₁₅₂₋₂₃₄₁ of BI/NAP1/027 strain

In some embodiments, a vaccine composition of the disclosure can comprise a mixture of two or more C. difficile polypeptides. In some embodiments, a vaccine composition of the disclosure can comprise 2, 3, 4, 5, 6, 7, 8, 9, or 10 C. difficile polypeptides. In some embodiments, a vaccine composition of the disclosure can comprise 2 C. difficile polypeptides. In some embodiments, a vaccine composition of the disclosure can comprise 3 C. difficile polypeptides. In some embodiments, a vaccine composition of the disclosure can comprise 4 C. difficile polypeptides. In some embodiments, a vaccine composition of the disclosure can comprise at least one of a C. difficile Toxin A toxoid; a C. difficile Toxin B toxoid; a non-toxic fragment of a C. difficile Toxin A; a non-toxic fragment of C. difficile Toxin B; or any combination thereof.

In some embodiments, a polypeptide or polypeptide fragment of C. difficile Toxin A or Toxin B can comprise at least one mutation. In some embodiments, a polypeptide or polypeptide fragment of C. difficile Toxin A or Toxin B comprises 1, 2, 3, 4, 5, 6, 7, 8, 9, 10, 11, 12, 13, 14, 15, 16, 17, 18, 19, or 20 mutations. In some embodiments, the mutation is a deletion. In some embodiments, the mutation is a substitution. In some embodiments, the mutation is an insertion. In some embodiments, the mutation is a truncation.

In some embodiments, the polypeptide or polypeptide fragment of C. difficile Toxin A or Toxin B comprises at least one mutation in the glucosyl-transferase domain. In some embodiments, the mutation in the glucosyl-transferase domain is at amino acids 1-541 of SEQ ID NO: 4. In some embodiments, the mutation in the glucosyl-transferase domain is at amino acids 1-541 of SEQ ID NO: 5. In some embodiments, the mutation in the glucosyltransferase domain is at amino acids 1-543 of SEQ ID NO: 1. In some embodiments, the mutation in the glucosyltransferase domain is at amino acids 1-543 of SEQ ID NO: 2.

In some embodiments, a polypeptide or polypeptide fragment of C. difficile Toxin B can comprise 1, 2, 3, 4, 5, 6, 7, 8, 9, 10, 11, 12, 13, 14, 15, 16, 17, 18, 19, or 20 substitutions relative to wild type Toxin B antigen sequence of SEQ ID NO: 1 or SEQ ID NO: 2. In some embodiments, a polypeptide or polypeptide fragment of C. difficile Toxin B can comprise 1, 2, 3, 4, or 5 positions corresponding to amino acids 270, 273, 284, 286, or 288 of SEQ ID NO: 1 or SEQ ID NO: 2. In some embodiments, a polypeptide or polypeptide fragment of C. difficile Toxin A can comprise 1, 2, 3, 4, 5, 6, 7, 8, 9, 10, 11, 12, 13, 14, 15, 16, 17, 18, 19, or 20 substitutions relative to wild type Toxin A antigen sequence of SEQ ID NO: 4 or SEQ ID NO: 5. In some embodiments, a polypeptide or polypeptide fragment of C. difficile Toxin A can comprise substitutions at 1, 2, or 3 positions corresponding to amino acids 283, 285, or 287 of SEQ ID NO: 4 or SEQ ID NO: 5.

In some embodiments, a polypeptide or polypeptide fragment of C. difficile Toxin A or Toxin B comprises at least one mutation in the cutting or cysteine protease enzymatic domain. In some embodiments, the cutting or cysteine protease enzymatic domain is at amino acids 542-769 of SEQ ID NO: 4; amino acids 542-769 of SEQ ID NO: 5; amino acids 544-767 of SEQ ID NO: 1; or amino acids 544-767 of SEQ ID NO: 2. In some embodiments, a polypeptide or polypeptide fragment of C. difficile Toxin A or Toxin B comprises at least one mutation in the translocation domain. In some embodiments, the translocation domain is at amino acids 770-1808 of SEQ ID NO: 4; amino acids 770-1808 of SEQ ID NO: 5; amino acids 768-1833 of SEQ ID NO: 1; or amino acids 768-1833 of SEQ ID NO: 2.

In some embodiments, a polypeptide or polypeptide fragment of C. difficile Toxin A or Toxin B has a minimum length of about 10, 20, 30, 40, 50, 60, 70, 80, 90, 100, 125, 150, 175, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, or 2650 amino acids. In some embodiments, the polypeptide or polypeptide fragment has a minimum length of about 10, 20, 30, 40, 50, 60, 70, 80, 90, 100, 125, 150, 175, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, or 2650 amino acids from TcdA₁₆₄₉₋₂₇₁₀, TcdB₁₆₅₁₋₂₃₆₆, the CROP region of Toxin A (TcdA₁₈₃₂₋₂₇₁₀), or the CROP region of Toxin B (TcdB₁₈₃₄₋₂₃₆₆).

In some embodiments, a vaccine composition of the disclosure can comprise two or more Toxin A, Toxin B, or fragments thereof. In some embodiments, the two or more Toxin A or Toxin B fragments are identical. In some embodiments, the two or more Toxin A or Toxin B fragments are different. In some embodiments, the two or more Toxin A or Toxin B fragments are contiguous. In some embodiments, the two or more Toxin A or Toxin B fragments are non-contiguous. In some embodiments, the two or more Toxin A or Toxin B fragments are linked in any order, for example, A-A-B, A-B-A, B-A-A, B-B-A, B-A-B, A-B-A, or A-B-B from N-terminus to C-terminus.

In some embodiments, a vaccine composition of the disclosure can comprise TcdA and TcdB in a TcdA:TcdB ratio of about 90:10, about 80:20, about 70:30, about 60:40, about 50:50, about 40:60, about 30:70, about 20:80, or about 10:90. In some embodiments, a vaccine composition of the disclosure can comprise TcdA and TcdB in a TcdA:TcdB ratio of about 50:50 (i.e., an equal amount). In some embodiments, a vaccine composition of the disclosure can comprise TcdA and TcdB in a TcdA:TcdB ratio of about 70:30. In some embodiments, a vaccine composition of the disclosure can comprise TcdA and TcdB in a TcdA:TcdB ratio of about 30:70. In some embodiments, a vaccine composition of the disclosure can comprise TcdA and TcdB in a TcdA:TcdB ratio of about 80:20. In some embodiments, a vaccine composition of the disclosure can comprise TcdA and TcdB in a TcdA:TcdB ratio of about 20:80.

### Pharmaceutical Compositions

In some embodiments, disclosed herein is a vaccine composition targeting C. difficile bacteria and at least one C. difficile enterotoxin. In some embodiments, a vaccine composition of the disclosure targets C. difficile bacteria. In some embodiments, a vaccine composition of the disclosure targets C. difficile bacteria and one C. difficile enterotoxin, for example, Toxin A or Toxin B. In some embodiments, a vaccine composition of the disclosure targets C. difficile bacteria and two C. difficile enterotoxins, for example, Toxin A and Toxin B.

A pharmaceutical composition of the invention can be a combination of any compounds or pharmaceutically acceptable salts described herein with other chemical components, such as carriers, stabilizers, diluents, dispersing agents, suspending agents, thickening agents, adjuvants, and/or excipients. The pharmaceutical compositions of the disclosure can facilitate administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. Subjects can be, for example, elderly adults, adults, adolescents, pre-adolescents, children, toddlers, infants, neonates, and non-human animals. In some embodiments, a subject is a patient. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions of the disclosure can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A pharmaceutical composition of the disclosure can comprise an adjuvant. In some embodiments, the adjuvant can accelerate, prolong, or enhance an antigen-specific immune response when used in combination with an antigen or compound of the disclosure. In some embodiments, the adjuvant can stimulate the immune system to respond to the vaccine more vigorously and provide increased immunity against an infection. In some embodiments, the adjuvant is an inorganic compound, and oil, a bacterial product, a plant product, a cytokine, an organic substance, or a combination of agents.

In some embodiments, the adjuvant is an inorganic compound. In some embodiments, the inorganic compound is potassium alum, aluminum potassium sulfate, aluminum hydroxide, aluminum phosphate, or calcium phosphate hydroxide. In some embodiments, the adjuvant is an oil. In some embodiments, the oil is paraffin oil, adjuvant 65, or peanut oil. In some embodiments, the adjuvant is a bacterial product, for example, killed bacteria of the species Bordetella pertussis, Mycobacterium bovis, or a toxoid. In some embodiments, the adjuvant is a plant product, for example, a plant saponin from Quillaja, soybean, or polysaga senega. In some embodiments, the adjuvant is a cytokine, for example, IL-1, IL-2, or IL-12. In some embodiments, the adjuvant is a combination adjuvant. In some embodiments, the combination adjuvant is Freund's complete adjuvant or Freund's incomplete adjuvant. In some embodiments, the adjuvant is an organic substance, for example, squalene.

In some embodiments, the adjuvant is alum, mineral oil, a vegetable oil, aluminum hydroxide, Freund's incomplete adjuvant, or a TLR agonist (e.g., CpG oligonucleotide). In some embodiments, the adjuvant is AS04 (i.e., monophosphoryl lipid A and aluminum salt). In some embodiments, the adjuvant is MF59 (i.e., oil in water emulsion with squalene). In some embodiments, the adjuvant is AS01B (i.e., monophosphoryl lipid A and QS-21 combined in a liposomal formulation). In some embodiments, the adjuvant is CpG 1018 (i.e., cytosine phosphoguanine). In some embodiments, the adjuvant is AlPO₄. In some embodiments, the adjuvant is AlOH. In some embodiments, the adjuvant is Advax™ or Advax-2™ (delta inulin microparticles).

In some embodiments, the pharmaceutical composition comprises one adjuvant. In some embodiments, the pharmaceutical composition comprises two adjuvants. In some embodiments, the pharmaceutical composition comprises three adjuvants. In some embodiments, the pharmaceutical composition comprises four adjuvants. In some embodiments, the pharmaceutical composition comprises five adjuvants. In some embodiments, the pharmaceutical composition does not comprise an adjuvant.

In some embodiments, disclosed herein is a composition comprising a cell-surface polysaccharide from Clostridioides difficile (C. difficile), wherein the cell-surface polysaccharide is a PSII antigen enriched from C. difficile, wherein the composition has a total carbohydrate percentage by weight of at least about 40%, and wherein the PSII antigen enriched from C. difficile is at least 90% of the total carbohydrate percentage.

In some embodiments, disclosed herein is a composition comprising: (a) a cell-surface polysaccharide of Clostridioides difficile (C. difficile); and (b) a first polypeptide comprising a carrier protein; wherein the carrier protein and the cell-surface polysaccharide of C. difficile are present in the composition at a ratio of less than about 10:1 to about 1:3. Disclosed herein is a composition comprising: (a) a cell-surface polysaccharide of Clostridioides difficile (C. difficile); and (b) a first polypeptide comprising a carrier protein derived from an organism other than C. difficile; wherein the carrier protein and the cell-surface polysaccharide are present in the composition at a ratio of from about 10:1 to about 1:10.

In some embodiments, disclosed herein is a pharmaceutical composition comprising: (a) a cell-surface polysaccharide enriched from Clostridioides difficile (C. difficile); (b) a first polypeptide or a first polynucleotide encoding the first polypeptide, wherein the first polypeptide comprises a first toxoid of C. difficile or a fragment thereof; (c) a second polypeptide or a second polynucleotide encoding the second polypeptide, wherein the second polypeptide comprises a second toxoid of C. difficile or a fragment thereof; and (d) a pharmaceutically acceptable carrier; wherein the pharmaceutical composition has a total carbohydrate percentage, wherein at least about 90% of the total carbohydrate percentage is PSII.

In some embodiments, disclosed herein is a pharmaceutical composition comprising: (a) a cell-surface polysaccharide enriched from Clostridioides difficile (C. difficile); (b) a first polypeptide comprising a first toxoid of C. difficile or a fragment thereof; (c) a second polypeptide comprising a second toxoid of C. difficile or a fragment thereof; and (d) a pharmaceutically acceptable carrier; wherein the cell-surface polysaccharide enriched from C. difficile is not conjugated to the first polypeptide or the second polypeptide.

In some embodiments, disclosed herein is a pharmaceutical composition comprising: (a) a cell-surface polysaccharide of Clostridioides difficile (C. difficile); and (b) a first polypeptide or a polynucleotide encoding the first polypeptide, wherein the polypeptide comprises a first toxoid of C. difficile or a fragment thereof that is an inactivated toxin; wherein the pharmaceutical composition comprises less than about 5% by weight of a C. difficile protein other than an inactivated toxin of C. difficile.

In some embodiments, disclosed herein is a pharmaceutical composition comprising: (a) a cell-surface polysaccharide from Clostridioides difficile (C. difficile); (b) a first polypeptide or a first polynucleotide encoding the first polypeptide, wherein the first polypeptide comprises a first toxoid of C. difficile or a fragment thereof; (c) a second polypeptide or a second polynucleotide encoding the second polypeptide, wherein the second polypeptide comprises a second toxoid of C. difficile or a fragment thereof, and (d) a pharmaceutically acceptable carrier; wherein the first toxoid is a full-length toxin A toxoid and the second toxoid is a full-length toxin B toxoid.

In some embodiments, disclosed herein is a pharmaceutical composition comprising: (a) a cell-surface polysaccharide of Clostridioides difficile (C. difficile); and (b) a first polypeptide or a polynucleotide encoding the first polypeptide, wherein the first polypeptide comprises a first inactivated toxin of C. difficile or a fragment thereof, wherein the pharmaceutical composition comprises less than about 5% by weight of a C. difficile protein other than an inactivated toxin of C. difficile. Also disclosed herein is a pharmaceutical composition comprising a cell-surface polysaccharide of Clostridioides difficile (C. difficile) conjugated to a carrier protein; wherein the cell-surface polysaccharide of C. difficile is conjugated to the carrier protein via a chemical linker comprising from 1 carbon to 10 carbon atoms.

In some embodiments, disclosed herein is a pharmaceutical composition comprising: (a) a cell-surface polysaccharide from Clostridioides difficile (C. difficile) conjugated to a carrier protein; (b) a first polypeptide comprising a first toxoid of C. difficile or a fragment thereof; (c) a second polypeptide comprising a second toxoid of C. difficile or a fragment thereof; and (d) a pharmaceutically acceptable carrier; wherein the cell-surface polysaccharide from C. difficile is conjugated to the carrier protein via a chemical linker selected from the group consisting of a thioether, an adipic acid dihydrazide, a urea, or an amine.

In some embodiments, disclosed herein is a pharmaceutical composition consisting essentially of: (a) a cell-surface polysaccharide from Clostridioides difficile (C. difficile) conjugated to a pharmaceutically acceptable carrier; (b) a first polypeptide or a first polynucleotide encoding the first polypeptide, wherein the first polypeptide comprises a first toxoid of C. difficile or a fragment thereof; (c) a second polypeptide or a second polynucleotide encoding the second polypeptide, wherein the second polypeptide comprises a second toxoid of C. difficile or a fragment thereof; and (d) an adjuvant.

In some embodiments, also disclosed herein are methods of making a immunogenic composition by admixing an antigen or cell-surface polysaccharide of C. difficile or a cell-surface polysaccharide conjugate with at least one of toxin A, toxin B, or a fragment thereof. In some embodiments, toxin A, toxin B, or a fragment thereof is a non-toxic, immunogenic polypeptide. In some embodiments, the immunogenic composition can elicit a protective immune response by facilitating the production of antibodies reactive with one or more strains of C. difficile and antibodies reactive with one or more C. difficile toxins. In some embodiments, the immunogenic composition can facilitate the production of antibodies reactive with C. difficile Toxin A. In some embodiments, the immunogenic composition can facilitate the production of antibodies reactive with C. difficile Toxin B. In some embodiments, the immunogenic composition can facilitate the production of antibodies reactive with C. difficile Toxin A and Toxin B.

In some embodiments, the non-toxic polypeptide or a fragment thereof is a recombinantly produced polypeptide.

A vaccine composition of the disclosure can have a cell-surface polysaccharide:polypeptide ratio of about 100:1, about 80:1, about 60:1, about 40:1, about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:20, about 1:40, about 1:60, about 1:80, or about 1:100. In some embodiments, a pharmaceutical composition can have a cell-surface polysaccharide:polypeptide ratio of about 10:1. In some embodiments, a pharmaceutical composition can have a cell-surface polysaccharide:polypeptide ratio of about 5:1. In some embodiments, a pharmaceutical composition can have a cell-surface polysaccharide:polypeptide ratio of about 1:1. In some embodiments, the polypeptide is toxoid A or a fragment thereof. In some embodiments, the polypeptide is toxoid B or a fragment thereof.

A vaccine composition of the disclosure can comprise less than about 50%, about 40%, about 30%, about 20%, about 10%, about 9%, about 8%, about 7%, about 6%, about 5%, about 4%, about 3%, about 2%, or about 1% by weight of a polypeptide from C. difficile. In some embodiments, a pharmaceutical composition of the disclosure can comprise less than about 20%, about 15%, about 10%, about 9%, about 8%, about 7%, about 6%, about 5%, about 4%, about 3%, about 2%, or about 1% by weight of a polypeptide from C. difficile. In some embodiments, a pharmaceutical composition of the disclosure can comprise less than about 20% by weight of a polypeptide from C. difficile. In some embodiments, a pharmaceutical composition of the disclosure can comprise less than about 10% by weight of a polypeptide from C. difficile. In some embodiments, a pharmaceutical composition of the disclosure can comprise less than about 5% by weight of a polypeptide from C. difficile.

A pharmaceutical composition of the disclosure can have a total carbohydrate percentage by weight of at least about 30%, about 40%, about 50%, about 60%, about 70%, about 80%, or about 90%. In some embodiments, a pharmaceutical composition of the disclosure can have a total carbohydrate percentage by weight of at least about 40%. In some embodiments, a pharmaceutical composition of the disclosure can have a total carbohydrate percentage by weight of at least about 60%. In some embodiments, a pharmaceutical composition of the disclosure can have a total carbohydrate percentage by weight of at least about 80%.

In some embodiments, at least about 60%, about 65%, about 70%, about 75%, about 80%, about 85%, about 90%, or about 95% of the saccharides present in a pharmaceutical composition of the disclosure is a cell-surface polysaccharide of the disclosure, for example, the PSII antigen. In some embodiments, at least about 80% of the saccharides present in the pharmaceutical composition is the PRI antigen. In some embodiments, at least about 90% of the saccharides present in the pharmaceutical composition is the PRI antigen. In some embodiments, at least about 95% of the saccharides present in the pharmaceutical composition is the PSII antigen.

A vaccine composition of the disclosure can comprise at least about 5%, about 10%, about 15%, about 20%, about 25%, about 30%, about 35%, about 40%, about 45%, or about 50% by weight of a cell-surface polysaccharide from C. difficile. In some embodiments, a composition of the disclosure can comprise at least about 5% by weight of a cell-surface polysaccharide from C. difficile. In some embodiments, a composition of the disclosure can comprise at least about 15% by weight of a cell-surface polysaccharide from C. difficile. In some embodiments, a composition of the disclosure can comprise at least about 25% by weight of a cell-surface polysaccharide from C. difficile.

A vaccine composition of the disclosure can comprise at least about 75% of a cell-surface polysaccharide from C. difficile or a conjugate thereof; at least about 5% of a first toxoid polypeptide of C. difficile or a fragment thereof and at least about 5% of a second toxoid polypeptide of C. difficile or a fragment thereof. A vaccine composition of the disclosure can comprise about 82% of a cell-surface polysaccharide from C. difficile or a conjugate thereof at least about 9% of a first toxoid polypeptide of C. difficile or a fragment thereof and at least about 9% of a second toxoid polypeptide of C. difficile or a fragment thereof. A vaccine composition of the disclosure can comprise about 82% of a PSII-CRM₁₉₇ conjugate of the disclosure; at least about 9% of TcdA of C. difficile or a fragment thereof and at least about 9% of TcdB of C. difficile or a fragment thereof.

A vaccine composition of the disclosure can comprise less than about 5%, about 4.5%, about 4%, about 3.5%, about 3%, about 2.5%, about 2%, about 1.5%, or about 1% of a contaminant. In some embodiments, the contaminant is a peptidoglycan, a protein, a nucleic acid, a saccharide, or a combination thereof. In some embodiments, the contaminant is derived from a cell-surface extract of C. difficile.

A vaccine composition of the disclosure can comprise less than about 5%, about 4.5%, about 4%, about 3.5%, about 3%, about 2.5%, about 2%, about 1.5%, or about 1% of peptidoglycan contamination. In some embodiments, a vaccine composition of the disclosure can comprise less than about 1%, about 0.9%, about 0.8%, about 0.7%, about 0.6%, about 0.5%, about 0.4%, about 0.3%, about 0.2%, or about 0.1% of peptidoglycan contamination. In some embodiments, a vaccine composition of the disclosure can comprise less than about 5% of peptidoglycan contamination. In some embodiments, a vaccine composition of the disclosure can comprise less than about 1% of peptidoglycan contamination. In some embodiments, a vaccine composition of the disclosure can comprise less than about 0.5% of peptidoglycan contamination. In some embodiments, a vaccine composition of the disclosure can comprise less than about 0.3% of peptidoglycan contamination. In some embodiments, the peptidoglycan contamination is relative to the total weight of cell-surface polysaccharide in the composition.

A vaccine composition of the disclosure can comprise less than about 5%, about 4.5%, about 4%, about 3.5%, about 3%, about 2.5%, about 2%, about 1.5%, or about 1% of protein contamination. In some embodiments, a vaccine composition of the disclosure can comprise less than about 1%, about 0.9%, about 0.8%, about 0.7%, about 0.6%, about 0.5%, about 0.4%, about 0.3%, about 0.2%, or about 0.1% of protein contamination. In some embodiments, a vaccine composition of the disclosure can comprise less than about 5% of protein contamination. In some embodiments, a vaccine composition of the disclosure can comprise less than about 1% of protein contamination. In some embodiments, a composition of the disclosure can comprise less than about 0.5% of protein contamination. In some embodiments, a composition of the disclosure can comprise less than about 0.3% of protein contamination. In some embodiments, the protein contamination is a C. difficile protein other than an inactivated toxin of C. difficile. In some embodiments, the protein contamination is relative to the total weight of cell-surface polysaccharide in the composition.

A vaccine composition of the disclosure can comprise less than about 5%, about 4.5%, about 4%, about 3.5%, about 3%, about 2.5%, about 2%, about 1.5%, or about 1% nucleic acid contamination. In some embodiments, a vaccine composition of the disclosure can comprise less than about 1%, about 0.9%, about 0.8%, about 0.7%, about 0.6%, about 0.5%, about 0.4%, about 0.3%, about 0.2%, or about 0.1% of nucleic acid contamination. In some embodiments, a vaccine composition of the disclosure can comprise less than about 1% of nucleic acid contamination. In some embodiments, a vaccine composition of the disclosure can comprise less than about 0.5% of nucleic acid contamination. In some embodiments, a vaccine composition of the disclosure can comprise less than about 0.3% of nucleic acid contamination. In some embodiments, the nucleic acid contamination is relative to the total weight of cell-surface polysaccharide in the composition.

A vaccine composition of the disclosure can comprise less than about 5%, about 4.5%, about 4%, about 3.5%, about 3%, about 2.5%, about 2%, about 1.5%, or about 1% a saccharide contamination. In some embodiments, a vaccine composition of the disclosure can comprise less than about 1%, about 0.9%, about 0.8%, about 0.7%, about 0.6%, about 0.5%, about 0.4%, about 0.3%, about 0.2%, or about 0.1% of saccharide contamination. In some embodiments, a vaccine composition of the disclosure can comprise less than about 1% of saccharide contamination. In some embodiments, a vaccine composition of the disclosure can comprise less than about 0.5% of saccharide contamination. In some embodiments, a vaccine composition of the disclosure can comprise less than about 0.3% of saccharide contamination. In some embodiments, the saccharide contamination is relative to the total weight of cell-surface polysaccharide in the composition.

A vaccine composition of the disclosure can comprise an endotoxin level that is less than about 50 EU/mg, about 40 EU/mg, about 30 EU/mg, about 20 EU/mg, or about 10 EU/mg of purified PSII. In some embodiments, a vaccine composition of the disclosure can comprise an endotoxin level of less than about 10 EU/mg, about 9 EU/mg, about 8 EU/mg, about 7 EU/mg, about 6 EU/mg, about 5 EU/mg, about 4 EU/mg, about 3 EU/mg, about 2 EU/mg, or about 1 EU/mg of purified PRI. In some embodiments, a vaccine composition of the disclosure can comprise an endotoxin level of less than 10 EU/mg of purified PRI. In some embodiments, a composition of the disclosure can comprise an endotoxin level of less than 5 EU/mg of purified PRI.

### Method of Administration and Dosing

The components of the vaccine composition can be administered together or separately. In some embodiments, a vaccine composition comprising a cell-surface polysaccharide or cell-surface polysaccharide conjugate and at least one C. difficile toxin is prepared in unit dosage form. In some embodiments, the cell-surface polysaccharide or cell-surface polysaccharide conjugate and at least one C. difficile toxin are administered by the same route of administration. In some embodiments, the cell-surface polysaccharide or cell-surface polysaccharide conjugate and the at least one C. difficile toxin are administered separately. In some embodiments, the cell-surface polysaccharide or cell-surface polysaccharide conjugate and at least one C. difficile toxin are administered by different routes of administration. In some embodiments, the cell-surface polysaccharide or cell-surface polysaccharide conjugate and at least one C. difficile toxin are administered concomitantly. In some embodiments, the cell-surface polysaccharide or cell-surface polysaccharide conjugate and at least one C. difficile toxin are administered sequentially.

In some embodiments, a vaccine composition of the disclosure is administered using parenteral routes (e.g., intraperitoneal (i.p.), intramuscular (i.m.), or subcutaneous (s.c.) injection), oral administration, or mucosal (e.g., intranasal (i.n.), intragastric (i.g.), intravaginal, rectal) administration. In some embodiments, a vaccine composition of the disclosure is administered parenterally. In some embodiments, a vaccine composition of the disclosure is administered subcutaneously. In some embodiments, a vaccine composition of the disclosure is administered intramuscularly.

A vaccine composition of the disclosure can be administered on a single dose schedule or a multiple dose schedule. In some embodiments, multiple doses of the components or vaccine composition can be administered on a primary immunization schedule and a booster immunization schedule. In some embodiments, the multiple doses of the components can be administered together. In some embodiments, the multiple doses of the components can be administered separately, for example, at different times or using different routes of administration. In some embodiments, the multiple doses of the components can be administered using different routes of administration, for example, a parenteral prime and mucosal boost, or a mucosal prime and parenteral boost.

In some embodiments, a primary immunization is administered by intramuscular injection. In some embodiments, a secondary or booster immunization is administered by intramuscular injection. In some embodiments, a secondary or booster immunization is administered by oral administration.

In some embodiments, the multiple doses are administered at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, or about 16 weeks apart. In some embodiments, a subject can be immunized with one dose. In some embodiments, a subject can be immunized with two doses about 2 weeks apart. In some embodiments, a subject can be immunized with two doses about 3 weeks apart. In some embodiments, a subject can be immunized with two doses about 4 weeks apart. In some embodiments, a subject can be immunized with two doses about 6 weeks apart. In some embodiments, a subject can be immunized with three doses, wherein each dose is about 2 weeks apart. In some embodiments, a subject can be immunized with three doses, wherein each dose is about 3 weeks apart. In some embodiments, a subject can be immunized with three doses, wherein each dose is about 4 weeks apart.

In some embodiments, a vaccine composition of the disclosure can be administered to prime a subject's immune system with a first immunization. In some embodiments, a vaccine composition of the disclosure can be administered a second time as a booster shot to the subject's “primed” immune system. In some embodiments, a booster dose can be administered at any time during the infection, for example, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days after CDI symptoms are detected or observed.

In some embodiments, a vaccine composition of the disclosure can be administered to a subject to restore immunity to protective levels, for example, in a subject that was previously immunized by vaccination or who has recovered from CDI following treatment. In some embodiments, a booster immunization can be administered about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, or about 20 years after initial immunization or CDI treatment. In some embodiments, a booster immunization can be administered about 3 years after initial immunization or CDI treatment. In some embodiments, a booster immunization can be administered about 5 years after initial immunization or CDI treatment. In some embodiments, a booster immunization can be administered about 7 years after initial immunization or CDI treatment. In some embodiments, a booster immunization can be administered about 10 years after initial immunization or CDI treatment. In some embodiments, a booster immunization can be administered about 15 years after initial immunization or CDI treatment. In some embodiments, a booster immunization can be administered about 20 years after initial immunization or CDI treatment.

The vaccine compositions of the disclosure can be administered prophylactically to patients at substantially the same time as a second vaccine. In some embodiments, the vaccine compositions of the disclosure can be administered substantially the same time as a pneumonia, measles, mumps, rubella, MMR, varicella, MMRV, diphtheria, tetanus, pertussis, DTP, conjugated H. influenzae type B, inactivated poliovirus, hepatitis B virus, meningococcal conjugate (e.g., tetravalent A-C-W135-Y vaccine), or a respiratory syncytial virus vaccine.

Pharmaceutical compositions, polypeptides, or antigens of the disclosure can be in unit dosage forms suitable for single administration of precise dosages. In unit dosage form, the pharmaceutical compositions, polypeptides, or antigens of the disclosure can be divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or multi-dose containers with a preservative.

In some embodiments, an antigen of the disclosure (e.g., PSI, PSII, or PSIII) can be administered to a subject in an amount ranging from about 5 μg to about 50 μg, from about 50 μg to about 100 μg, from about 100 μg to about 150 μg, from about 150 μg to about 200 μg, from about 200 μg to about 250 μg, from about 250 μg to about 300 μg, from about 300 μg to about 350 μg, from about 350 μg to about 400 μg, from about 400 μg to about 450 μg, from about 450 μg to about 500 μg, from about 500 μg to about 600 μg, or from about 600 μg to about 750 μg. In some embodiments, an antigen of the disclosure (e.g., PSI, PSII, or PSIII) can be administered to a subject in an amount ranging from about 5 μg to about 250 μg. In some embodiments, an antigen of the disclosure (e.g., PSI, PSII, or PSIII) can be administered to a subject in an amount ranging from about 5 μg to about 50 μg. In some embodiments, an antigen of the disclosure (e.g., PSI, PSII, or PSIII) can be administered to a subject in an amount ranging from about 100 μg to about 250 μg. In some embodiments, an antigen of the disclosure (e.g., PSI, PSII, or PSIII) can be administered to a subject in an amount ranging from about 250 μg to about 500 μg.

In some embodiments, an antigen of the disclosure (e.g., PSI, PSII, or PSIII) can be administered to a subject in an amount of about 10 μg, about 20 μg, about 30 μg, about 40 μg, about 50 μg, about 60 μg, about 70 μg, about 80 μg, about 90 μg, about 100 μg, about 120 μg, about 140 μg, about 160 μg, about 180 μg, or about 200 μg. In some embodiments, an antigen of the disclosure (e.g., PSI, PSII, or PSIII) can be administered to a subject in an amount of about 200 μg, about 250 μg, about 300 μg, about 350 μg, about 400 μg, about 450 μg, about 500 μg, about 550 μg, about 600 μg, about 650 μg, about 700 μg, or about 750 μg. In some embodiments, an antigen of the disclosure (e.g., PSI, PSII, or PSIII) can be administered to a subject in an amount of about 10 μg. In some embodiments, an antigen of the disclosure (e.g., PSI, PSII, or PSIII) can be administered to a subject in an amount of about 50 μg. In some embodiments, an antigen of the disclosure (e.g., PSI, PSII, or PSIII) can be administered to a subject in an amount of about 100 μg. In some embodiments, an antigen of the disclosure (e.g., PSI, PSII, or PSIII) can be administered to a subject in an amount of about 250 μg. In some embodiments, an antigen of the disclosure (e.g., PSI, PSII, or PSIII) can be administered to a subject in an amount of about 500 μg.

In some embodiments, a toxoid A of the disclosure or a fragment thereof can be administered to a subject in an amount ranging from about 10 μg to about 50 μg, from about 50 μg to about 100 μg, from about 100 μg to about 150 μg, from about 150 μg to about 200 μg, from about 200 μg to about 250 μg, from about 250 μg to about 300 μg, from about 300 μg to about 350 μg, from about 350 μg to about 400 μg, from about 400 μg to about 450 μg, or from about 450 μg to about 500 μg. In some embodiments, a toxoid A of the disclosure or a fragment thereof can be administered to a subject in an amount ranging from about 10 μg to about 50 μg. In some embodiments, a toxoid A of the disclosure or a fragment thereof can be administered to a subject in an amount ranging from about 50 μg to about 100 μg.

In some embodiments, a toxoid A of the disclosure or a fragment thereof can be administered to a subject in an amount of about 10 μg, about 20 μg, about 30 μg, about 40 μg, about 50 μg, about 60 μg, about 70 μg, about 80 μg, about 90 μg, about 100 μg, about 120 μg, about 140 μg, about 160 μg, about 180 μg, or about 200 μg. In some embodiments, a toxoid A of the disclosure or a fragment thereof can be administered to a subject in an amount of about 30 μg. In some embodiments, a toxoid A of the disclosure or a fragment thereof can be administered to a subject in an amount of about 60 μg. In some embodiments, a toxoid A of the disclosure or a fragment thereof can be administered to a subject in an amount of about 90 μg.

In some embodiments, a toxoid B of the disclosure or a fragment thereof can be administered to a subject in an amount ranging from about 10 μg to about 50 μg, from about 50 μg to about 100 μg, from about 100 μg to about 150 μg, from about 150 μg to about 200 μg, from about 200 μg to about 250 μg, from about 250 μg to about 300 μg, from about 300 μg to about 350 μg, from about 350 μg to about 400 μg, from about 400 μg to about 450 μg, or from about 450 μg to about 500 μg. In some embodiments, a toxoid B of the disclosure or a fragment thereof can be administered to a subject in an amount ranging from about 10 μg to about 50 μg. In some embodiments, a toxoid B of the disclosure or a fragment thereof can be administered to a subject in an amount ranging from about 50 μg to about 100 μg.

In some embodiments, a toxoid B of the disclosure or a fragment thereof can be administered to a subject in an amount of about 10 μg, about 20 μg, about 30 μg, about 40 μg, about 50 μg, about 60 μg, about 70 μg, about 80 μg, about 90 μg, about 100 μg, about 120 μg, about 140 μg, about 160 μg, about 180 μg, or about 200 μg. In some embodiments, a toxoid B of the disclosure or a fragment thereof can be administered to a subject in an amount of about 40 μg. In some embodiments, a toxoid B of the disclosure or a fragment thereof can be administered to a subject in an amount of about 40 μg. In some embodiments, a toxoid B of the disclosure or a fragment thereof can be administered to a subject in an amount of about 60 μg.

In some embodiments, a vaccine composition of the disclosure administered to a subject can comprise at least about 5-200 jig of a cell-surface polysaccharide from C. difficile or a conjugate thereof; at least about 1-100 jig of a first toxoid polypeptide of C. difficile or a fragment thereof; and at least about 1-100 jig of a second toxoid polypeptide of C. difficile or a fragment thereof. In some embodiments, a vaccine composition of the disclosure administered to a subject can comprise at least about 50 jig of a cell-surface polysaccharide from C. difficile or a conjugate thereof; at least about 5 μg of a first toxoid polypeptide of C. difficile or a fragment thereof; and at least about 5 μg of a second toxoid polypeptide of C. difficile or a fragment thereof. In some embodiments, a vaccine composition of the disclosure administered to a subject can comprise at least about 100 jig of a cell-surface polysaccharide from C. difficile or a conjugate thereof; at least about 10 jig of a first toxoid polypeptide of C. difficile or a fragment thereof; and at least about 10 jig of a second toxoid polypeptide of C. difficile or a fragment thereof. In some embodiments, a vaccine composition of the disclosure administered to a subject can comprise at least about 200 jig of a cell-surface polysaccharide from C. difficile or a conjugate thereof; at least about 20 μg of a first toxoid polypeptide of C. difficile or a fragment thereof; and at least about 20 μg of a second toxoid polypeptide of C. difficile or a fragment thereof.

### Methods of Use

In some embodiments, disclosed herein are methods of prophylactically reducing the pathological effects of or preventing CDI in a subject, the method comprising administering to the subject a purified PRI cell-surface polysaccharide or a purified PSII cell-surface polysaccharide conjugate and at least one polypeptide comprising toxin A, toxin B, or a non-toxic, immunogenic polypeptide fragment thereof. The compositions of the disclosure target both bacterial surface antigens presented on C. difficile cells and bacterial toxins produced by the C. difficile cells. The vaccine compositions of the disclosure can be used to prevent CDI or prevent the recurrence of CDI. Serum antibodies against C. difficile surface components are found in patients with symptomatic CDI or asymptomatic carriers of C. difficile.

In some embodiments, the vaccine compositions of the disclosure can be used to immunize a subject that is immunologically naïve to C. difficile, i.e., a person with no former exposure to C. difficile. In some embodiments, the vaccine compositions of the disclosure can be used for therapeutic immunization, for example, to prevent subsequent CDI or recurrent CDI.

In some embodiments, disclosed herein are methods of eliciting an immune response in a subject against C. difficile, the method comprising administering to the subject an amount of an immunogenic composition comprising a purified PSII cell-surface polysaccharide or a purified PSII cell-surface polysaccharide conjugate and at least one of toxin A, toxin B, or a fragment thereof.

In some embodiments, the compositions and methods of the disclosure can elicit a cell-mediated immune response. In some embodiments, the compositions and methods of the disclosure can elicit a humoral immune response. In some embodiments, the compositions and methods of the disclosure can elicit a systemic immune response. In some embodiments, the compositions and methods of the disclosure can elicit a mucosal immune response. In some embodiments, the compositions and methods of the disclosure can elicit an enhanced systemic immune response and an enhanced mucosal immune response.

In some embodiments, disclosed herein is a method of treating, preventing or inhibiting manifestation or symptoms an infection comprising administering a therapeutically effective amount of a composition or a pharmaceutical composition of the disclosure to a subject in need thereof.

In some embodiments, disclosed herein is a method of treating an infection comprising: (a) administering a therapeutically effective amount of a composition or a pharmaceutical composition of the disclosure to a subject in need thereof; wherein the infection is a Clostridioides Difficile (C. difficile) infection; (b) after the administering of the pharmaceutical composition, collecting a fecal sample from the subject; and (c) analyzing the fecal sample and determining a change in colony-forming units (CFU)/mg of C. difficile in feces a marker of infection.

In some embodiments, disclosed is a method of treating an infection, the method comprising administering to a subject in need thereof a therapeutically effective amount of a pharmaceutical composition comprising: (a) a cell-surface polysaccharide of Clostridioides Difficile (C. difficile); and (b) a polypeptide or polynucleotide encoding the polypeptide, wherein the polypeptide comprises a toxoid of C. difficile or a fragment thereof; wherein the administering is weekly or biweekly, and wherein the infection is caused by C. difficile.

In some embodiments, disclosed herein is a method comprising: (a) selecting a subject from a group consisting of: (i) a first subject, wherein the first subject is greater than 55 years of age; (ii) a second subject, wherein the second has a history of a C. difficile infection within a 6-month period; and (iii) a third subject, wherein the third subject has a positive C. difficile colony forming units (CFU)/mg count; and (b) administering a therapeutically effective amount of a pharmaceutical composition comprising a cell-surface polysaccharide of C. difficile; and a polypeptide or polynucleotide encoding the polypeptide, wherein the polypeptide comprises a toxoid of C. difficile or a fragment thereof.

In some embodiments, disclosed herein is a method of treating an infection, (a) identifying genetic sequence information of a biological sample obtained from a subject to determine a presence of the infection, wherein the infection is a Clostridioides difficile (C. difficile) infection; and (b) administering to the subject a therapeutically effective amount of a pharmaceutical composition comprising a cell-surface polysaccharide of C. difficile.

In some embodiments, disclosed herein is a method of treating an infection, the method comprising administering to a subject in need there of: (a) a therapeutically effective amount of a pharmaceutical composition of the disclosure; and (b) a therapeutic agent; wherein the infection is caused by Clostridioides difficile (C. difficile).

In some embodiments, disclosed herein is a method of treating an infection, the method comprising administering a pharmaceutical composition comprising a cell-surface polysaccharide of Clostridioides difficile (C. difficile), wherein the administering causes an immunoglobulin M (IgM) specific to the cell-surface polysaccharide to increase by at least about 250-fold compared to an absence of the pharmaceutical composition.

In some embodiments, the compositions and methods of the disclosure can elicit an enhanced T_{H}1 immune response or a T_{H}2 immune response. In some embodiments, an enhanced T_{H}1 response can comprise an increase in CTLs, increase in a cytokine associated with a T_{H}1 immune response, an increase in activated macrophages, an increase in NK activity, or an increase in the production of IgG2a. In some embodiments, the enhanced T_{H}1 immune response increases levels of a cytokine, for example, IL-2, IFN-γ, and TNF-0.

In some embodiments, a T_{H}1 immune response is elicited using a T_{H}1 adjuvant. In some embodiments, the T_{H}1 adjuvant can elicit increased levels of IgG2a production relative to immunization of the antigen without the T_{H}1 adjuvant. In some embodiments, the T_{H}1 adjuvant is a saponin formulation, virosome, virus-like particle, non-toxic derivatives of enterobacterial lipopolysaccharide (LPS), or immunostimulatory oligonucleotides (e.g., oligonucleotides comprising a CpG motif).

In some embodiments, a T_{H}2 immune response comprises an increase in a cytokine associated with a T_{H}2 immune response; or an increase in the production of IgG1, IgE, IgA and memory B cells. In some embodiments, the cytokine is IL-4, IL-5, IL-6, or IL-10. In some embodiments, the enhanced T_{H}2 immune response comprises an increase in IgG1 production.

In some embodiments, a T_{H}2 immune response can be elicited using a T_{H}2 adjuvant. A T_{H}2 adjuvant can elicit increased levels of IgG1 production relative to immunization of the antigen without adjuvant. In some embodiments, the T_{H}2 adjuvants is a mineral-containing composition, oil-emulsion, or an ADP-ribosylating toxin or a detoxified derivatives thereof. In some embodiments, the mineral-containing composition is an aluminum salt.

In some embodiments, the compositions of the disclosure comprise a T_{H}1 adjuvant and a T_{H}2 adjuvant, which elicits an enhanced T_{H}1 and enhanced T_{H}2 response. In some embodiments, a composition comprising a T_{H}1 adjuvant and a T_{H}2 adjuvant elicits an increase in the production of IgG1 and IgG2a. In some embodiments, the compositions of the disclosure comprising a T_{H}1 adjuvant and a T_{H}2 adjuvant can elicit an increased T_{H}1 or increased T_{H}2 immune response relative to immunization with a single adjuvant, for example, relative to immunization with a T_{H}1 adjuvant alone or immunization with a T_{H}2 adjuvant alone.

In some embodiments, the methods and compositions of the disclosure can increase the immune response (e.g., anti-PSII IgG) of a subject by about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, about 40-fold, about 41-fold, about 42-fold, about 43-fold, about 44-fold, about 45-fold, about 46-fold, about 47-fold, about 48-fold, about 49-fold, or about 50-fold.

The vaccine compositions of the disclosure can be administered therapeutically. Primary prophylaxis is the prevention or increased resistance to a disease that has not occurred. Secondary prophylaxis is a measure taken to prevent the recurrence of a disease that has already occurred. In some embodiments, a vaccine composition of the disclosure can be administered to a subject for primary prophylaxis, for example, to prevent CDI or increase resistance to CDI. In some embodiments, a vaccine composition of the disclosure can be administered to a subject for secondary prophylaxis, for example, to prevent a recurrent CDI.

In some embodiments, a vaccine composition of the disclosure can prevent symptoms of CDI. In some embodiments, a vaccine composition of the disclosure can ameliorate the symptoms of CDI. In some embodiments, a vaccine composition of the disclosure can reduce the incidence or severity of characteristic symptoms of CDI, for example, diarrhea, intestinal inflammation, necrosis of gastrointestinal tissues, weight loss, or fluid accumulation in the gut compared to a non-immunized subject.

### Patient Selection

In some embodiments, disclosed herein is a method of treating an infection, comprising: (a) selecting a subject from a group consisting of: (i) a first subject, wherein the first subject is greater than 55 years of age; (ii) a second subject, wherein the second has a history of a C. difficile infection within a 6-month period; and (iii) a third subject, wherein the third subject has a positive C. difficile colony forming units (CFU)/mg count; and (b) administering a therapeutically effective amount of a pharmaceutical composition comprising a cell-surface polysaccharide of C. difficile or a cell-surface polysaccharide conjugate; and a polypeptide comprising a toxoid of C. difficile or a fragment thereof. In some embodiments, a subject is at high risk for contracting CDI which include the elderly, adults with planned hospitalization, Long Term Care Facility residents and patients with co-morbidity requiring prolonged use of antibiotics.

### Combination Therapy

In some embodiments, the compositions and methods of the disclosure can be administered in combination with at least one additional agent for preventing or treating a CDI. In some embodiments, the at least one additional agent can be administered concomitantly with the composition or vaccine. In some embodiments, the at least one additional agent can be administered sequentially with the composition or vaccine. In some embodiments, the additional agent is an antibiotic, for example, vancomycin, fidaxomicin, metronidazole, or rifaximin. In some embodiments, the antibiotic is vancomycin. In some embodiments, the antibiotic is fidaxomicin. In some embodiments, the antibiotic is metronidazole. In some embodiments, the antibiotic is rifaximin.

### Companion Diagnostic

In some embodiments, disclosed herein is a method of treating an infection, the method comprising: (a) performing a real-time polymerase chain reaction (RT-PCR) or nucleic acid amplification test (NAAT) on a biological sample to determine a presence of the infection, wherein the infection is a C. difficile infection; and (b) administering to a subject in need thereof a therapeutically effective amount of a pharmaceutical composition comprising a cell-surface polysaccharide of C. difficile or a cell-surface polysaccharide conjugate and a polypeptide comprising a toxoid of C. difficile.

Various methods and assays for determining the presence of a CDI are suitable for use in the disclosure. Non-limiting examples of assays include polymerase chain reaction (PCR), quantitative PCR (qPCR), real-time PCR (RT-PCR), Sanger sequencing, restriction fragment length polymorphism (RFLP), microarray, Southern Blot, northern blot, western blot, eastern Blot, H&E staining, microscopic assessment of tumors, massively parallel sequencing (MPS), next-generation DNA sequencing (NGS) (e.g., extraction, purification, quantification, and amplification of DNA, library preparation), immunohistochemistry (IHC), protein quantification, chromogenic in situ hybridization (CISH), and fluorescent in situ hybridization (FISH).

### Kits

The present disclosure also provides kits. The kits include a vaccine composition of the disclosure in packaging, and written material that can include instructions for use, discussion of clinical studies, listing of side effects, and the like. Such kits can also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information can be based on the results of various studies, for example, studies using experimental animals involving in vivo models and studies based on human clinical trials. The kit can further contain another agent. In some embodiments, the vaccine composition of the present invention and the agent are provided as separate compositions in separate containers within the kit. In some embodiments, the vaccine composition of the present invention and the agent are provided as a single composition within a container in the kit. Suitable packaging and additional articles for use (e.g., measuring cup for liquid preparations, foil wrapping to minimize exposure to air, and the like) are known in the art and can be included in the kit. Kits described herein can be provided, marketed and/or promoted to health providers, including physicians, nurses, pharmacists, formulary officials, and the like. Kits can also, in some embodiments, be marketed directly to the consumer.

### EXAMPLES

### Example 1: Purification of PSII Polysaccharide

Step 1, Seed culture: A 2 mL frozen glycerol stock of C. difficile strain 8271 was thawed and used to inoculate 100 mL of PSII medium in a 500 mL baffled flask. The inoculated culture was incubated at 37° C. in an anaerobic environment for 16-18 hours until the culture reached an OD600 of 3.5-4.5.

Step 2, Fermenter (inoculation): 30 mL of the seed culture was used to inoculate a fermenter containing 10 L of PSII medium that had been sparged with nitrogen until the dissolved oxygen level was 0%.

Step 3, Fermentation (growth): The 10 L culture was grown at 37° C.±1.0° C. for 16-18 hours stirring at 225 rpm with constant nitrogen sparging. The cells were harvested when OD600 reached 4.0±0.5.

Step 4, Harvest: Cells were centrifuged by bucket centrifugation at 8000×g for 15 min at 22° C. The cells were washed in 1×PBS and spun again under the same conditions. The resulting cell paste was then frozen at less than −20° C.

Step 5, Cell Stripping: The cell paste was thawed at ambient temperature and resuspended in 4 mL/g of wet cell paste in 0.5% sodium deoxycholate prepared in phosphate buffered saline (PBS). The resuspended material was incubated at 60±2° C. for 24 hours shaking at 225 rpm. Following incubation, insoluble material was removed by centrifugation at 13,851×g for 15 minutes at 37° C. and supernatant collected.

Step 6, Ethanol Precipitation: Absolute (100%) ethanol was added to the supernatant from Cell Stripping to a final concentration of 20%. The solution was incubated at 4° C. for 60 minutes and then centrifuged at 15,000×g at 4° C. and the supernatant collected.

Step 7, Trichloroacetic Acid (TCA) Precipitation 1: 1 volume of TCA (6.12 M) was added to 4 volumes of the ethanol precipitation. The solution was incubated at 4° C. for 10 min. After the incubation with solution was centrifuged at 20,000×g for 15 minutes at 4° C. and the supernatant collected. The supernatant was filtered through a 0.45 μm filter.

Step 8, Ultrafiltration/Diafiltration (UFDF) 1: The supernatant from the TCA precipitation was diafiltered by tangential flow filtration (TFF) using a 1kDa molecular weight (MW) cut-off filter and 10 volumes of 10 mM sodium phosphate buffer (pH 7.0).

Step 9, Hydrophobic Interaction Chromatography on Phenyl Sepharose Column: Solid ammonium sulfate was added to PSII following UFDF 1 to a final concentration of 2 M. The pH of the solution was adjusted to 7.0, and the resulting solution was filtered through a 0.45 vim filter. The material was then loaded onto a a Phenyl Sepharose-Fast Flow column equilibrated with 10 mM sodium phosphate buffer and 2 M ammonium sulfate (pH 7.0). The column was washed with 3 column volumes of the equilibration buffer. The column flow through and wash were combined.

Step 10, UFDF 2: The combined flow though and wash from Step 9 were diafiltered by TFF using a 1kDa MW cut-off filter and 10 volumes of 10 mM sodium phosphate buffer (pH 8.0).

Step 11, Anionic Exchange Chromatography on Q Sepharose Column: The diafiltered PSII from Step 10 was applied to a Q Sepharose column equilibrated with 10 mM sodium phosphate buffer (pH 8.0). The column was washed with 5 CV of equilibration buffer and then eluted with 2.5 CV of 10 mM sodium phosphate buffer; 300 mM NaCl (pH 8.0).

Step 12, TCA Precipitation 2: 1 volume of TCA (6.12 M) was added to 4 volume of the Q-Sepharose column eluate. The solution was incubated at 4° C. for 10 min. After the incubation with solution was centrifuged at 20,000×g for 15 minutes at 4° C. and the supernatant collected. The supernatant was filtered through a 0.45 μm filter.

Step 13, UFDF 3: The filtered supernatant from Step 12 was diafiltered by TFF using a 1kDa MW cut-off filter and 10 volumes of water.

Step 14, Final Concentration on 3 kDa Filter: The PSII from Step 13 was applied to 3 kDa MW cut-off spin filters and concentrated by centrifugation at 8000×g for 30 minutes at 4° C. The retained PSII was washed 3 times with water by repeating centrifugation step. The final material was aliquoted and stored at −20° C.

Analysis of Polysaccharide Content in Purified PSII: Polysaccharide content of the PSII was determined by a colorimetric assay (Anthrone or Resorcinal).

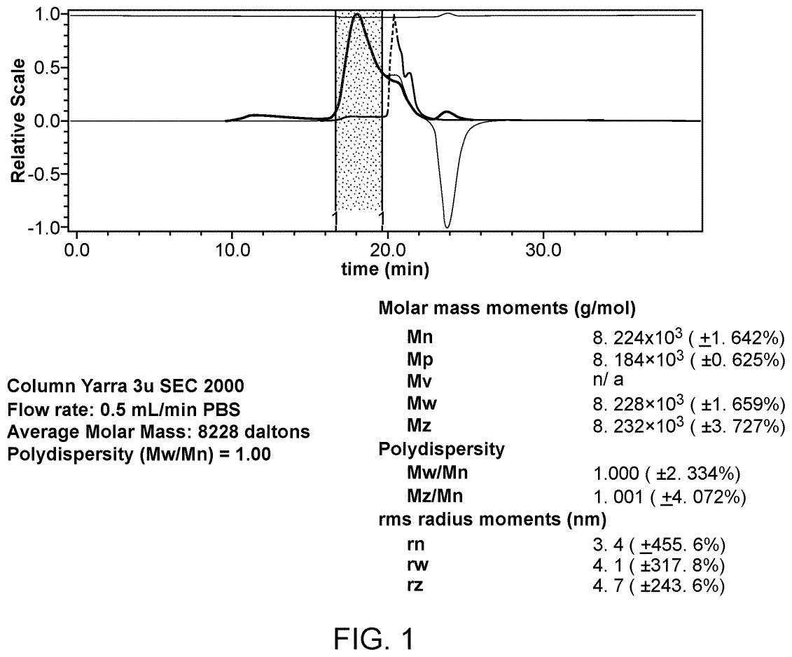

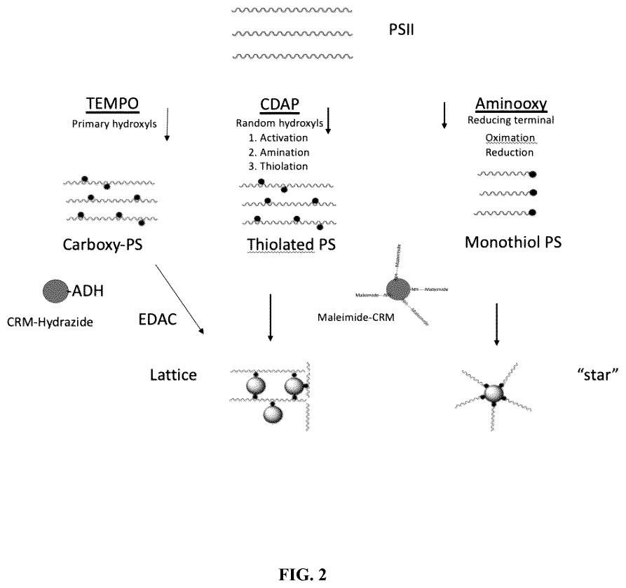

Determination of MW by size exclusion chromatography and multi-angle laser-light scattering (SEC-MALS): The MW and polydispersity of PSII was analyzed by size exclusion chromatography multiangle light scattering (SEC MALS). Using a do/dc of 0.137, the average MW of the PSII was determined to be 8.8 kDa, with extremely low polydispersity. The MW was rounded up to 10 kDa to calculate the molar ratio of PSII:CRM. FIG. 1 shows the SEC MALS analysis of PSII and the corresponding parameters.

The Monosaccharide Composition of the Purified PSII following acid hydrolysis was determined using high performance anionic exchange with pulsed amperometric detection (HAPAE-PAD). The results showed glucose, N-acetyl galactosamine, mannose at a ratio of 3:2:1 consistent with know composition of PSII. Only minor amounts of N-acetyl glucosamine were observed in the analysis.

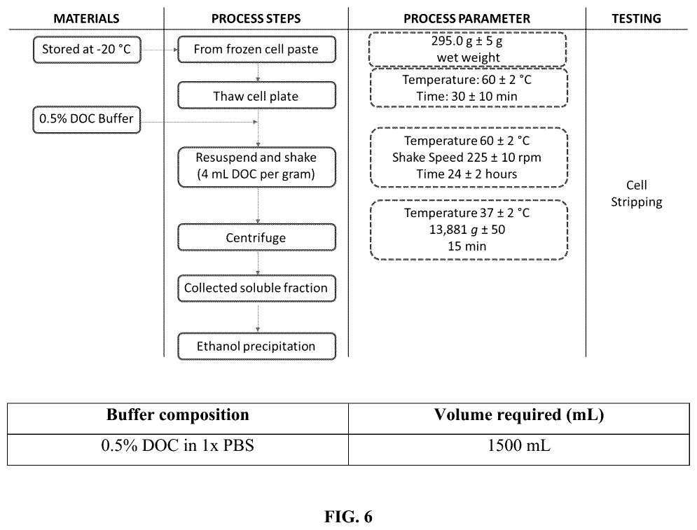

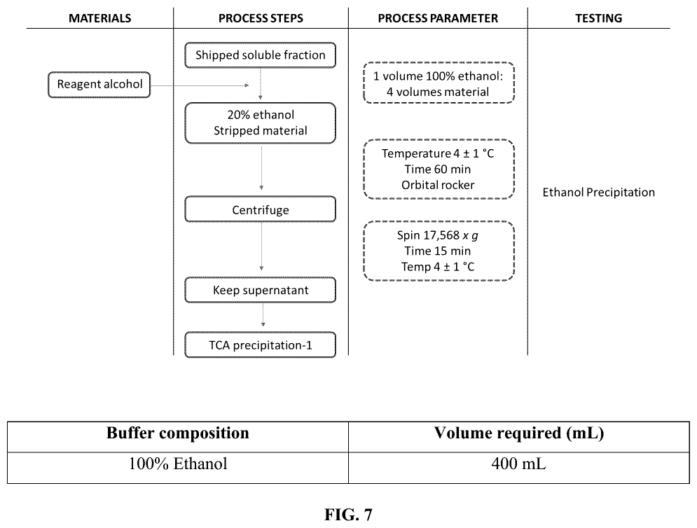

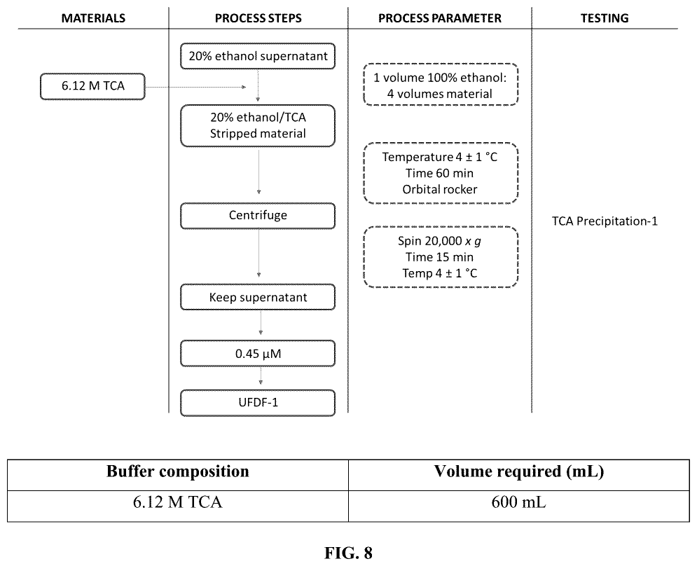

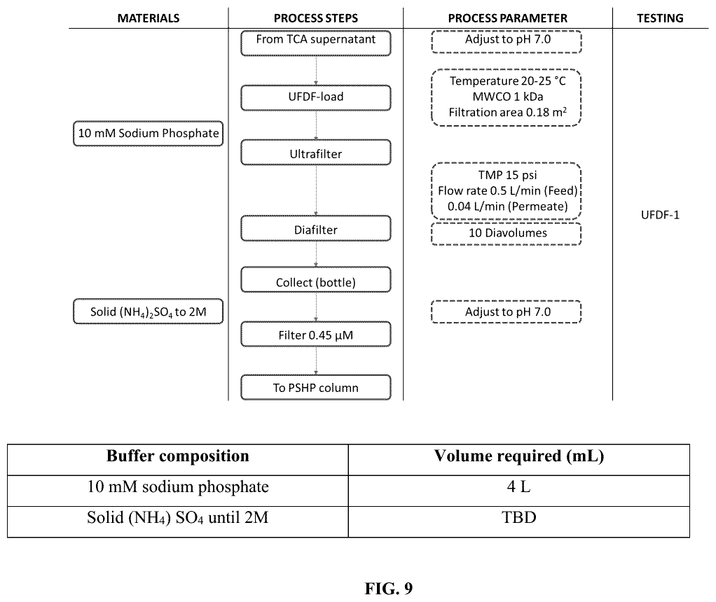

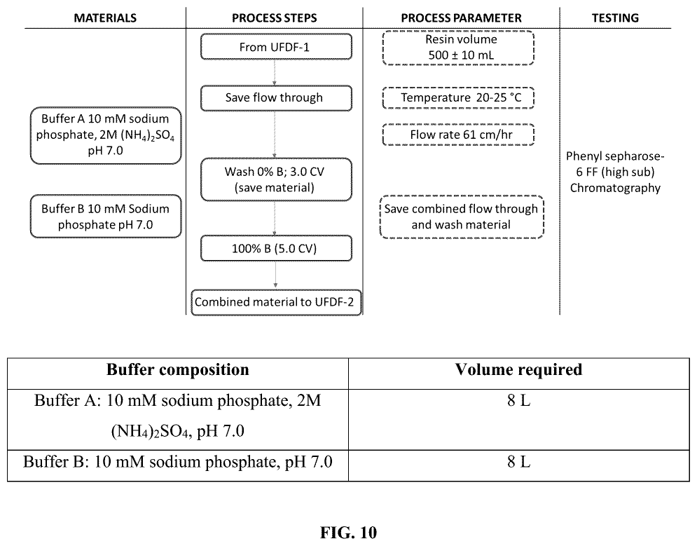

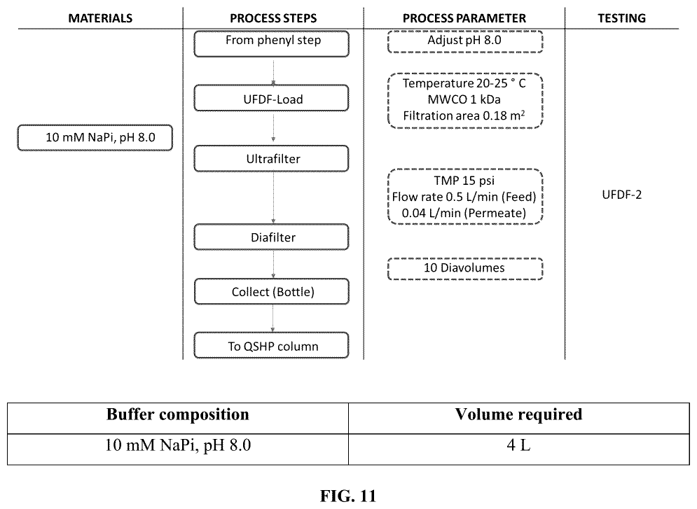

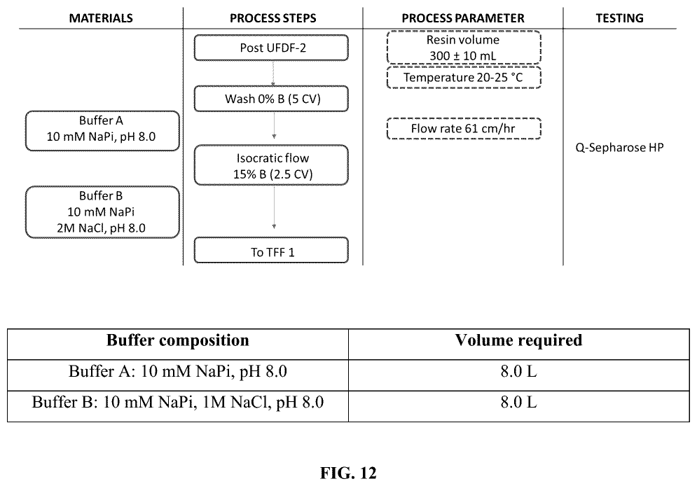

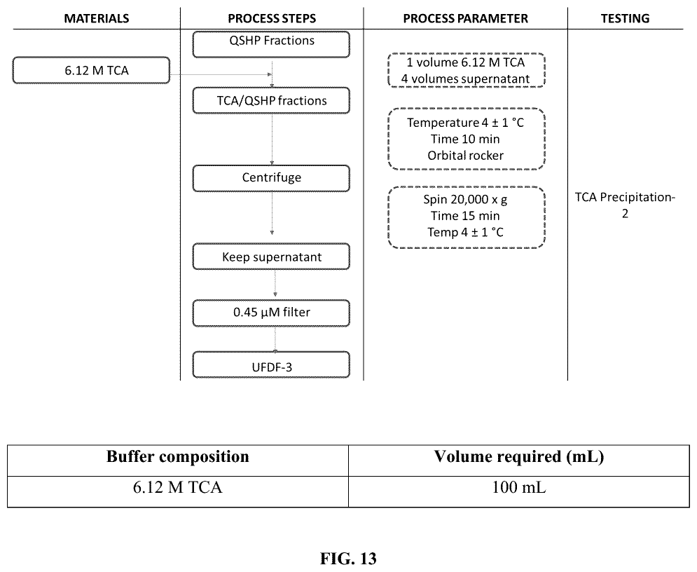

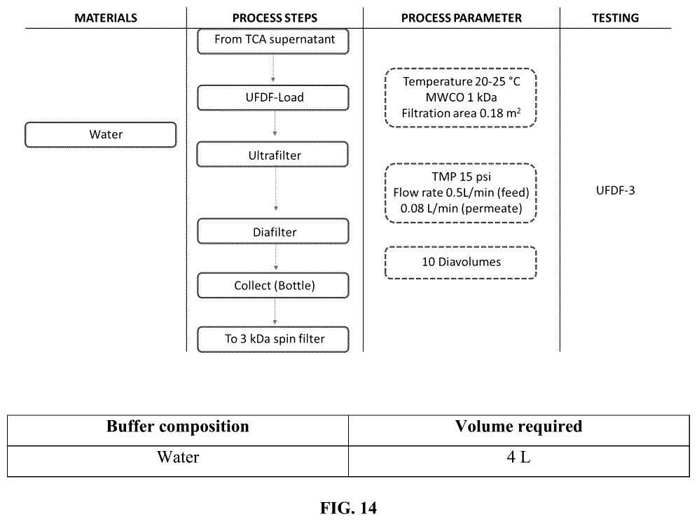

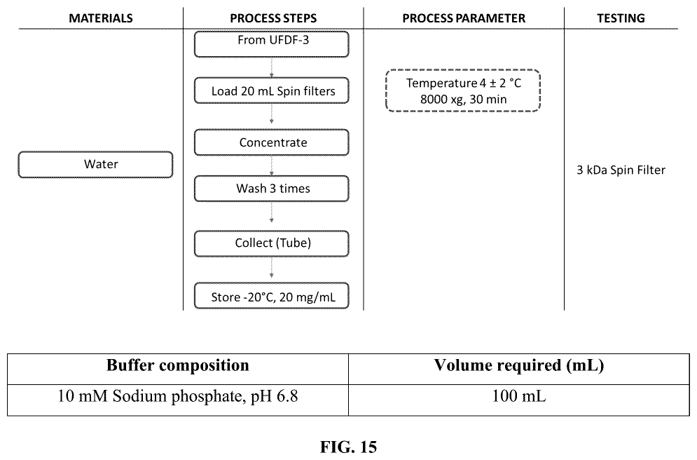

FIG. 6 outlines the steps for stripping C. difficile cells using sodium deoxycholate (DOC) for PSII purification. FIG. 7 outlines the procedure for the 20% ethanol extraction and precipitation stage of PSII purification. FIG. 8 outlines the procedure for the first TCA precipitation of the 20% ethanol extracted material for purification of PSII. FIG. 9 outlines the procedure for ultrafiltering/diafiltering (UFDF 1) PSII after the first TCA precipitation, and addition of ammonium sulfate in preparation for the hydrophobic interaction chromatography stage on a Phenyl Sepharose column. FIG. 10 outlines the procedure for applying the UFDF 1 material onto the Phenyl Sepharose column and collection of the flow through and wash fractions. FIG. 11 outlines the procedure for the second UFDF (UFDF-2) step of pooled fractions from the Phenyl Sepharose column in preparation for separation by anionic exchange chromatography on a Q-Sepharose column. FIG. 12 outlines the procedure for applying the UFDF-2 material on the Q-Sepharose column and eluting the PSII using 10 mM sodium phosphate buffer containing 300 mM NaCl (15% of buffer B). FIG. 13 outlines the procedure for the second TCA precipitation of the Q-Sepharose column eluate. FIG. 14 outlines the procedure for the ultrafiltration/diafiltration (UFDF-3) of the PSII following the second TCA precipitation into water. FIG. 15 outlines the procedure for concentration the material from UFDF-3 on a 3 kDa MW cut-off spin filter. TABLE 3 outlines the procedure for PSII purification.

TABLE 3

| Step number | Procedure |
|---|---|
| 1 | Cell stripping |
| 2 | Centrifugation |
| 3 | Ethanol precipitation |
| 4 | Centrifugation |
| 5 | TCA precipitation-1 |
| 6 | Centrifugation |
| 7 | UFDF-1 |
| 8 | Phenyl sepharose-6 FF (high sb) |
| 9 | UFDF-2 |
| 10 | Q-Sepharose HP |
| 11 | TCA Precipitation-2 |
| 12 | UFDF-3 |
| 13 | 3 kDa spin filter |
| 14 | Store −20° C.; 20 mg/mL |

### Example 2: Conjugation Chemistry of PSII-Conjugates

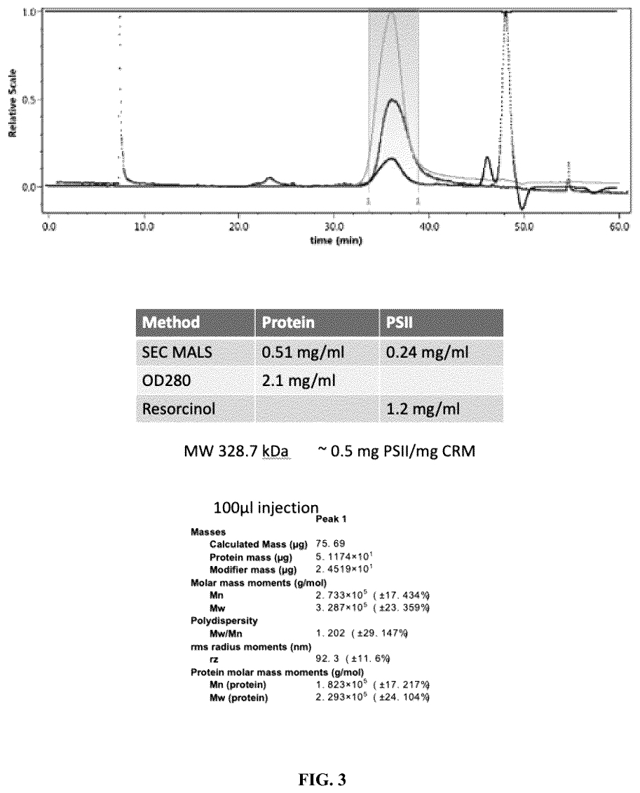

Four different PSII-CRM₁₉₇ conjugates were prepared using periodate-mediated reductive amination chemistry (RedAm), cyanylation chemistry (CDAP), TEMPO oxidation-amination chemistry (TEMPO), and end-linked aminooxy thiol click chemistry (End-Linked). With the RedAm, CDAP, and TEMPO chemistries, multiple points of attachment could be made between the PSII and CRM₁₉₇, resulting in high molecular weight conjugates that formed “lattice” structures (FIG. 2). With the End-linked chemistry, only a single point of attachment was made between the PRI chain and CRM₁₉₇, resulting in a conjugate with a “star” structure (FIG. 2).

Preparation of derivatized CRM₁₉₇: EcoCRM® CRM₁₉₇ was used for all conjugation methods. Hydrazide- or maleimide-derivatized CRM₁₉₇ was used, depending on the conjugation protocol.

Preparation of CRM₁₉₇-ADH: A 10 mg/mL solution of EcoCRM® was diluted to 4 mg/mL into 0.1 M MES buffer at pH 6.0 and combined with an equal volume of 0.5 M adipic acid dihydrazide (ADH) in 0.1 M MES, pH 6. The solution was then prepared to 5 mg/mL EDAC from a 100 mg/mL stock. After 2 hr, the pH of the solution was raised to 8, and the reagents were removed by dialysis. The hydrazide ratio was determined using a TNBS assay and found to be 6 ADH/CRM. The desired product was obtained at about a 70% yield. CRM-ADH, like other ADH-derivatized proteins, stuck to SEC HPLC columns and did not provide clean chromatograms. SDS-PAGE analysis showed a single band for CRM-ADH (data not shown).

Preparation of CRM-maleimide: EcoCRM® at 5 mg/mL was labeled with 25× N-γ-maleimidobutyryl-oxysuccinimide ester (GMBS) at pH 7.2 for 1 hr, desalted using a Vivaflow 10 kDa tangential flow filtration device, and buffer exchanged into 10 mM sodium phosphate/10 mM EDTA, pH 6.8+5% sucrose. The CRM-maleimide product was aliquoted and stored at −80° C. until use. The maleimide ratio was determined by measuring the reaction of cysteine thiol, and averaged 15 maleimide/CRM. The typical yield for the reaction was greater than 80%.

Preparation of PSII polysaccharide: Crude PSII was solubilized in DI water to make a 50 mg/mL solution. The volume of the PSII solution was reduced by half using a 3KD amicon ultra4 spin device. The solution was brought back to the original volume using DI water, and the volume was reduced again to half by spinning at 4000 RPM. The process was repeated 4-5 times, and the resulting solution was lyophilized to obtain pure PSII. Alternatively, a 100 mg/mL solution of the crude PSII was prepared in DI H₂O. The solubilized PSII material was purified and lyophilized using a 7KDa Zeba column procedure to provide a clean product. The amount of pure PSII collected using this protocol varied in yield between 50-75% of the starting crude mass.

Synthesis of PSII-CRM₁₉₇ Conjugates: Conjugates of C. difficile PSII capsular polysaccharide with the carrier protein CRM₁₉₇ were prepared. (2,2,6,6-Tetramethylpiperidin-1-yl)oxyl (TEMPO)-mediated oxidation and 1-cyano-4-dimethylaminopyridinium tetrafluoroborate (CDAP) chemistry were used to functionalize repeat units along the polymer chain. TEMPO-oxidized PSII resulted in carboxyl groups that were subsequently conjugated to hydrazide-derivatized CRM₁₉₇ using 1-ethyl-3-(3-dimethylaminopropyl)carbodiimide (EDAC). The CDAP-activated PSII was derivatized with amines, thiolated, and linked to maleimide-derivatized CRM₁₉₇. A third method used reductive amination chemistry to derivatize the end of the polymer with a single thiol group. Thiolated PSII was then conjugated to maleimide-derivatized CRM₁₉₇. TEMPO and CDAP activated multiple hydroxyl groups and resulted in lattice conjugates. The third conjugation method via the terminal reducing sugar did not cause crosslinking, resulting in a lower MW conjugate. FIG. 2 illustrates an overview of TEMPO, CDAP, and aminooxy conjugation chemistry schemes.

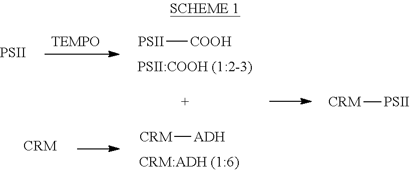

A) PSII TEMPO Oxidation/EDC Conjugation: SCHEME 1 shows the TEMPO route for PSII-CRM₁₉₇ conjugation. PSII was solubilized at a concentration of 10 mg/mL in water. To the solution were added 30 μL of TEMPO (10 mg/mL in water) per mL of the PSII solution, followed by 6.25 μL NaBr (100 mg/mL in water). The pH of the mixture was then increased to was added and the pH raised to 9.0 with 0.1 M NaOH. The resulting solution was chilled, and 2.5 μL of 14.5% sodium hypochlorite was added per mL of the solution. The pH of the solution was maintained at 9 for 1.5 hours, and 25 μL of ethanol per mL of the solution was added. The resulting mixture was stirred for 15 min. The solution was diluted with water, desalted into water using an Amicon Ultra 4 centrifugal device (3 kDa cutoff), and lyophilized. The extent of oxidation was estimated by EDAC amination to obtain an amine:PSII ratio of 3-4 per PSII.

EDAC Conjugation of PSII CRM-ADH: PSII-COOH (10 mg/mL) and CRM-ADH (˜8.2 mg/mL) were mixed at a ratio of 1.6:1. 1M MES buffer pH 6 was added to obtain a final concentration of 0.1 M MES buffer and pH 6. EDC solubilized in a minimum amount of water was added to the solution to obtain a 10 mg/mL final concentration in the reaction mixture. The reaction mixture was reacted for 2 hr at room temperature and reacted further overnight (15 hours) at 4° C. The conjugate was purified using a Superdex 200 column (1.6 diameter, 60 cm height). The early eluting fractions were pooled and concentrated using an Amicon pressure cell device with a 30 kDa membrane. Protein concentration was determined by measuring absorbance at 280 nm, and the amount of PSII was estimated using a resorcinol method. The final concentration of PSII was ˜1.2 mg/mL; CRM₁₉₇ 2.1 mg/mL; 0.57 mg PSII/mg CRM; PSII:CRM₁₉₇ ratio 3:1 to 4:1. The resulting conjugate was analyzed by SEC MALS, but showed very poor recovery on the HPLC column. FIG. 3 TOP PANEL shows the SEC MALS trace for the TEMPO/EDC/CRM₁₉₇ conjugate. BOTTOM PANEL shows the analytical summary of the TEMPO/EDC/CRM₁₉₇ conjugate.

B) PSII CDAP Thioether conjugation: SCHEME 2 shows an overview of PSII-CRM₁₉₇ conjugation using CDAP conjugation.

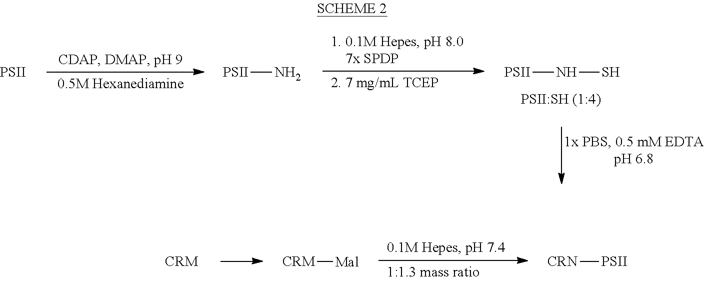

PSII amination: PSII was solubilized at a concentration of 20 mg/mL in saline. 200 μL/mL of 2.5 M DMAP was added to the solution, and the pH of the solution was adjusted to pH 9 in an ice bath. Activation was started by adding 100 μL CDAP per mL of the solution using a 100 mg/mL stock solution in acetonitrile. The pH of the solution was maintained at 9 using 0.1 M NaOH. After 10 min, 500 μL of 0.5 M 1,6-hexanediamine was added per mL of the solution. After 1 hr at 4° C., the PSII solution was desalted using a Zeba column, and the eluent was lyophilized. The extent of amination was determined using TNBS, and the results afforded 3-4 amines per PSII.

PSII-NH2 thiolation: PSII-NH2 was solubilized at a concentration of 3.5 mg/mL in 0.1M HEPES, pH 8. SPDP was added in excess from a 0.1 M stock solution prepared in DMSO. After 1.5 hr, the product was deprotected with TCEP and desalted into PBS+5 mM EDTA, pH 6.8. The DTNB assay indicated the presence of ˜3-4 thiols per PSII. The reaction yield efficiency was 80%.

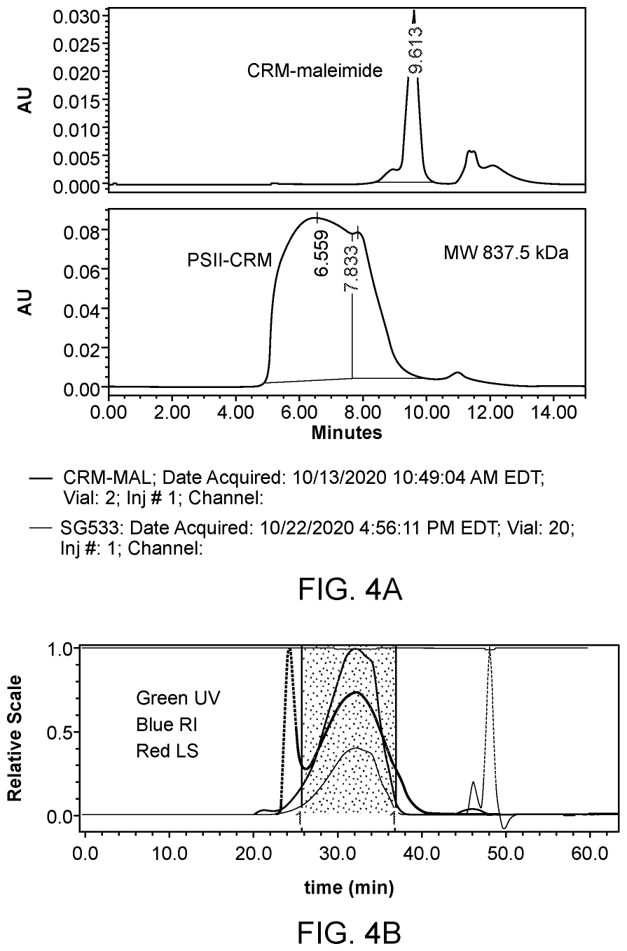

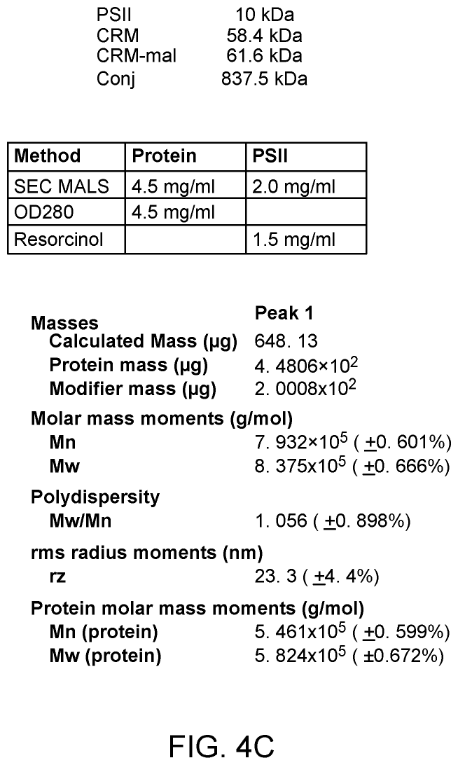

PSII-SH CRM-maleimide conjugation: PSII-SH and CRM-maleimide were combined at a ratio of 1.3:1 by mass. The conjugate was purified on a Superdex 200 column (1.6 diameter, 60 cm height) equilibrated with PBS. The purified material was concentrated using an Amicon stirred pressure cell with a 30 kDa membrane. The conjugate was analyzed by SEC, SEC MALS, resorcinol and OD 280. The yield of the conjugation calculated based on the PSII mas sin the conjugate was 33%. FIG. 4A show CRM-maleimide and PSII-CRM₁₉₇ SEC MALS traces. FIG. 4B shows a SEC trace of the PSII-CRM₁₉₇ CDAP conjugate. FIG. 4C shows the analytical parameters used to analyze the PSII-CRM₁₉₇ CDAP conjugate.

C) End-linked PSII: SCHEME 3 shows an overview of PSII-CRM₁₉₇ conjugation via the reductive amination route.

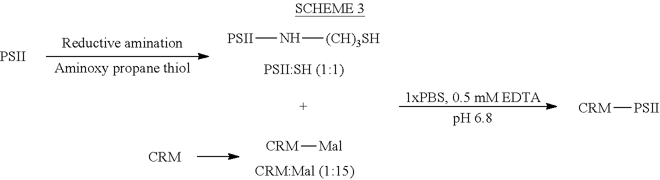

Reductive amination reaction/End thiolation of PSII: PSII was solubilized at a concentration of 5 mg/mL in DMSO. 5 equivalents of 1-aminooxy-3-thiol-propane was added After mixing the resulting solution for 30 min, 6 equivalents of sodium triacetoxyborohydride was added. The reaction was stirred further for 18 h at 50° C. The resulting mixture was then cooled, and the PSII was precipitated with ethanol. The precipitate was collected by centrifugation. The pellet was resolubilized in water and further purified with an Amicon Ultra-4 3 kDa cutoff centrifugal device and lyophilized. The reaction yield was 85%.

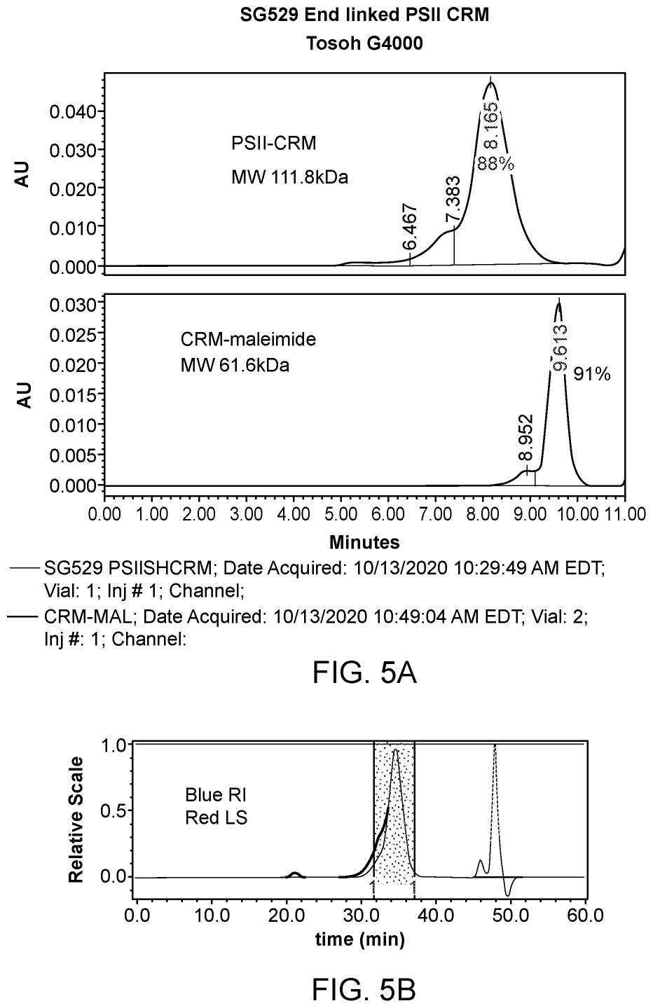

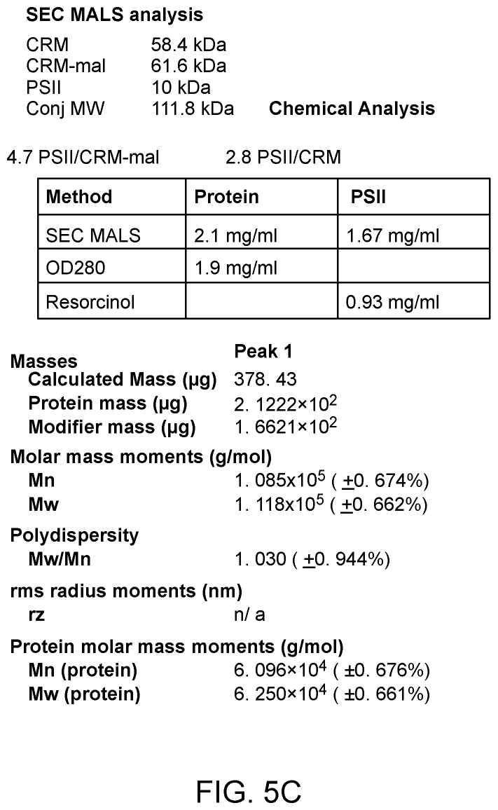

PSII end-linked thiol-maleimide-CRM 197 conjugation: The PSII thiol was solubilized at a concentration of about 10 mg/mL in DI water, and TCEP was added to reach a final concentration of 6 mg/mL. After a 1 hr incubation, the product was purified with an Amicon Ultra-4 3K centrifugal device, followed by desalting on a Zeba spin column (7 kDa cutoff) into a final buffer of PBS+EDTA, pH 6.8. The thiol was assayed with DTNB, which indicated about 1 thiol/PSII polymer. CRM-maleimide (15 maleimide/CRM) at a concentration of 5 mg/mL was combined with about 8 thiols per CRM. After an overnight reaction, the conjugate was purified on a Superdex200 column equilibrated in PBS. The purified conjugate was concentrated using an Amicon stirred pressure cell with a 10 kDa membrane. The conjugate was analyzed by SEC, SEC MALS, resorcinol and OD 280. FIG. 5A shows PSII-CRM₁₉₇ and CRM-maleimide SEC MALS traces. FIG. 5B shows a SEC trace of PSII-CRM. FIG. 5C shows the analytical parameters of the SEC MALS analysis, OD280, and resorcinol assay.

TABLE 4 shows a summary of PSII-CRM₁₉₇ conjugate characteristics.

TABLE 4

| | Activated | # of active | Conjugate |
|---|---|---|---|
| Chemistry | group | groups/PSII | form |
| TEMPO/EDC | Primary | 3-5 carboxyls | Lattice |
| | hydroxyl | | |
| CDAP/Thioether | Hydroxyls | 3-5 thiols | Lattice |
| Reductive | Reducing end | 1 thiol | Star |
| oxidation/thioether | | | |

Carbohydrate assay: A resorcinol/sulfuric acid method was used to assay for carbohydrate moieties. Initially, purified PSII with a standard solution prepared by dry weight was used for the assay. Due to the uncertainty of preparing an accurate solution of the PSII standard, a mix of N-Ac-Gal:Glu:Man 2:2:1 (proportion found in PSII repeat group) and glucose only were evaluated. Both the N-Ac-Gal:Glu:Man 2:2:1 mixture and glucose only resulted in similar standard curves. Glucose was used as the standard in the carbohydrate assay.

Amine and hydrazide assays: 2,4,6-Trinitrobenzenesulfonic acid (TNBS) was used to measure amines and hydrazides using glycine or adipic dihydrazide, respectively.

Thiol assay: Thiols were measured using ‘,5’-dithiobis-(2-nitrobenzoic acid) (DTNB).

TABLE 5 shows the PSII conjugates prepared using the three difference chemistries.

TABLE 5

| | PSII | CRM₁₉₇ | | | | | | | MW |
|---|---|---|---|---|---|---|---|---|---|
| Chemistry | (mg) | (mg) | Mg/mg | Mole/mole | vol | | Mg PSII | | (kDa) |
| TEMPO EDC | 1.2* | 2.1 | 0.57 | 3.3 | 5 | mL | 6 | mg | 328.7 |
| | 1.5** | | 0.7 | | | | 7.5 | mg | |
| CDAP thioether | 2*** | 4.5 | 0.44 | 2.6 | 7.5 | mL | 15 | mg | 837.5 |
| Oxime/thioether | 1.7*** | 1.9 | 0.89 | 5.2 | 7.5 | mL | 12.75 | mg | 111.8 |

*resorcinol assay, glucose standard
**1.25x correction for under-estimation by glucose
***Determined by SEC MALS

### Example 3: Prophylactic Immunization and Challenge with C. difficile

The PSII-CRM₁₉₇ conjugates were made with reductive amination (RedAM), TEMPO, or CDAP conjugation chemistry using the methods of EXAMPLE 1. The conjugates were used to immunize mice at a 20 μg dose level using a 3-biweekly dose regimen followed by an intragastric (IG) challenge with 10⁴ colony-forming unit (CFU) C. difficile spores. In the prophylactic immunization and challenge model, 3 of 5 mice immunized with the CDAP conjugate succumbed to a C. difficile infection. No mice died in the groups immunized with TEMPO and RedAM conjugates, but no appreciable protection was observed against the C. difficile challenge.

Serum sample harvest: Mouse blood was collected into serum separator tubes via puncture of the submandibular vein using sterile Goldenrod animal lancets prior to first immunization (pre-immune), day 14 (˜2 weeks after the first immunization), day 28 (˜2 weeks after the second immunization), and day 42 (˜2 weeks after the third immunization). Blood tubes were stored up to 16-18 hours at 2-8° C. before serum collection. Following storage, blood was warmed to ambient temperature and then centrifuged at 10,000 rpm for 10 minutes in the microfuge. The serum above the gel clot was collected into labeled microcentrifuge tubes. Pooled and individual serum samples were stored at −20° C. until use in the assay.

Fecal pellet harvest: Fecal pellets (1 pellet≈˜25 mg) were collected at designated timepoints into pre-weighted sterile Eppendorf tubes. 500 μL of sterile PBS+20% glycerol was added to each fecal sample before homogenization. After homogenization, 60 μL of fecal slurry was aliquoted into 2 sterile Eppendorf tubes and frozen until ready for plating. The remainder of the fecal slurry was kept frozen until the sample was ready for fecal DNA extraction.

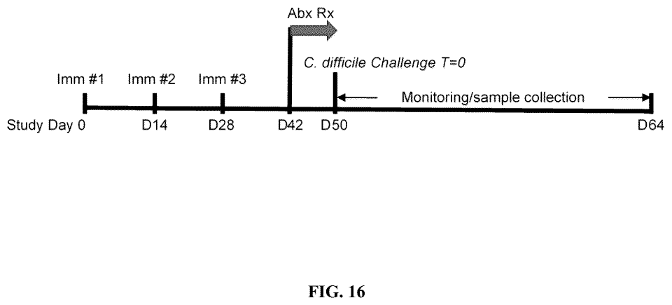

C. difficile prophylactic immunization and challenge mouse model of CDI: Mice were immunized intramuscularly 3 times at biweekly intervals with the test articles. Serum samples were collected to evaluate immune responses prior to each immunization. Following completion of the 3-biweekly dose regimen, the mice were administered an antibiotic cocktail of kanamycin (0.4 mg/mL), gentamicin (0.035 mg/mL), colistin (850 U/mL), metronidazole (0.215 mg/mL), and vancomycin (0.045 mg/mL) in drinking water for 5 days. A 1:5 dilution of the antibiotic cocktail was also administered IG to animals 3 times during the treatment period to ensure each animal received antibiotics prior to the challenge. On the fifth day, mice were given regular drinking water. One day prior to the C. difficile challenge, mice were weighed, and the weights were averaged to prepare a solution of clindamycin so each mouse was IP administered 10 mg/kg clindamycin. The next day (challenge day 0), each mouse was weight, fecal pellets were harvested, and ˜10⁴ CFU C. difficile spores in 100 μL PBS were administered by IG. Mice were monitored for symptoms and weight loss post-infection. Periodically, fecal pellets were harvested for evaluation of CFU outgrowth over time. Terminal blood collection was performed on any remaining animals on the last day of the study. FIG. 16 shows a general schematic for prophylactic immunization and challenge mouse model of CDI. TABLE 6 shows the Anti-PSII IgG responses at Day 42. The data show that the PSII-CRM₁₉₇ conjugates elicited anti-PSII IgG antibodies in C. difficile-primed and naïve mice.

TABLE 6

| | Anti-PSII IgG Antibody Titers at Day 42 | |
|---|---|---|
| | Unprimed | C. difficile- |
| Test article | (Naïve mice) | primed mice |
| CDAP PSII-Conjugate | 6400 | 32000 |
| RedAm PSII- Conjugate | 800 | 1600 |

### Example 4: Secondary Prophylaxis as Determined by Fecal Colonization and Antibody Response

A secondary prophylaxis model of CDI was used to determine if mice that were immunologically primed from a primary CDI had a boost in immune response upon vaccination with a PSII conjugate vaccine. Groups of mice were treated with an antibiotic cocktail prior to C. difficile infection by oral gavage. The mice were monitored for morbidity, mortality, weight loss, and other CDI symptoms over 2-3 weeks. Infected mice, or C. difficile-primed mice, were allowed to recover until the mice reached normal weight. Then, baseline blood collection, weight, and fecal samples were obtained from each mouse prior to initiation of the 3-biweekly dosing regimen.

PSII-CRM₁₉₇ conjugates made with RedAM or CDAP were used to immunize mice at a 20 μg dose level using a 3-biweekly dosing regimen. Fecal samples were harvested weekly, and blood samples were collected prior to each immunization in the 3-biweekly dosing regimen. The primary study endpoints were: 1) Fecal CFU on days 0, 14, 28, 56, and beyond; and 2) PSII IgG responses on days 0, 14, 28, 42, 56, and beyond. Serum samples and fecal pellets were harvested as described in EXAMPLE 3, described above.

Mice were administered an antibiotic cocktail of kanamycin (0.4 mg/mL), gentamicin (0.035 mg/mL), colistin (850 U/mL), metronidazole (0.215 mg/mL), and vancomycin (0.045 mg/mL) in drinking water for 5 days. A 1:5 dilution of this antibiotic cocktail was administered IG to animals 3 times during the treatment period to ensure each animal received antibiotics prior to the challenge. On the fifth day, mice were returned to regular drinking water. One day prior to the C. difficile challenge, the mice were weighed. The weights were averaged to prepare a solution of clindamycin such that each mouse was administered 10 mg/kg clindamycin by IP route. The next day (Challenge Day 0), each mouse was weighed, fecal pellets were harvested, and ˜10⁴ CFU C. difficile spores in 100 μL, PBS were administered to each mouse by IG route. Mice were monitored for morbidity, mortality, symptoms, and weight loss post-infection. Periodically, fecal pellets were harvested for evaluation of CFU colonization. When mice recovered body weight following the primary infection, the 3-biweekly dosing regimen was initiated in which mice were immunized IM 3 times at a biweekly interval with the test articles. Serum samples were collected prior to each immunization to evaluate immune responses, and fecal pellets were collected prior to each immunization to evaluate fecal colonization.

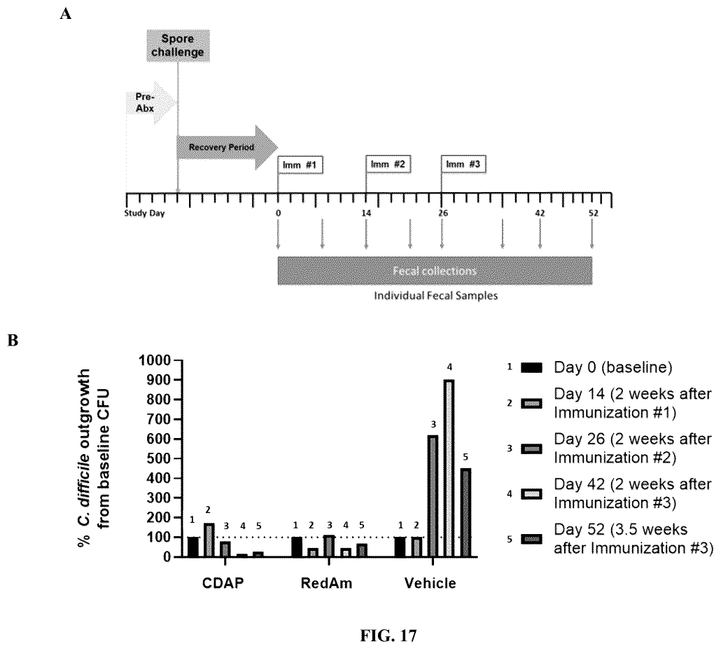

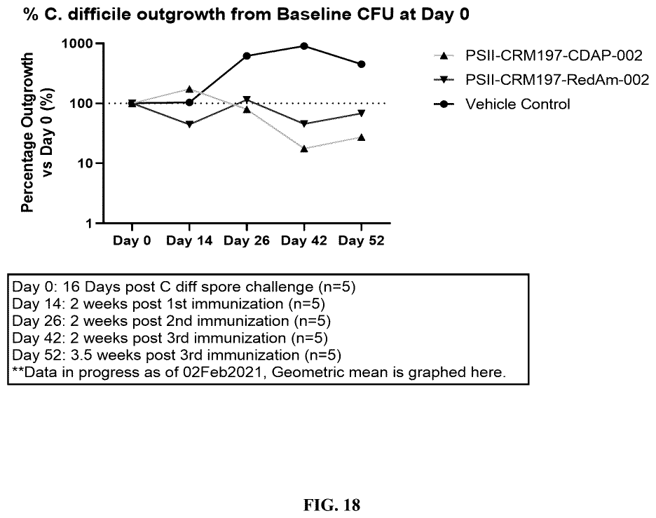

FIG. 17 PANEL A shows the timeline of the spore challenge, immunization schedule, and fecal collection time points of the individual fecal samples. PANEL B shows % C. difficile geometric mean outgrowth following therapeutic immunization, normalized to baseline CFU at Day 0 prior to immunization. FIG. 18 shows % C. difficile geometric mean outgrowth from baseline CFU at Day 0. The data show that vaccination with both PSII-CRM₁₉₇ conjugates controlled the outgrowth of C. difficile compared to the vehicle control in C. difficile-primed mice.

PSII-CRM₁₉₇ conjugates were tested for the ability of the conjugates to boost C. difficile-primed mouse antibody responses. PSII-CRM₁₉₇ conjugates were used to immunize naïve mice and mice “primed” with C. difficile spores. The mice were treated with 3-biweekly intramuscular immunizations. Anti-PSII IgG antibody levels in sera were determined 2 weeks following the second immunization (Day 28) and the third immunization (Day 42). The data shows that the PSII-CRM-197 conjugate elicited an 8-fold increase in titer anti-PSII IgG antibodies in C. difficile-primed mice 2 weeks following the second immunization (Day 28) compared to the response in naïve mice. Naive mice administered the 10 μg dose level of PSII-CRM₁₉₇ conjugate had a 4-fold lower response at Day 42 (2 weeks following completion of the 3-biweekly dosing regimen) than the C. difficile-primed mice administered the same dose level. However, the Day 42 anti-PSII IgG antibody response of naïve mice administered the 25 μg dose level of PSII-CRM₁₉₇ conjugate was comparable to the anti-PSII IgG response elicited by either 10 μg or 25 μg dose level of C. difficile-primed mice.

TABLE 7

Anti-PSII IgG GMT

| Mice | Vaccine | Dose | Day 28 | Day 42 |
|---|---|---|---|---|
| Naïve | PBS | None | <50 | <50 |
| | PSII | 25 μg | <50 | <50 |
| | PSII- CRM₁₉₇ | 25 μg | 1600 | 25600 |
| | | 10 μg | 800 | 6400 |
| C. difficile | PBS | None | <50 | <50 |
| primed | PSII | 25 μg | <50 | <50 |
| | PSII-CRM₁₉₇ | 25 μg | 12800 | 51200 |
| | | 10 μg | 6400 | 25600 |

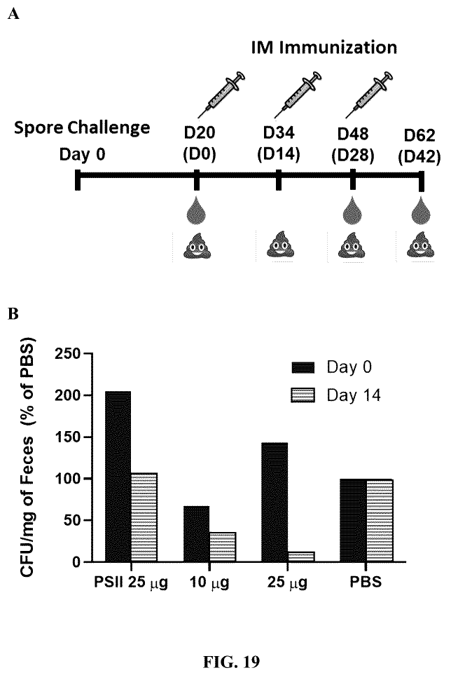

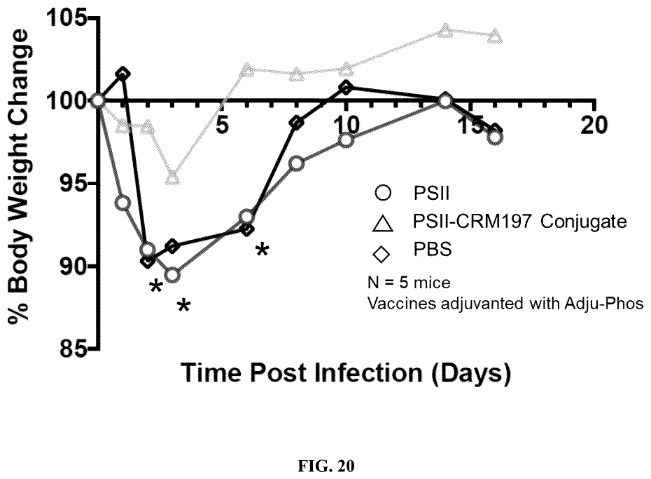

FIG. 19 PANEL A illustrates the immunization schedule used to obtain Anti-PSII IgG data. PANEL B shows fecal colonization (CFU/mg of feces) determined 2 weeks after the first immunization treatment on day 14. FIG. 20 shows that prophylactic PSII-CRM₁₉₇ conjugate immunization protected mice from weight loss and symptoms. Statistically significant weight loss was observed from T=0 in PSII and PBS-immunized mice. No significant weight change was observed for mice immunized with the PSII-CRM₁₉₇ conjugate.

### Example 5: Immunogenicity of PSII-CRM₁₉₇ Conjugates in Mice

PSII-CRM₁₉₇ conjugates prepared with different chemical methods were evaluated in murine models of C. difficile infection (CDI) looking at both immunogenicity and protection. A series of murine studies were performed comparing immunogenicity and protection with conjugates prepared with CDAP, TEMPO, and End-linked chemistries. Based on immunogenicity and protection data in a murine model of CDI as well as ease of manufacturing, the end-linked chemistry was chosen for further development.

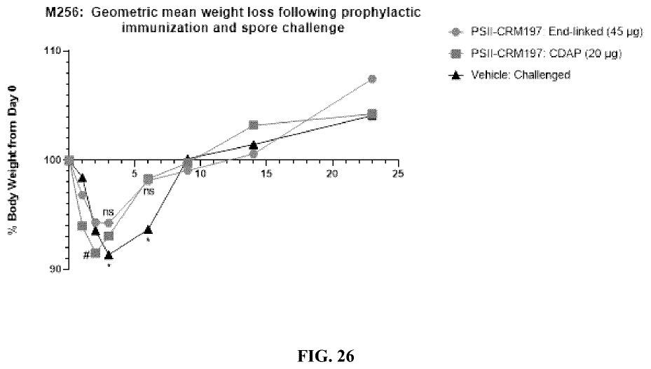

Administration of 45 μg of the end-linked PSII-CRM₁₉₇ immunizations (adjuvanted with 1 mg/mL AlPO₄ adjuvant in sterile saline) protected mice from significant body weight loss against a C. difficile spore challenge. In contrast, 20 μg of the CDAP PSII-CRM₁₉₇ immunization provided partial protection against a C. difficile spore challenge. FIG. 26 shows the geometric mean weight loss following prophylactic immunization and spore challenge. # indicates significant weight loss at day 2 vs day 0 for the CDAP-immunized group. * indicates significant weight loss at day 3 and 6 for the control group.

TABLE 8 shows the weight loss % by day after day 0 upon immunization with the test articles. TABLE 9 shows anti-PSII IgG ELISA Titers on days 0, 14, 28, and 42 of immunization.

TABLE 8

| Test Articles | Day 0 | Day 1 | Day 2 | Day 3 | Day 6 | Day 9 | Day 14 | Day 23 |
|---|---|---|---|---|---|---|---|---|
| PSII-CRM₁₉₇:End- | 0% | 0.02% | −12.44% | −10.35% | −5.90% | −2.26% | 0.16% | 3.82% |
| Linked (20 μg) | | | | | | | | |
| PSII-CRM₁₉₇:End- | 0% | −2.74% | −7.10% | −5.74% | −1.86% | −1.45% | 0.59% | 7.46% |
| Linked (45 μg) | | | | | | | | |
| PSII-CRM₁₉₇:End- | 0% | −2.57% | −8.73% | −8.78% | −7.89% | −2.62% | 1.37% | 4.47% |
| Linked(90 μg) | | | | | | | | |
| PSII-CRM₁₉₇:CDAP | 0% | −6.01% | −8.48% | −6.91% | −1.69% | −0.23% | 3.21% | 4.25% |
| (20 μg) | | | | | | | | |
| PSII-CRM₁₉₇:CDAP | 0% | 0.61% | −5.60% | −7.27% | −5.75% | −2.85% | −0.15% | 3.74% |
| (45 μg) | | | | | | | | |
| PSII-CRM₁₉₇:CDAP | 0% | 0.30% | −9.10% | −10.35% | −3.17% | 0.04% | −0.44% | 4.38% |
| (90 μg) | | | | | | | | |
| PSII-CRM₁₉₇:TEMPO | 0% | −0.11% | −3.89% | −7.06% | −6.25% | −0.24% | 0.15% | 3.08% |
| (20 μg) | | | | | | | | |
| PSII-CRM₁₉₇:TEMPO | 0% | −1.36% | −9.74% | −11.13% | −7.36% | −2.22% | −0.16% | 2.90% |
| (45 μg) | | | | | | | | |
| PSII-CRM₁₉₇:TEMPO | 0% | −1.73% | −13.46% | −10.65% | −9.88% | −5.75% | −2.07% | 1.03% |
| (90 μg) | | | | | | | | |
| Saline/Spores | 0% | −1.56% | −6.43% | −8.64% | −6.32% | 0.14% | 1.46% | 9.82% |
| Saline/No Spores | 0% | 0.44% | 0.41% | 0.11% | 2.73% | 2.42% | 2.88% | 8.22% |

TABLE 9

| Anti-PSII IgG ELISA Titers | | | | | | | | | |
|---|---|---|---|---|---|---|---|---|---|
| (TEMPO PSII Biotinylated | | | | | | | | | |
| ELISA) | | | Day 0 | Day 14 | | Day 28 | | Day 42 | |
| | | Dose | Pooled | GMT* | Sero- | GMT | Sero- | GMT | Sero- |
| Group | Description | (μg) | Titer | (n = 8) | conversion | (n = 8) | conversion | (n = 8) | conversion |
| 1 | PSII- | 20 | 25 | 25 | pool | 30 | 14%(1) | 28 | 0% |
| | CRM₁₉₇:RedAm | | | | | | | | |
| 2 | PSII- | 45 | 25 | 25 | pool | 25 | 0% | 25 | 0% |
| | CRM₁₉₇:RedAm | | | | | | | | |
| 3 | PSII- | 90 | 25 | 25 | pool | 27 | 0% | 25 | 0% |
| | CRM₁₉₇:RedAm | (45*2) | | | | | | | |
| 4 | PSII- | 20 | 25 | 25 | pool | 27 | 0% | 25 | 0% |
| | CRM₁₉₇:CDAP | | | | | | | | |
| 5 | PSII- | 45 | 25 | 25 | pool | 25 | 0% | 25 | 0% |
| | CRM₁₉₇:CDAP | | | | | | | | |
| 6 | PSII- | 90 | 25 | 25 | pool | 25 | 0% | 25 | 0% |
| | CRM₁₉₇:CDAP | (45*2) | | | | | | | |
| 7 | PSII- | 20 | 25 | 65 | 25% | 336 | 88% | 436 | 75% |
| | CRM₁₉₇:TEMPO | | | | | | | | |
| 8 | PSII- | 45 | 25 | 27 | 0% | 183 | 75% | 100 | 63% |
| | CRM₁₉₇:TEMPO | | | | | | | | |
| 9 | PSII- | 90 | 25 | 130 | 75% | 1,467 | 100% | 2,263 | 100% |
| | CRM₁₉₇:TEMPO | (45*2) | | | | | | | |
| 10 | Vehicle, Spored | N/A | 25 | 25 | pool | 25 | 0% | 25 | 0% |
| 11 | Vehicle, UnSpored | N/A | 25 | 25 | pool | 25 | 0% | 25 | 0% |

TABLE 10 shows immunogenicity of PSII-CRM₁₉₇ conjugates in mice using a biotinylated PSII ELISA-Day 42 pooled sera titers. The data show that

TABLE 10

| | | | Anti-PSII IgG ELISA Titers by ELISA Coating Antigen | | | |
|---|---|---|---|---|---|---|
| | Description of | Dose | Day42² | | | RedAm |
| Group | Test conjugates¹ | (ug) | End-linked | CDAP1 | CDAP2 | (mono PSII) |
| 1 | PSII-CRM₁₉₇ | 20 | 3,200 | 800 | 400 | 400 |
| 2* | Conjugate End- | 45 | 200 | 200 | 25 | 50 |
| 3 | linked RedAm | 90 | 400 | 200 | 25 | 200 |
| 4* | PSII-CRM₁₉₇ | 20 | 25 | >6400 | 800 | 25 |
| 5 | Conjugate CDAP | 45 | 25 | 400 | 800 | 25 |
| 6 | | 90 | 200 | 3,200 | 800 | 25 |
| 7 | PSII-CRM₁₉₇ | 20 | >6400 | 1,600 | 1,600 | 3,200 |
| 8 | Conjugate | 45 | 800 | 3,200 | 800 | 800 |
| 9 | TEMPO | 90 | 50 | 3,200 | 3,200 | 3,200 |

¹Conjugates were adjuvanted with 50 ug Alhydrogel per dose
²A total of 3 immunizations were administrated intramuscularly (IM) on Day 0, 14 and 28. Sera samples were collected 2 weeks post the last immunization (on Day 42) and assayed by PSII-specific ELISA. Titers from each group were showed above.
*indicates groups that showed in vivo protection against the spore challenge.

### Example 6: Dose Ranging Studies of PSII Conjugates

The effect of increased doses of PSII conjugates is investigated. Female 6-week-old C57/BL6 mice (n=105) are assigned to groups receiving increasing dose levels of RedAM, TEMPO, or CDAP PSII-CRM₁₉₇ conjugates, 3 times at a biweekly interval. The mice are then treated with an antibiotic treatment and challenged with 10⁴ CFU C. difficile spores. The mice are monitored for weight loss and symptoms of CDI. Blood samples are harvested for immunogenicity, and fecal pellets are harvested to test for colonization endpoints. The primary endpoints of the study are: 1) weight loss or symptoms following challenge on days 0, 1, 2, 3, 6, 9, and 14; 2) PSII IgG responses on study days 0, 14, 28, 42, and termination. The secondary endpoints of the study are fecal CFU on days 0, 6, and 14.

### Example 7: Synthesis and Characterization of End-Linked PSII-CRM₁₉₇ Conjugates

The general scheme for synthesizing the End-linked PSII-CRM₁₉₇ conjugate is shown in SCHEME 4.

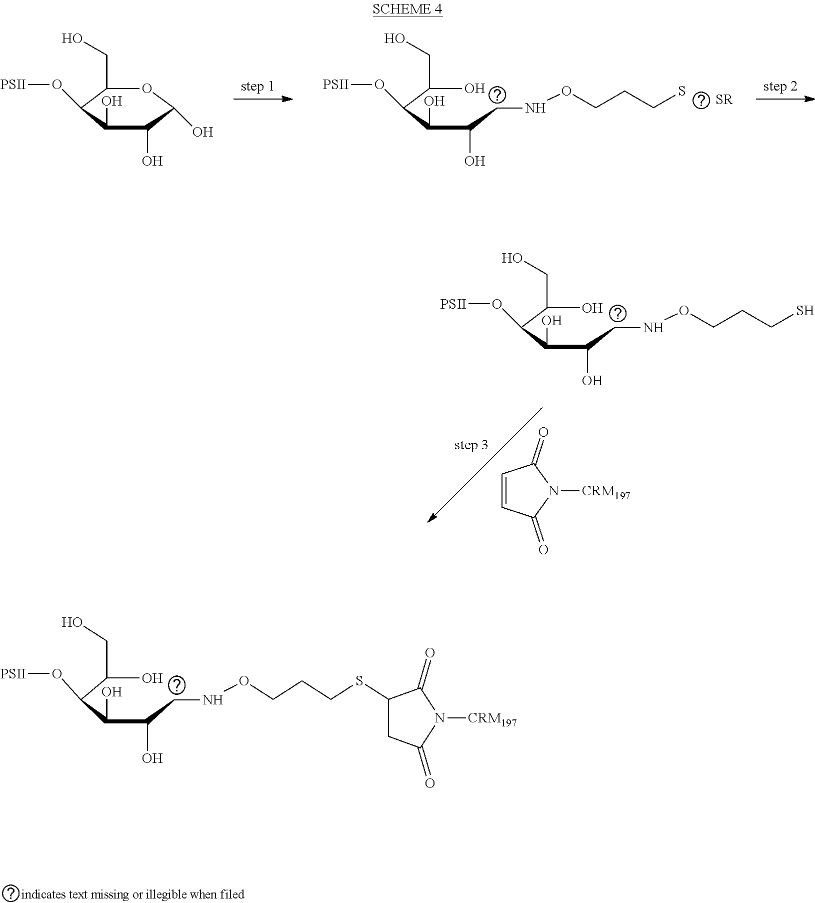

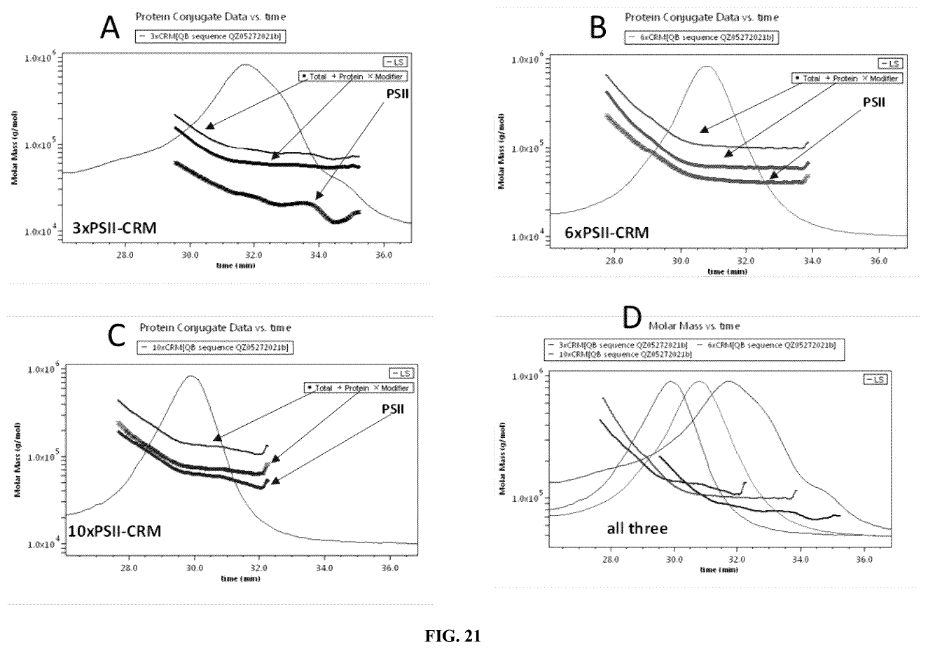

The conjugate were separated from unconjugated PRI and CRM₁₉₇ using size exclusion chromatography, and the molecular weight and degree of conjugation were estimated using multi-angle laser light scattering (SEC-MALS). A representative analysis of an End-linked PSII-CRM₁₉₇ conjugate by SEC-MALS is shown in FIG. 5. The molecular weight of the conjugate was 111.8 kDa, which corresponded to a single CRM₁₉₇-maleimide molecule (61.6 kDa) containing approximately 6 chains of PSII (10 kDa/chain). The degree of conjugation, however, could be controlled by altering the ratio of derivatized PSII to CRM₁₉₇-maleimide. End-linked PSII-CRM₁₉₇ conjugates containing an average of 3, 6, and 10 PSII chains per CRM₁₉₇ were prepared to determine the optimal PSII to CRM₁₉₇ ratio with regard to immunogenicity and protection in murine models of CDI. The SEC-MALS chromatogram data from the analysis of the 3 End-linked PSII-CRM₁₉₇ conjugates are shown in FIG. 21. The estimated molecular weight and degree of conjugation are shown in TABLE 12 below.

Mono-functionalization of PSII (Step 1): 75 mg 3-aminooxypropane-1-thiol HCl salt (40 mole equivalents to PSII) was first dissolved in 4 mL 1.0 M borate buffer (pH adjusted to 8.5) in a glass vial. Then, 5 mL CPS20 PSII (19.8 mg/mL solution in water) was mixed with the 3-aminooxypropane-1-thiol solution. The resulting solution was incubated at 50° C. for 18 h and then cooled down to room temperature. The pH of the reaction solution was measured as 8.79. The stability of the PSII was confirmed by SEC-MALS analysis.

Reduction of the PSII oxime was carried out at room temperature. The pH of the reaction solution was first adjusted to 8.5. Sodium borohydride solid was added in 3 successive batches (50 mg each, 150 mg total). The pH of the reaction solution was maintained between 8.4-8.6 during the reduction process by adding 1 N HCl solution over 60 min. The reaction was quenched by adjusting the pH to 6.8 with a 1 N HCl solution. The resulting solution was passed through a 0.2 μm filter (Whatman GD/X PES) to remove insoluble particles, and a final volume of 15 mL was obtained. SEC MALS analysis confirmed the integrity of PSII and determined the concentration to be 6 mg/mL.

The level of the thiol content of the resulting PSII-NH₂ORSSR was assessed using a 5,5-dithio-bis-(2-nitrobenzoic acid) (DTNB) assay. A 50 μL, aliquot of PSII-NH₂ORSSR was mixed with 100 μL of a tris(2-carboxyethyl)phosphine (TCEP) solution (6 mg TCEP HCl salt in 100 mM sodium acetate buffer, final pH 6.0). The reaction proceeded at room temperature for 1 h. Then TCEP, excess aminooxythiol, and other small molecules were removed by ultra-centrifugation with a 3 K Amicon ultrafilter (4,000 rpm×25 min) using 5 mM EDTA in 5 mM sodium acetate buffer (pH 6.5) as the washing buffer. Four rounds of centrifugation were carried out. The filtrate after 4^{th} centrifugation had an OD below the limit of detection at 410 nm. The thiol concentration was calculated as 0.087 mM, resulting in a yield of 77% incorporation of aminooxyl thiol per PSII molecule (7.6 KDa) at the reducing end. The stability of PSII was confirmed by SEC-MALS analysis.

Conjugation of PSII to CRM₁₉₇-Maleimide (Steps 2 & 3): Conjugation of modified PSII to CRM₁₉₇-maleimide was carried out at the 3×, 6× or 10× mole ratio of PSII (7.6 K) to CRM-maleimide. TCEP (6 mg/mL) in 100 mM sodium phosphate buffer (pH 6.0, 8 mL) was added to a solution of PSII-NH₂ORSSR (6 mg/mL, 4 mL) at room temperature. After 1 h, TCEP, excess aminooxythiol and other small molecules were removed by ultracentrifugation with 3 K Amicon ultrafilter (4,000 rpm×25 min) using 5 mM EDTA in 5 mM sodium phosphate buffer (pH 6.6) as the washing buffer. Five rounds of centrifugation were carried out. PSII free thiol as the retentate was determined by DTNB assay. The resulting PSII free thiol solution was then mixed with CRM₁₉₇-maleimide (5 mg/mL in PBS and 5 mM EDTA, pH 6.8) at different ratios (TABLE 11). The conjugation reaction was facilitated by adding 3 mL conjugation buffer (0.5 M PBS, 10 mM EDTA, pH 7.5). The reaction solutions were left at room temperature for 2 h and then at 4° C. for 16 h. The reactions were quenched by adding a mercaptoethanol solution (0.5 M in water, 10 μL). TABLE 11 shows the amount of PSII free thiol used to CRM₁₉₇ Maleimide at 3×, 6×, and 9× ratios in conjugation.

TABLE 11

| | | |
|---|---|---|
| Ratio | CRM₁₉₇-maleimide | PSII-NHORSH |
| (PSII to CRM) | (5 mg/mL) | (0.3 mM) |

| | | | | |
|---|---|---|---|---|
| 3× | 5 mg | (86 nmol) | 250 nmol | 0.83 mL |
| 6× | 5 mg | (86 nmol) | 520 nmol | 1.73 mL |
| 10× | 2.5 mg | (43 nmol) | 430 nmol | 1.43 mL |

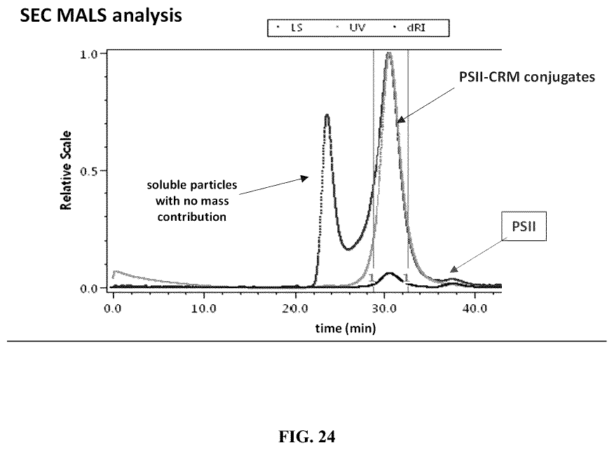

The conjugation of PSII to CRM₁₉₇ maleimide in all three reactions was confirmed by SEC MALS analysis. FIG. 24 shows a representative analysis FIG. 6×PSII-CRM), which had a PSII-conjugate signal at 30 min, and PSII and PSII thiol peaks around 38 min. There was an unknown peak at 24 min with no detectable mass nor UV absorbance, which may have been soluble particles present in all three reactions. The resulting conjugation reaction solutions was diluted with PBS to 12 mL. PSII and other small molecules in solutions in the reaction solution were removed by centrifugation with 30 K Amicon ultrafilter (4000 RPM×15 min), which afforded a final concentrated solution with a volume of approximately 300 μL. No significant mass loss was found by SEC MALS analysis after the centrifugation. The obtained final PSII-CRM₁₉₇ conjugates (2.5-3.0 mL each) was sterilized by passing a sterile 0.22 filter and fully analyzed by SEC MALS analysis.

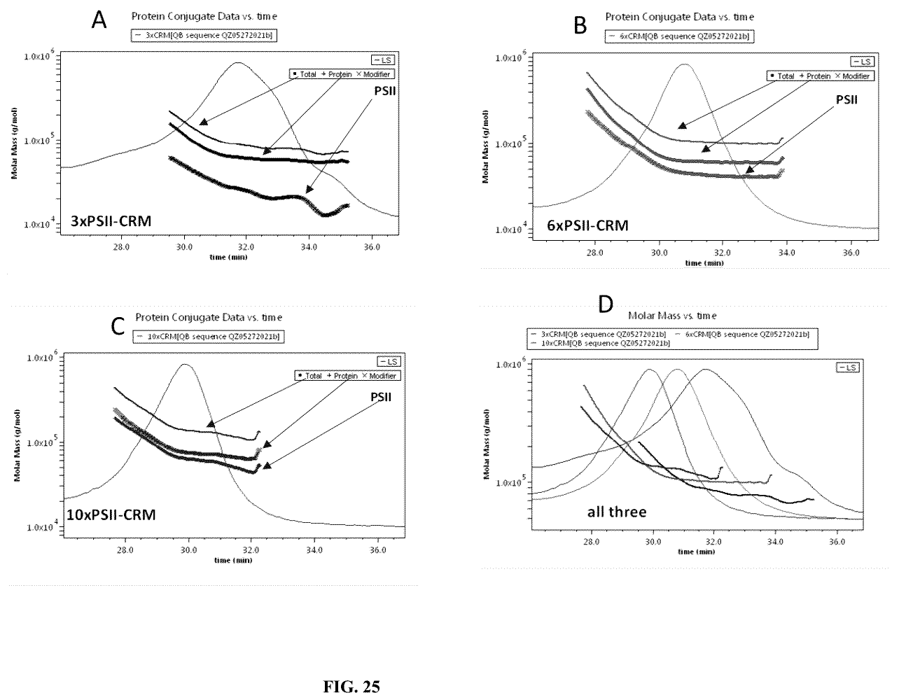

FIG. 25 shows the final SEC MALS analysis results with detailed components of CRM₁₉₇ protein and PSII across the conjugate signals and an analysis with the components combined. TABLE 12 shows the detailed mass units and the determination of PSII conjugation ratios.

TABLE 12

| | | | | CRM₁₉₇ | PSII | | | | |
|---|---|---|---|---|---|---|---|---|---|
| | | | | part | part | CRM₁₉₇ | PSII | mole | |
| | Mn | Mw | polydispersity | Mw | Mw | mass | mass | ratio | |
| | (kDa) | (kDa) | (Mw/Mn) | (kDa) | (kDa) | (μg) | (μg) | (PSII:CRM) | |
| 3x | 86.3 | 90.9 | 1.054 | 65.4 | 25.5 | 59.39 | 22.6 | 3.4 | (3.2) |
| 6x | 114.0 | 124.2 | 1.089 | 72.6 | 51.5 | 53.51 | 38.25 | 6.8 | (6.0) |
| 10x | 145.8 | 156.4 | 1.073 | 71.3 | 85.2 | 22.08 | 26.57 | 11.2 | (10.1) |

Two methods were used to calculate the ratio of PSII to CRM₁₉₇ in the conjugate. The first method used the formula (MW_{conj}-MW_{CRM})/7.6 kDa. The second values (listed in parenthesis) used the MALS determined CRM₁₉₇ and PSII mass of the conjugate with the formula: (PSII mass/CRM₁₉₇ mass)*62 kDa/7.6 kDa (CRM₁₉₇ maleimide as 62 kDa). The results from both calculations indicated consistent targeted ratios of PSII to CRM₁₉₇ in the conjugates. The final concentration, volume and amounts of sterile PSII-CRM₁₉₇ conjugate solutions were: 1) 3×PSII-CRM₁₉₇ conjugate 3.0 mg/mL in PBS, 2.2 mL (6.6 mg); 2) 6×PSII-CRM₁₉₇ conjugate 3.4 mg/mL in PBS, 2.2 mL (7.6 mg); and 3) 10×PSII-CRM₁₉₇ conjugate 1.8 mg/mL in PBS, 2.7 mL (4.9 mg).

### Example 8: Effects of Adjuvants for C. Difficile Vaccine

Endpoints were anti-toxoid A, anti-toxoid B, Anti-PSII IgG responses as determined by ELISA. Anti-toxin A and anti-toxin B neutralizing antibody responses; weight loss and symptoms following CDI; and fecal CFU were measured.

Six groups of mice received either 45 μg of the PSII conjugate; 5 μg of toxoid A and 5 μg of toxoid B; 45 μg of the PSII conjugate, 5 μg of toxoid A, and 5 μg of toxoid B; or sterile saline. The experimental groups also received different adjuvants (AlPO₄, AlOH, or Advax2). Group 5, which received Advax2 as the adjuvant received two 100 μL injections.

TABLE 13 shows the study groups, dose administration, test articles, adjuvant, and volume injected for the experiment.

TABLE 13

| | Dose Admin. | Test Articles | Adjuvant | Volume | |
|---|---|---|---|---|---|
| Group No. | IM (μg) | Admin. IM | (1 mg/ml) | Injected (μL) | No. of Mice |
| 1 | 45 | PSII-CRM₁₉₇ | AlPO₄ | 50 | 10 |
| | | End-linked | | | |
| | | Conjugate | | | |
| 2 | 5 + 5 | Toxoids A + B | AlOH | 50 | 10 |
| 3 | (45) and | PSII-CRM₁₉₇ | AlPO₄ | 50 | 10 |
| | (5 + 5) | End-linked | | | |
| | | Conjugate and | | | |
| | | Toxoids A + B | | | |
| 4 | (45) and | PSII-CRM₁₉₇ | AlOH | 50 | 10 |
| | (5 + 5) | End-linked | | | |
| | | Conjugate and | | | |
| | | Toxoids A + B | | | |
| 5 | (45) and | PSII-CRM₁₉₇ | Advax2 | 100 (50 × 2) | 10 |
| | (5 + 5) | End-linked | | | |
| | | Conjugate and | | | |
| | | Toxoids A + B | | | |
| 6 | N/A | Sterile Saline | AlPO₄ | 50 | 9 |

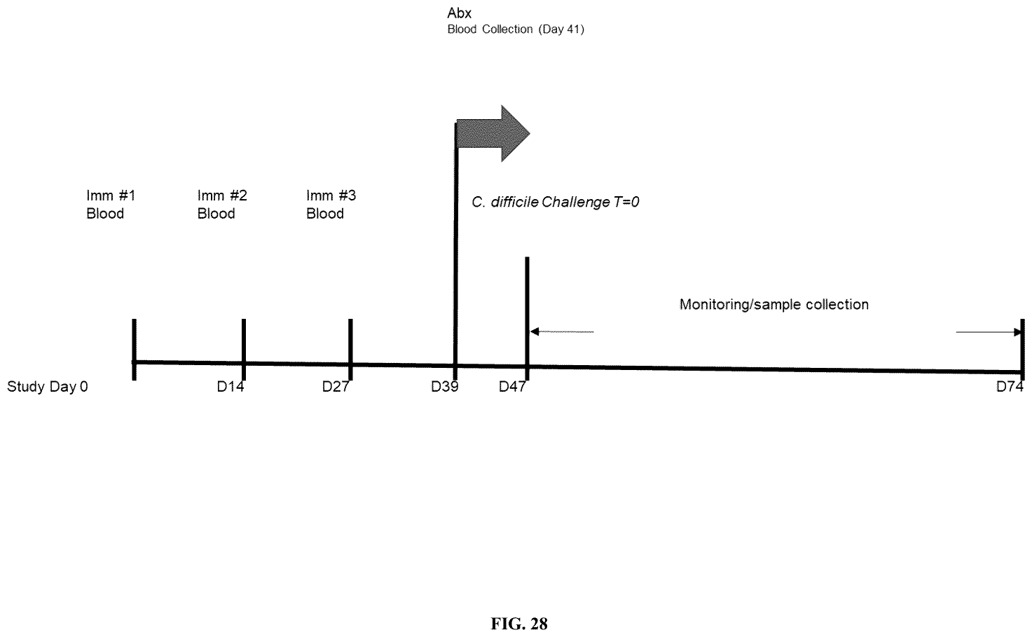

Timeline for the study. Three biweekly immunizations were administered on day 0, 14 and 27. Bleeds for ELISAs were performed on day 0, 14, 27, and 41 (2 weeks after final immunization). Around two weeks following the third immunization, an antibiotic regimen was administered followed by a spore challenge. The animals were monitored for about weeks, which included fecal collection and analysis. FIG. 28 illustrates the timeline for the study used to determine the effects of different adjuvants.

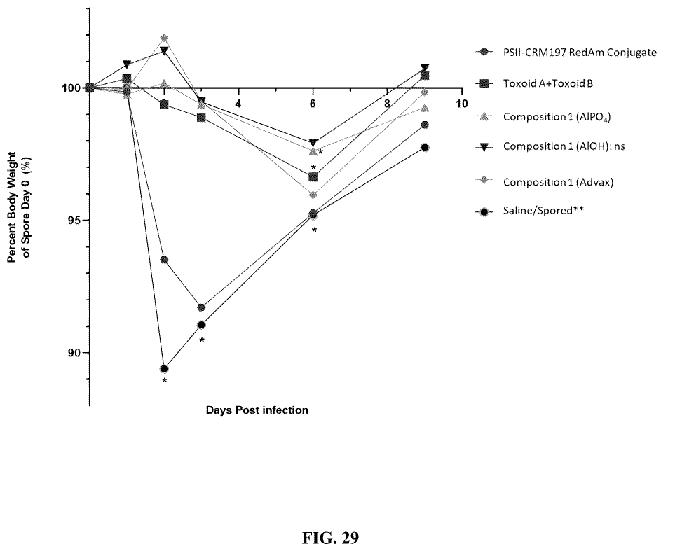

Composition 1 comprises 45 μg of the PSII-CRM₁₉₇ End-linked conjugate, 5 μg of toxoid A, and 5 μg of toxoid B. FIG. 29 shows the CDI geometric mean weight loss post-spore infection over the first 9 days after infection. Compared to saline-treated animals the treatment candidates exhibited less weight loss. For most days, mice treated with AlOH exhibited percent body weights compared to spore day 0 greater than mice treated with the toxoid combination alone. The animals treated with the vaccine composition did not exhibit significant weight loss compared to the saline/spored group, especially on days 2-6. On days 2-6, the saline/spored groups exhibited significant weight loss. The (*) for the saline/spored group in FIG. 29 indicates that there was statistically significant weight loss at days 2-6 post-infection compared to day 0. The saline/spored group (**) had two dead mice on day 3 post-infection. The (*) for the Toxoid A/B curve indicates that there was statistically significant weight loss at day 6 post-infection compared to day 0. The group treated with vaccine composition 1 (AlOH) did not exhibit statistically significant weight loss at any point post-infection. Statistical analysis was conducted by Two-way ANOVA (Dunnett's multiple comparison's test).

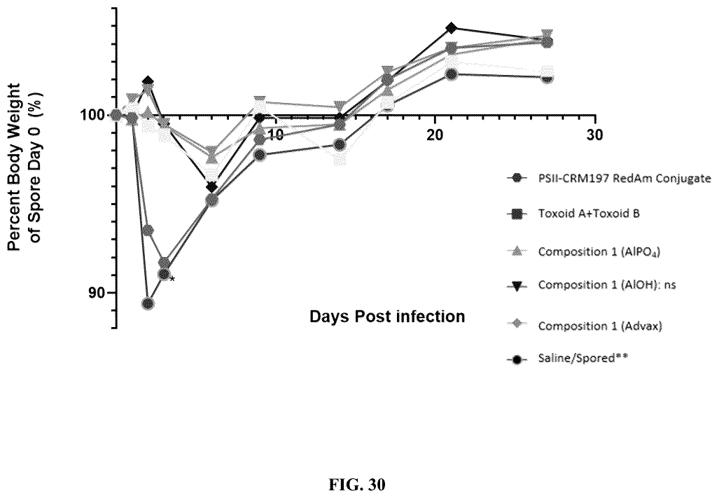

FIG. 30 shows the graph from FIG. 29 from day 0 to day 30. TABLE 14 shows the % weight changes upon immunization with vaccine composition 1, as detailed in TABLE 13. All treatment groups with toxoids A and B did not exhibit significant weight loss, and treatment groups that received composition 1 exhibited the most stability in body weight. Groups treated with Advax or ALPO₄ adjuvanted vaccine exhibited soft stools shortly after infection, and up to 10 days post-infection. The group treated with AlOH was symptom-free throughout the duration of the study and was the first group to return to normal body weight.

TABLE 14

| Test articles | D 1 | D 2 | D 3 | D 6 | D 9 | D 14 | D 17 | D 21 | D 27 |
|---|---|---|---|---|---|---|---|---|---|
| PSII-CRM₁₉₇ | −0.16% | −6.49% | −8.29% | −4.73% | −1.39% | −0.53% | 1.99% | 3.77% | 4.08% |
| End-linked | | | | | | | | | |
| conjugate | | | | | | | | | |
| Toxoids A + B | 0.35% | −0.63% | −1.11% | −3.36% | 0.47% | −2.5% | 0.7% | 3.01% | 2.51% |
| Composition | −0.24% | 0.18% | −0.62% | −2.37% | −0.73% | −0.53% | 1.41% | 3.41% | 4.27% |
| 1 (AlPO₄) | | | | | | | | | |
| Composition | 0.87% | 1.38% | −0.52% | −2.09% | 0.73% | 0.44% | 2.42% | 3.77% | 4.47% |
| 1 (AlOH) | | | | | | | | | |
| Composition | −0.05% | 1.89% | −0.5% | −4.04% | −0.17% | −0.16% | 1.91% | 4.89% | 4.22% |
| 1 (Advax) | | | | | | | | | |
| Saline/Spores | 0.01% | −10.61% | −8.97% | −4.8% | −2.24% | −1.67% | 0.54% | 2.3% | 2.12% |

Final titer tables were obtained for groups treated with the compositions and different adjuvants. Based on titer data alone, the group treated with composition 1 and AlPO₄ exhibited a superior immune response compared to other treatment with the other adjuvants or the toxoids alone. All samples were seroconverted by immunization Day 14, two weeks after first immunization. TABLE 15 and TABLE 16 show TxdA IgG ELISA Titer Summary data for treatment groups and the individual mice within treatment groups that were treated with the PSII-CRM₁₉₇ End-linked conjugate alone, toxoids A and B, or the conjugate, toxoids A and B, and AlPO₄, AlOH, or advax as an adjuvant.

TABLE 15

| | Dose | | Adjuvant/ | Anti-TxdA IgG GMT | | | |
|---|---|---|---|---|---|---|---|
| Group | (μg) | Test Article | dilution | Day 0 | Day 14 | Day 27 | Day 41 |
| 1 | 45 | PSII-CRM₁₉₇ End-linked | AlPO₄ | 25 | 25 | 25 | 25 |
| | | Conjugate | | | | | |
| 2 | 5 + 5 | Toxoids A + B | AlOH | 25 | 71,385 | 597,141 | 1,600,000 |
| 3 | (45) | PSII-CRM₁₉₇ End-linked | AlPO₄ | 25 | 129,960 | 1,114,305 | 2,985,706 |
| | and | Conjugate and Toxoids A + B | | | | | |
| | 5 + 5 | | | | | | |
| 4 | (45) | PSII-RM₁₉₇ End-linked | AlOH | 25 | 105,561 | 640,000 | 1,600,000 |
| | and | Conjugate and Toxoids A + B | | | | | |
| | 5 + 5 | | | | | | |
| 5 | (45) | PSII-CRM₁₉₇ End-linked | Advax2 | 25 | 74,643 | 1,600,000 | 1,837,917 |
| | and | Conjugate and Toxoids A + B | | | | | |
| | 5 + 5 | | | | | | |
| 6 | N/A | Sterile Saline | AlPO₄ | 25 | 25 | 29 | 25 |

TABLE 16

| | Description | | Adjuvant | | Sero- | | | | |
|---|---|---|---|---|---|---|---|---|---|
| | of | Dose | (1 mg/ | GMT | conversion | Anti-TxdA IgG GMT for Individual Mice | | | |
| Group | group | (ug) | ml) | (n = 10) | (%) | 1 | 2 | 3 | 4 |
| 1 | PSII- | 45 | AlPO₄ | 25 | 0 | | | | |
| | CRM₁₉₇:End- | | | | | | | | |
| | linked | | | | | | | | |
| 2 | Toxoids | 5+ | AlOH | 71,385 | 100 | 160,000 | 80,000 | 80,000 | 32,000 |
| | A + B | | | | | | | | |
| 3 | PSII- | 45 | AlPO₄ | 129,960 | 100 | 80,000 | 40,000 | 160,000 | 160,000 |
| | CRM₁₉₇:End- | and | | | | | | | |
| | linked | (5 + 5) | | | | | | | |
| | and | | | | | | | | |
| | Toxoids | | | | | | | | |
| | A + B | | | | | | | | |
| 4 | PSII- | 45 | AlOH | 105,561 | 100 | 160,000 | 160,000 | 80,000 | 80,000 |
| | CRM₁₉₇:End- | and | | | | | | | |
| | linked | (5 + 5) | | | | | | | |
| | and | | | | | | | | |
| | Toxoids | | | | | | | | |
| | A + B | | | | | | | | |
| 5 | PSII- | 45 and | Advax2 | 74,643 | 100 | 40,000 | 80,000 | 80,000 | 80,000 |
| | CRM₁₉₇:End- | (5 + 5) | | | | | | | |
| | linked | | | | | | | | |
| | and | | | | | | | | |
| | Toxoids | | | | | | | | |
| | A + B | | | | | | | | |
| 6 | Sterile | N/A | AlPO₄ | 25 | 0 | 25 | 25 | 25 | 25 |
| | Saline | | | | | | | | |

| | Anti-TxdA IgG GMT for Individual Mice | | | | | |
|---|---|---|---|---|---|---|
| Group | 5 | 6 | 7 | 8 | 9 | 10 |
| 1 | | | | | | |
| 2 | 32,000 | 40,000 | 80,000 | 80,000 | 160,000 | 80,000 |
| 3 | 160,000 | 80,000 | 320,000 | 160,000 | 160,000 | 160,000 |
| 4 | 80,000 | 160,000 | 80,000 | 80,000 | 80,000 | 160,000 |
| 5 | 80,000 | 80,000 | 80,000 | 80,000 | 80,000 | 80,000 |
| 6 | 25 | 25 | 25 | 25 | 25 | 25 |

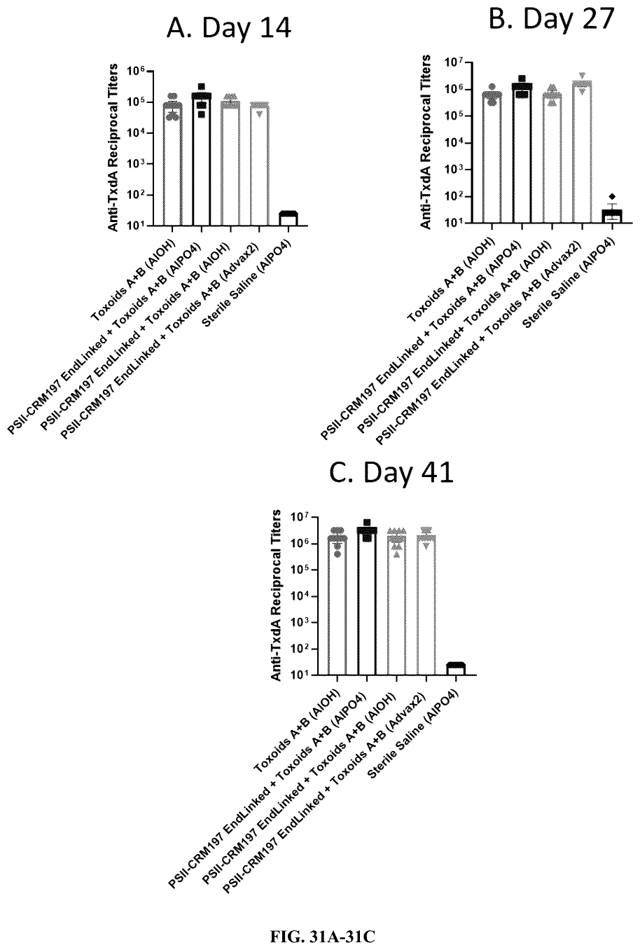

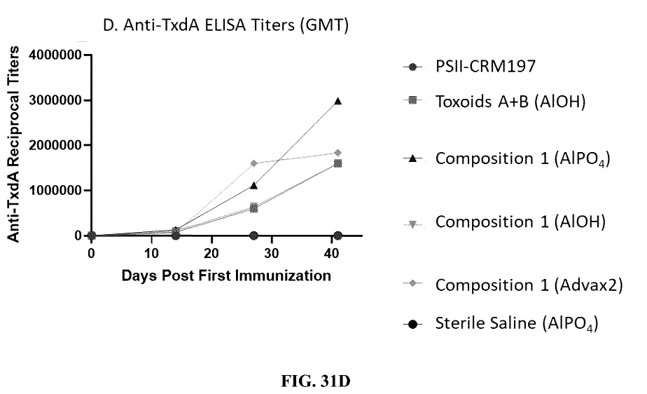

FIG. 31A-C show anti-TcdA reciprocal titers in groups treated with toxoids A and B (AlOH); PSII-CRM₁₉₇ End-linked+toxoids A and B+AlPO₄; PSII-CRM₁₉₇ End-linked+toxoids A and B+A10H; PSII-CRM₁₉₇ End-linked+toxoids A and B+Advax2; and sterile saline (AlPO₄) on day 14 (FIG. 31A), day 27 (FIG. 31B), and day 41 (FIG. 31C). FIG. 31D shows anti-TxdA ELISA Titers for groups treated with PSII-CRM₁₉₇; toxoids A and B (AlOH); PSII-CRM₁₉₇ End-linked+toxoids A and B+AlPO₄; PSII-CRM₁₉₇ End-linked+toxoids A and B+A10H; PSII-CRM₁₉₇ End-linked+toxoids A and B+Advax2; and sterile saline (AlPO₄) throughout the duration of the study.

TABLE 17 and TABLE 18 show TxdB IgG ELISA Titer Summary data for treatment groups and the individual mice within treatment groups that were treated with the PSII-CRM₁₉₇ End-linked conjugate alone, toxoids A and B, or the conjugate, toxoids A and B, and AlPO₄, AlOH, or Advax as an adjuvant.

TABLE 17

| | Dose | | Adjuvant/ | | | | |
|---|---|---|---|---|---|---|---|
| Group | (μg) | Test Article | dilution | Day 0 | Day 14 | Day 27 | Day 41 |
| 1 | 45 | PSII-CRM₁₉₇ End-linked | AlPO₄ | 25 | 25 | 25 | 50 |
| | | Conjugate | | | | | |
| 2 | 5 + 5 | Toxoids A + B | AlOH | 25 | 149,285 | 844,485 | 1,371,870 |
| 3 | (45) | PSII-CRM₁₉₇ End-linked | AlPO₄ | 25 | 89,868 | 452,548 | 905,097 |
| | and | Conjugate and Toxoids A + B | | | | | |
| | 5 + 5 | | | | | | |
| 4 | (45) | PSII-RM₁₉₇ End-linked | AlOH | 25 | 296,983 | 1,114,305 | 1,688,970 |
| | and | Conjugate and Toxoids A + B | | | | | |
| | 5 + 5 | | | | | | |
| 5 | (45) | PSII-CRM₁₉₇ End-linked | Advax2 | 25 | 8,574 | 685,935 | 1,810,193 |
| | and | Conjugate and Toxoids A + B | | | | | |
| | 5 + 5 | | | | | | |
| 6 | N/A | Sterile Saline | AlPO₄ | 25 | 33 | 27 | 31 |

TABLE 18

| | | | | | Sero- | | | | |
|---|---|---|---|---|---|---|---|---|---|
| | | Dose | Adjuvant | GMT | conversion | Mouse | | | |
| Group | Description | (ug) | (1 mg/ml) | (n = 10) | (%) | 1 | 2 | 3 | 4 |
| 1 | PSII- | 45 | AlPO₄ | 25 | 0 | | | | |
| | CRM₁₉₇:End- | | | | | | | | |
| | linked | | | | | | | | |
| 2 | Toxoids | 5 + 5 | AlOH | 149,285 | 100 | 160,000 | 160,000 | 160,000 | 160,000 |
| | A + B | | | | | | | | |
| 3 | PSII- | 45 | AlPO₄ | 89,868 | 100 | 160,000 | 80,000 | 320,000 | 80,000 |
| | CRM₁₉₇:End- | and | | | | | | | |
| | linked | (5 + 5) | | | | | | | |
| | and | | | | | | | | |
| | Toxoids | | | | | | | | |
| | A + B | | | | | | | | |
| 4 | PSII- | 45 | AlOH | 196,983 | 100 | 320,000 | 160,000 | 160,000 | 160,000 |
| | CRM₁₉₇:End- | and | | | | | | | |
| | linked | (5 + 5) | | | | | | | |
| | and | | | | | | | | |
| | Toxoids | | | | | | | | |
| | A + B | | | | | | | | |
| 5 | PSII- | 45 and | Advax2 | 8,574 | 100 | 32,000 | 8,000 | 8,000 | 8,000 |
| | CRM₁₉₇:End- | (5 + 5) | | | | | | | |
| | linked | | | | | | | | |
| | and | | | | | | | | |
| | Toxoids | | | | | | | | |
| | A + B | | | | | | | | |
| 6 | Sterile | N/A | AlPO₄ | 33 | 0 | 25 | 25 | 25 | 25 |
| | Saline | | | | | | | | |

| | Mouse | | | | | |
|---|---|---|---|---|---|---|
| Group | 5 | 6 | 7 | 8 | 9 | 10 |
| 1 | | | | | | |
| 2 | 320,000 | 80,000 | 160,000 | 160,000 | 160,000 | 80,000 |
| 3 | 80,000 | 32,000 | 40,000 | 80,000 | 160,000 | 80,000 |
| 4 | 160,000 | 160,000 | 160,000 | 320,000 | 160,000 | 320,000 |
| 5 | 8,000 | 4,000 | 8,000 | 16,000 | 8,000 | 4,000 |
| 6 | 25 | 100 | 25 | 100 | 25 | 25 |

### Example 9: End-Linked PSII-CRM₁₉₇ Conjugate Immunogenicity and Protection

PSII was first functionalized with a thiol group at the reducing end through oxime formation followed by reductive amination. The conjugation to CRM₁₉₇ was then achieved through the free thiol addition to the CRM₁₉₇ maleimide. The final 6×PSII-EcoCRM conjugate solution was sterilized, and the final product was characterized by SEC-MALS analysis.

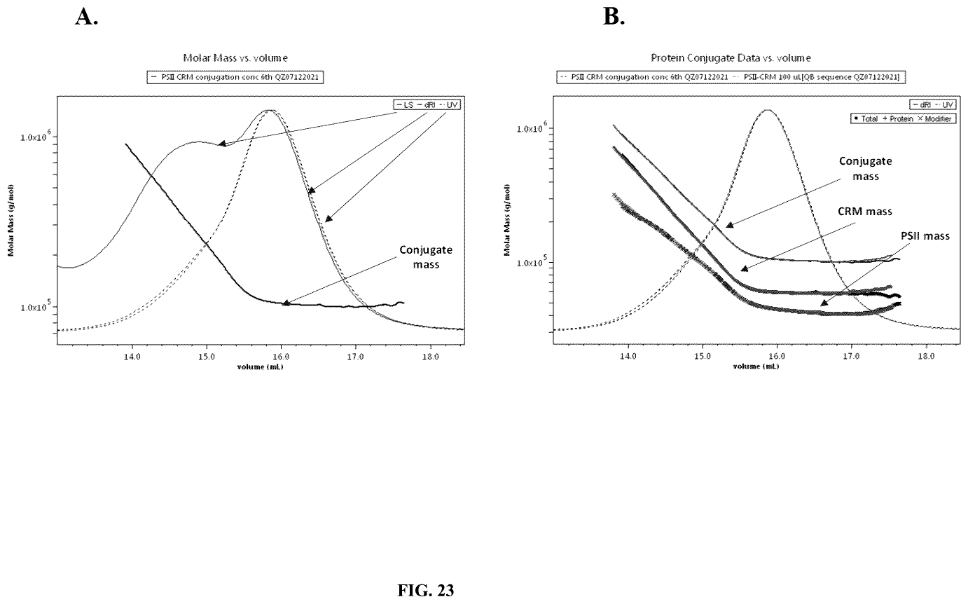

For the analysis of conjugate, 50 μL of the final 6×PSII-EcoCRM conjugate solution was diluted with 250 μL PBS solution. The UV absorbance at 280 nm of the diluted sample was recorded to establish the CRM₁₉₇ concentration. Samples in duplicate (50 and 100 μL injection) were analyzed by SEC-MALS with TOSOH TSKgel G4000PW column followed by TSKgel G2000PW_{xL} and PBS containing 0.02% sodium azide as the running buffer at a flow rate of 0.5 mL/min. For conjugate analysis, dn/dc for PSII was 0.137, UV co-eff was 0; dn/dc for CRM₁₉₇ was 0.185 and UV coeff was 0.934. The determined masses, concentrations, and ratios in the PSII-CRM₁₉₇ conjugates are summarized in TABLE 19. The ratio of PSII to CRM₁₉₇ in the conjugate was calculating using the following formula: (PSII mass/CRM₁₉₇ mass)*62 kDa/7.6 kDa (CRM₁₉₇ maleimide as 62 kDa). Representative data can be found in FIG. 23.

TABLE 19

| | CRM | | | CRM | PSII | | | |
|---|---|---|---|---|---|---|---|---|
| | OD₂₈₀ | Final | | part | part | CRM | PSII | |
| | 1:6 | CRM | polydispersity | Mw | Mw | mass | mass | mole ratio |
| | dilution | (mg/mL) | (Mw/Mn) | (kDa) | (kDa) | (μg) | (μg) | (PSII:CRM) |
| Tube | 0.625 | | 1.252 | 92 | 64 | 67.8 | 49.6 | 5.97^{(mass)} |
| #A | | | | | | | | |
| | (0.67 mg/mL) | 4.02 | | | Final PSII conc = 3.0 mg/mL | | | (49.6/67.8)*62/7.6 |
| Tube | 0.570 | | 1.272 | 95 | 55 | 63.4 | 46.2 | 5.95^{(mass)} |
| #B | | | | | | | | |

| | | | |
|---|---|---|---|
| (0.61 mg/mL) | 3.66 | Final PSII conc = 2.8 mg/mL | (46.2/63.4)*62/7.6 |

| | |
|---|---|
| Total EcoCRM = 90 mg | Total PSII = 34.5 + 32.2 = 66.7 mg |

70 mg of the end-linked PSII-CRM₁₉₇ containing an average of 6 chains was produced for further evaluation in murine models of CDI. Several murine studies were conducted to examine the impact of dose levels of the End-Linked PSII-CRM₁₉₇ conjugate on immunogenicity in 2 murine models of CDI. The first murine model evaluated the vaccine in prevention of disease symptoms of primary CDI as measured through weight loss. The second model was a secondary prophylaxis (colonization) model where the mice were first infected with C. difficile and then vaccinated to determine impact of the vaccination on C. difficile CFU in feces.

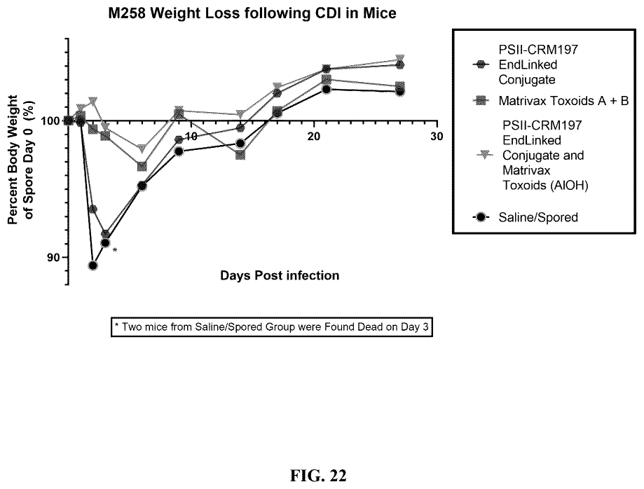

Naïve mice (N=5-8) were immunized 3 times biweekly with 45 μg PSII-CRM₁₉₇ End-linked conjugate with or without 5 μg of Toxoid A and B each. Two weeks after the last immunization, mice were inoculated with 10⁷ C. difficile spores IG. The body weight of each animal was monitored up to daily for 27 days. The geometric mean of percentage weight loss of each immunized group were graphed. Two mice from the saline immunized group were found dead on Day 3 post spore challenge. There were no other deaths in this experiment. The results from the murine primary CDI model showed little-to-no impact of the PSII-CRM₁₉₇ conjugate on the initial weight loss following infection, which is mediated primarily through the action of the 2 major toxins produced by C. difficile. There was some evidence for an improved rate of weight recovery in the vaccinated mice (FIG. 22). FIG. 22 shows protection against CDI following immunizations of PSII-CRM₁₉₇ conjugate with and without toxoids A and B. (*) indicates two mice from the saline immunized group were found dead on Day 3 post spore challenge.

The immunogenicity of the End-linked PSII-CRM₁₉₇ conjugates with different ratios of PSII chains to CRM₁₉₇ was evaluated in mice. In mice, the conjugate with an average of 6 PSII chains elicited the highest anti-PSII IgG titer. All conjugates exhibited some immunogenicity.

TABLE 20 shows the conjugate ratio study design. Primary endpoints were: 1) serum PSII IgG on Day 0, 14, 28, and 42 (2 weeks post each immunization); and 2) weight loss following challenge.

TABLE 20

| | | | Volume | | |
|---|---|---|---|---|---|
| | Test articles | Adjuvant | injected | No. | |
| Group | administered IM | (1 mg/mL) | (μL) | mice | Infection |
| 1 | 3:1 PSII: CRM₁₉₇ | AlOH | 50 | 8 | Y (Spores) |
| 2 | 6:1 PSII: CRM₁₉₇ | AlOH | 50 | 8 | Y (Spores) |
| 3 | 6:1 PSII: CRM₁₉₇ | AlOH | 50 | 8 | Y |
| | | | | | (Veg cells) |
| 4 | 10:1 PSII: CRM₁₉₇ | AlOH | 50 | 8 | Y (Spores) |
| 5 | Saline | AlOH | 50 | 8 | Y (Spores) |
| 6 | Saline | AlOH | 50 | 8 | Y |
| | | | | | (Veg cells) |
| 7 | Saline | AlOH | 50 | 5 | N |

TABLE 21 shows the immunogenicity of the end-linked PSII-CRM₁₉₇ conjugates with different PSII:CRM₁₉₇ ratios. A 6:1 ratio exhibited the most desirable effects

TABLE 21

| | | | Anti-PSII IgG GMT | | | |
|---|---|---|---|---|---|---|
| Groups | Description of conjugates¹ | | Pre-immune | D 14 | D 28 | D 42 |
| 1 | 3:1 PSII:CRM₁₉₇ Conjugate (45 μg) | Pooled | 25 | 100 | 4,800 | 9,600 |
| | | GMT | ND² | ND² | 6,400 | 12,070 |
| 2 | 6:1 PSII:CRM₁₉₇ Conjugate (45 μg) | Pooled | 25 | 800 | 19,200 | 32,000 |
| | | GMT | ND² | 39 | 10,475 | 55,834 |
| 3 | 10:1 PSII:CRM₁₉₇ Conjugate (45 μg) | Pooled | 25 | 800 | 19,200 | 32,000 |
| | | GMT | ND² | 1,467 | 8,880 | 35,338 |
| 4 | Sterile Saline | Pooled | 25 | 25 | 25 | 25 |

¹Conjugates were adjuvanted with 50 μg Alhydrogel (AlOH) per dose. A total of 3 immunizations were administrated IM on Day 0, 14 and 28.
²ND indicates the timepoints samples were only assayed as a pooled group sample, not individually.

Immunogenicity data indicated that infection of C. difficile prior to first immunization of PSII conjugate vaccine elicited faster and more robust serum anti-PSII IgG titers in mice (TABLE 10). Although initial measurements of anti-PSII IgG levels by ELISA were inconsistent with regard level and correlation to protection data, changes in the coating antigen yielded more consistent titers that correlate better with protection data. TABLE 22 shows the immunogenicity of different PSII:CRM₁₉₇ ratios following immunizations in naïve mice or C. difficile infection-primed mice.

TABLE 22

| | | | Adjuvant | | Days post 1^{st} immunization | | | | |
|---|---|---|---|---|---|---|---|---|---|
| | | Per | (Per | | Day | Day | Day | Day | Day |
| Groups | Test Article | Dose | dose) | | 0 | 14 | 28 | 42 | 58 |
| 1 | 3:1 PSII:CRM₁₉₇ | 45 ug | 50 ug | Proph.¹ | 25 | 100 | 4,800 | 9,600 | |
| | End-linked | | AlOH | 2^{nd} | 25 | 800 | 3,200 | 6,400 | 3,200 |
| | | | | Proph.² | | | | | |
| 2 | 6:1 PSII:CRM₁₉₇ | 45 ug | 50 ug | Proph.¹ | 25 | 800 | 19,200 | 32,000 | |
| | End-linked | | AlOH | 2^{nd} | 25 | 3,200 | 102,400 | 102,400 | 102,400 |
| | | | | Proph.² | | | | | |
| 3 | 10:1 | 45 ug | 50 ug | Proph.¹ | 25 | 800 | 19,200 | 32,000 | |
| | PSII:CRM₁₉₇ | | | | | | | | |
| | End-linked | | AlOH | 2^{nd} | 25 | 3,200 | 51,200 | 51,200 | 102,400 |
| | | | | Proph.² | | | | | |
| 4 | Saline | 45 ug | 50 ug | Proph.¹ | 25 | 25 | 25 | 25 | |
| | | | AlOH | 2^{nd} | 25 | 25 | 25 | 25 | 50 |
| | | | | Proph.² | | | | | |

¹Proph. (Prophylatic immunizations): groups of naïve mice were immunized 3 times, biweekly on Day 0, 14 and 28 with PSII-CRM₁₉₇ conjugates. Sera were collected prior to first immunization and two weeks post each subsequent immunization on Day 14, 28 and 42. Sera were assayed by PSII-specific ELISA and titers are reported above.
²2^{nd} Proph. (secondary prophylatic immunizations): groups of naïve mice were infection with 10\hat{ }4 C. difficile spores and let recovery from all CDI symptoms. On Day 23 post infection, mice were divided into groups of 8 to 10 and PSII-CRM197 conjugates were administrated IM 3 time in a biweekly interval. Sera samples were collected prior to the first immunization and two week post each subsequent immunization on Day 14, 28 and 42. Sera were assayed by PSII-specific ELISA and titers were reported above.

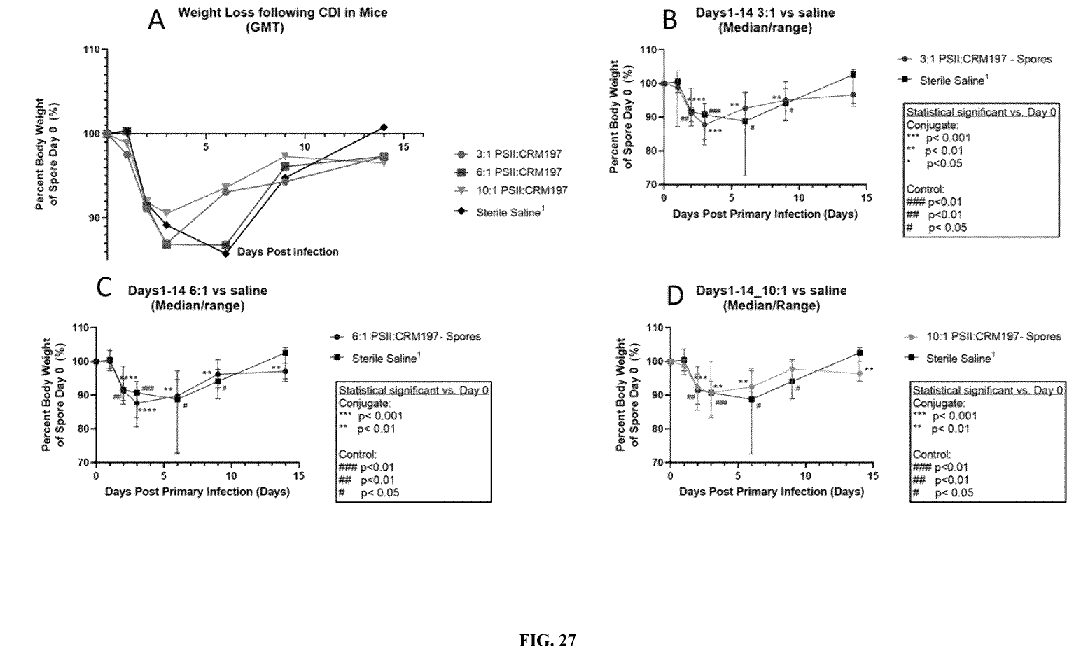

FIG. 27 shows CDI-associated body-weight loss post-C. difficile spore challenge. PANEL A shows weight loss following CDI in mice; PANEL B shows percent body weight changes compared to spore day 0 in mice treated with 3:1 PSII:CRM₁₉₇ over days 1-14; PANEL C shows percent body weight changes compared to spore day 0 in mice treated with 6:1 PSII:CRM₁₉₇ over days 1-14; PANEL D shows percent body weight changes compared to spore day 0 in mice treated with 10:1 PSII:CRM₁₉₇ over days 1-14. For the sterile saline group, one cage of sterile saline control group had no water on day 14, data excluded (n=4). The remaining groups had n=8.

### Example 10: Clinical Trial Study Design

Phase 1 trial: The objective of the phase 1 trial is to optimize formulation and test the safety, tolerability, and immunogenicity of a pharmaceutical composition comprising a C. difficile PSII antigen, a toxoid of toxin A, and a toxoid of toxin B. The phase 1 clinical trial is a 2-arm study comparing the pharmaceutical composition and in two different formulations, each with different adjuvants. The primary endpoint of the phase 1 trial is Ab titers to toxoid A, toxoid B, and PSII; and safety profiles. The secondary endpoint of the phase 1 trial include fecal colonization, CDI incidence, and severity and duration of CDI. Inclusion and exclusion criteria include patients with positive and negative C. difficile colonization. Subjects are examined for differences in immunogenicity. If subjects have had prior CDI, an occurrence threshold is set before the study. The sample size for the phase 1 trial is from 150-200 subjects located in the United States.

Phase 2 clinical trial (main dose determination trial): The objective of the phase 2 clinical trial is to optimize the dose, test the safety, immunogenicity, and efficacy of a pharmaceutical in preventing CDI. The subjected are followed to study the effects of the pharmaceutical composition on long-term CDI. The phase 2 clinical trial is a multi-arm study comparing different doses and formulations of the pharmaceutical composition in addition to standard of care (SOC) treatment. Treatment with the pharmaceutical composition is compared to treatment with SOC only. Primary endpoints include CDI incidence, severity, and duration, as well as safety profiles. Secondary endpoints include Ab titers to toxoid A, toxoid B, and PSII; durability of the immune response; and fecal colonization. Inclusion and exclusion criteria include patients stratified by positive and negative C. difficile colonization. If the subject had a prior CDI, an occurrence threshold is set before the study. Other participation factors include subjects planning for admission to a hospital or nursing home, subjects with family members testing positive for C. difficile colonization, and patient participants who have not recently been on antibiotics. The sample size of the phase 2 clinical trial is from 200-500 subjects located in the United States. Additional subjects in Europe are added as needed.

Phase 3 trial: The objective of the phase 3 clinical trial is to determine the efficacy of a pharmaceutical composition comprising a C. difficile PSII antigen, a toxoid of toxin A, and a toxoid of toxin B in preventing CDI, immunogenicity, safety, and comparative data. The phase 3 study is a 2-arm study comparing the effects of the pharmaceutical composition to PF-06425090 (Pfizer) in primary prevention of CDI; and comparing the effects of the pharmaceutical composition to standard of care in secondary prevention or treatment of CDI. Inclusion and exclusion criteria in the Phase 2 clinical trial are used in the phase 3 trial. A sample size of about 2000 subjects are selected for a multinational study.

### Example 11: Patient Selection Considerations for Clinical Trials

Elderly at-risk patients for primary prevention: Elderly at-risk patients are selected because elderly patients (>age 55) are most at risk, such as subjects who are residents of nursing homes, subjects who have experienced recent hospitalization, or subjects with C. difficile colonization. Elderly patients are selected as a patient group because the subjects are easy to identify (i.e., residents of nursing homes or assisted living homes, recently discharged from hospital, prior use of broad spectrum antibiotics. Enriched patient types allow for a modest study size and lower cost. Due to immunosenescence, elderly patients may require a strong adjuvant. For the phase 3 clinical study, CDI incidence is relatively low, and a larger study is conducted. The risks in selecting an elderly at-risk subject group are the risk of the study being insufficiently powered to show the difference in disease incidence; and that elderly patients may not exhibit robust immune responses.

Adults with C. difficile colonization at risk of CDI (primary and secondary prevention): Adults with C. difficile colonization and at risk of CDI include patients with prior episodes from a pre-determined threshold (e.g., 6 months). Additional at-risk patients include adults with inflammatory bowel disease (IBD) or other GI disorders. Adults with prior CDI have a 1 in 5 chance of recurrence. The subject group includes adults with C. difficile colonization, determined by PCR or NAT, and high-risk adults without prior CDI. The primary endpoint of reduction in CDI is used as a method of differentiating subject groups.

Adults with active disease (primary treatment): Adults with active CDI have the highest unmet need. Fulminant infection has a modest morality rate of about 7%. Treatment with a pharmaceutical composition of the disclosure is added onto standard of care treatment to encourage enrollment. Subjects exhibit a >25% chance of primary CDI, which is greater than recurrent CDI. The study group requires rapid immunogenicity and from only 1 or 2 doses for the clinical benefit of the pharmaceutical composition to be effected within days during the course of the disease. Sufficient immunogenicity must be induced quickly because the first recurrence of CDI occurs within 2-4 weeks of initial occurrence.

### Embodiments

The following non-limiting embodiments provide illustrative examples of the invention, but do not limit the scope of the invention.

Embodiment 1. A composition comprising a cell-surface polysaccharide from Clostridioides difficile (C. difficile), wherein the cell-surface polysaccharide is a PSII antigen enriched from C. difficile, wherein the composition has a total carbohydrate percentage by weight of at least about 40%, and wherein the PSII antigen enriched from C. difficile is at least 90% of the total carbohydrate percentage.

Embodiment 2. The composition of embodiment 1, further comprising a first polypeptide or a first polynucleotide encoding the first polypeptide, wherein the first polypeptide comprises a first toxoid of C. difficile or a fragment thereof.

Embodiment 3. The composition of embodiment 2, wherein the first polypeptide is a toxoid of C. difficile toxin A (TcdA) or a fragment thereof.

Embodiment 4. The composition of embodiment 2 or 3, wherein the first polypeptide is a full-length toxoid of C. difficile toxin A.

Embodiment 5. The composition of any one of embodiments 2-4, further comprising a second polypeptide or a second polynucleotide encoding the second polypeptide, wherein the second polypeptide comprises a second toxoid of C. difficile or a fragment thereof.

Embodiment 6. The composition of embodiment 5, wherein the second polypeptide is a toxoid of C. difficile toxin B (TcdB) or a fragment thereof.

Embodiment 7. The composition of embodiment 6, wherein the second polypeptide is a full-length toxoid of toxin B.

Embodiment 8. The composition of any one of embodiments 1-7, wherein the cell-surface polysaccharide from C. difficile is an anionic cell-surface polysaccharide.

Embodiment 9. The composition of any one of embodiments 1-8, wherein the cell-surface polysaccharide from C. difficile is unsubstituted or substituted.

Embodiment 10. The composition any one of embodiments 1-9, wherein the first polypeptide and the second polypeptide are fused.

Embodiment 11. The composition of any one of embodiments 2-10, wherein the first polypeptide has at least 80% sequence identity to any one of SEQ ID NO: 4-6.

Embodiment 12. The composition of any one of embodiments 5-11, wherein the second polypeptide has at least 80% sequence identity to SEQ ID NO: 1-3 or 7-40.

Embodiment 13. The composition of any one of embodiments 2-12, wherein a ratio of the cell-surface polysaccharide from C. difficile to the first polypeptide is from about 10:1 to about 1:10.

Embodiment 14. The composition of any one of embodiments 5-13, wherein a ratio of the cell-surface polysaccharide from C. difficile to the second polypeptide is about 10:1 to about 1:10.

Embodiment 15. The composition of any one of embodiments 1-14, wherein the PSII antigen is PSII, a pharmaceutically acceptable salt, or an immunogenic fragment thereof.

Embodiment 16. The composition of embodiment 15, wherein the PSII, the pharmaceutically acceptable salt, or the immunogenic fragment thereof comprises a phosphate moiety.

Embodiment 17. The composition any one of embodiments 5-16, wherein the cell-surface polysaccharide is not conjugated to the first polypeptide or the second polypeptide.

Embodiment 18. The composition of any one of embodiments 1-17, wherein the PSII is a polysaccharide of Formula (I):

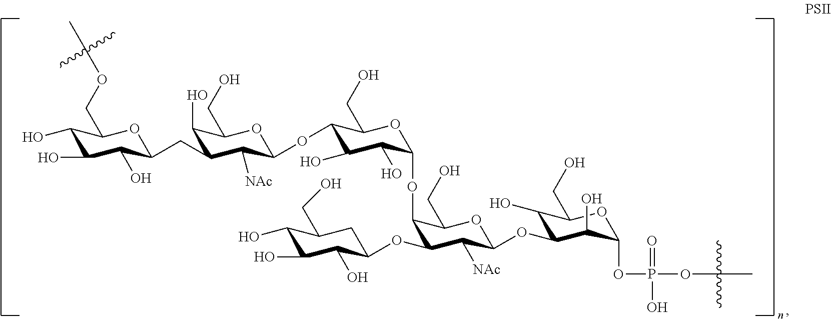

wherein n is an integer of from 1 to 100.

Embodiment 19. The composition of any one of embodiments 1-18, wherein the cell-surface polysaccharide has a molecular weight of from about 5 kDa to about 10 kDa.

Embodiment 20. The composition of any one of embodiments 1-19, wherein the cell-surface polysaccharide has a molecular weight of about 8.8 kDa.

Embodiment 21. The composition of any one of embodiments 1-20, further comprising an adjuvant.

Embodiment 22. The composition of embodiment 21, wherein the adjuvant comprises an aluminum-based adjuvant.

Embodiment 23. The composition of embodiment 21, wherein the adjuvant comprises aluminum hydroxide.

Embodiment 24. The composition of embodiment 21, wherein the adjuvant comprises aluminum phosphate.

Embodiment 25. The composition of embodiment 21, wherein the adjuvant comprises delta inulin microparticles.

Embodiment 26. The composition of any one of embodiments 1-25, wherein the PSII antigen is conjugated to a pharmaceutically acceptable carrier.

Embodiment 27. The composition of embodiment 26, wherein the pharmaceutically acceptable carrier is a mutant of a diphtheria toxin.

Embodiment 28. The composition of embodiment 26, wherein the pharmaceutically acceptable carrier is CRM₁₉₇.

Embodiment 29. The composition of any one of embodiments 26-28, wherein the PSII antigen is conjugated to the pharmaceutically acceptable carrier by a chemical linker.

Embodiment 30. The composition of embodiment 29, wherein the chemical linker comprises a thiosuccinimide.

Embodiment 31. The composition of embodiment 29, wherein the chemical linker comprises a thioester.

Embodiment 32. The composition of any one of embodiments 1-31, wherein the PSII antigen is conjugated to CRM₁₉₇ and has a molecular weight of from about 100 kDa to about 1000 kDa.

Embodiment 33. The composition of any one of embodiments 1-31, wherein the PSII antigen is conjugated to CRM₁₉₇ and has a molecular weight of from about 100 kDa to about 350 kDa.

Embodiment 34. The composition of any one of embodiments 1-33, wherein the PSII antigen is a native cell-surface polysaccharide from C. difficile.

Embodiment 35. The composition of any one of embodiments 1-34, wherein the PSII antigen is non-synthetic.

Embodiment 36. The composition of any one of embodiments 1-35, wherein the PSII antigen is purified from one or more strains of C. difficile.

Embodiment 37. The composition of any one of embodiments 1-36, wherein the PSII antigen is from a cell-surface extract (CSE) of one or more strains of C. difficile.

Embodiment 38. The composition of any one of embodiments 1-37, wherein the composition comprises less than about 20% by weight of a polypeptide from C. difficile.

Embodiment 39. The composition of any one of embodiments 1-38, wherein the C. difficile is ribotype 001, 003, 012, 014, 027, 036, 106, MOH 900, or MOH 718.

Embodiment 40. The composition of any one of embodiments 1-39, wherein the composition is an immunogenic composition or a vaccine.

Embodiment 41. The composition of embodiment 40, wherein the composition induces neutralization titers against the PSII antigen.

Embodiment 42. The composition of embodiment 40 or 41, wherein the composition induces neutralization titers against C. difficile toxin A or C. difficile toxin B.

Embodiment 43. The composition of embodiment 40 or 41, wherein the composition induces neutralization titers against C. difficile toxin A and C. difficile toxin B.

Embodiment 44. The composition of any one of embodiments 1-43, wherein the composition comprises less than about 5% by weight of an impurity relative to the total weight of the PSII antigen.

Embodiment 45. The composition of embodiment 44, wherein the impurity is a peptidoglycan, a protein, a nucleic acid, a saccharide, or a combination thereof.

Embodiment 46. The composition of embodiment 44 or 45, wherein the impurity is a C. difficile impurity.

Embodiment 47. The composition of any one of embodiments 44-46, wherein the impurity is a nucleic acid.

Embodiment 48. The composition of any one of embodiments 44-46, wherein the impurity is a saccharide.

Embodiment 49. The composition of any one of embodiments 44-46, wherein the impurity is derived from a cell-surface extract of C. difficile.

Embodiment 50. The composition of any one of embodiments 1-49, wherein the composition comprises less than about 5% by weight of a peptidoglycan impurity relative to the total weight of the PSII antigen.

Embodiment 51. The composition of any one of embodiments 1-50, wherein the composition comprises less than about 5% by weight of a protein impurity relative to the total weight of the PRI antigen.

Embodiment 52. The composition of any one of embodiments 1-51, wherein the composition comprises less than about 5% by weight of a nucleic acid impurity relative to the total weight of the PSII antigen.

Embodiment 53. The composition of any one of embodiments 1-52, wherein the composition comprises less than about 5% by weight of a saccharide impurity relative to the total weight of the PRI antigen.

Embodiment 54. The composition of any one of embodiments 1-53, wherein the composition comprises less than about 5% by weight of a mixture of impurities relative to the total weight of the PSII antigen, wherein the mixture of impurities is selected from the group consisting of
(a) a mixture of nucleic acid and protein impurities relative to the total weight of the PRI antigen;
(b) a mixture of nucleic acid and peptidoglycan impurities relative to the total weight of the PSII antigen;
(c) a mixture of peptidoglycan and protein impurities relative to the total weight of the PRI antigen;
(d) a mixture of saccharide and protein impurities relative to the total weight of the PRI antigen;
(e) a mixture of peptidoglycan and saccharide impurities; and
(f) a mixture of nucleic acid and saccharide impurities relative to the total weight of the PSII antigen.

Embodiment 55. The composition of any one of embodiments 1-54, wherein the composition comprises less than about 5% by weight of a mixture of impurities relative to the total weight of the PSII antigen, wherein the mixture of impurities is selected from the group consisting of
(a) a mixture of nucleic acid, protein and peptidoglycan impurities relative to the total weight of the PSII antigen;
(b) a mixture of saccharide, protein and peptidoglycan impurities relative to the total weight of the PSII antigen;
(c) a mixture of nucleic acid, protein and saccharide impurities relative to the total weight of the PSII antigen; and
(d) a mixture of nucleic acid, peptidoglycan and saccharide impurities relative to the total weight of the PRI antigen.

Embodiment 56. The composition of any one of embodiments 1-55, comprising less than about 5% by weight of a mixture of nucleic acid, peptidoglycan, saccharide and protein impurities relative to the total weight of the PSII antigen.

Embodiment 57. The composition of any one of embodiments 1-56, comprising at least 5% by weight of the PRI antigen.

Embodiment 58. A composition comprising:
(a) a cell-surface polysaccharide of Clostridioides difficile (C. difficile); and
(b) a first polypeptide comprising a carrier protein;
   wherein the carrier protein and the cell-surface polysaccharide of C. difficile are present in the composition at a ratio of less than about 10:1 to about 1:3.

Embodiment 59. The composition of embodiment 58, further comprising a second polypeptide or a first polynucleotide encoding the second polypeptide, wherein the second polypeptide comprises a first toxoid of C. difficile or a fragment thereof.

Embodiment 60. The composition of embodiment 59, wherein the second polypeptide is a toxoid of C. difficile toxin A (TcdA) or a fragment thereof.

Embodiment 61. The composition of embodiment 59 or 60, wherein the second polypeptide is a full-length toxoid of toxin A.

Embodiment 62. The composition of any one of embodiments 58-61, further comprising a third polypeptide or a second polynucleotide encoding the third polypeptide, wherein the third polypeptide comprises a second toxoid of C. difficile or a fragment thereof.

Embodiment 63. The composition of embodiment 62, wherein the third polypeptide is a toxoid of C. difficile toxin B (TcdB) or a fragment thereof.

Embodiment 64. The composition of embodiment 62 or 63, wherein the third polypeptide is a full-length toxoid of toxin B.

Embodiment 65. The composition of any one of embodiments 58-64, wherein the cell-surface polysaccharide from C. difficile is an anionic cell-surface polysaccharide.

Embodiment 66. The composition of any one of embodiments 58-65, wherein the cell-surface polysaccharide from C. difficile is unsubstituted or substituted.

Embodiment 67. The composition of any one of embodiments 62-66, wherein the second polypeptide and the third polypeptide are fused.

Embodiment 68. The composition of any one of embodiments 59-67, wherein the second polypeptide has at least 80% sequence identity to any one of SEQ ID NO: 4-6.

Embodiment 69. The composition of any one of embodiments 62-68, wherein the third polypeptide has at least 80% sequence identity to SEQ ID NO: 1-3 or 7-40.

Embodiment 70. The composition of any one of embodiments 59-69, wherein a ratio of the cell-surface polysaccharide from C. difficile to the second polypeptide is from about 10:1 to about 1:10.

Embodiment 71. The composition of any one of embodiments 62-70, wherein a ratio of the cell-surface polysaccharide from C. difficile to the third polypeptide is about 10:1 to about 1:10.

Embodiment 72. The composition of any one of embodiments 58-71, wherein the cell-surface polysaccharide from C. difficile is PSII, a pharmaceutically acceptable salt, or an immunogenic fragment thereof.

Embodiment 73. The composition of embodiment 72, wherein the PSII or a pharmaceutically acceptable salt or immunogenic fragment thereof comprises a phosphate moiety.

Embodiment 74. The composition of any one of embodiments 62-73, wherein the cell-surface polysaccharide is enriched from C. difficile and is not conjugated to the second polypeptide or the third polypeptide.

Embodiment 75. The composition of any one of embodiments 58-74, wherein the cell-surface polysaccharide from C. difficile is an enriched cell-surface polysaccharide from C. difficile.

Embodiment 76. The composition of embodiment 72 or 73, wherein the PSII is a polysaccharide of Formula (I):

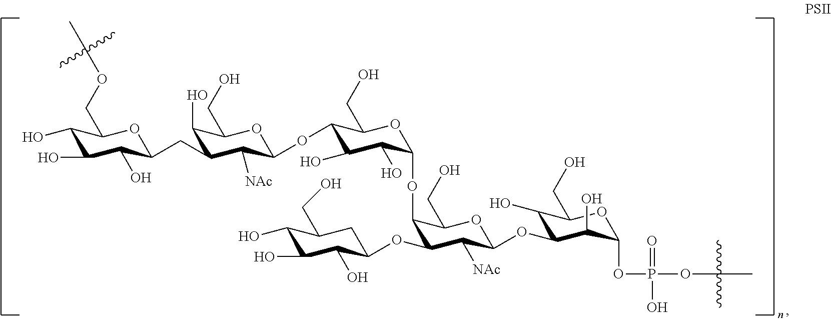

wherein n is an integer of from 1 to 100.

Embodiment 77. The composition of any one of embodiments 58-76, wherein the cell-surface polysaccharide has a molecular weight of from about 5 kDa to about 10 kDa.

Embodiment 78. The composition of any one of embodiments 58-77, wherein the cell-surface polysaccharide has a molecular weight of about 8.8 kDa.

Embodiment 79. The composition of any one of embodiments 58-78, further comprising an adjuvant.

Embodiment 80. The composition of embodiment 79, wherein the adjuvant comprises an aluminum-based adjuvant.

Embodiment 81. The composition of embodiment 79, wherein the adjuvant comprises aluminum hydroxide.

Embodiment 82. The composition of embodiment 79, wherein the adjuvant comprises aluminum phosphate.

Embodiment 83. The composition of embodiment 79, wherein the adjuvant comprises delta inulin microparticles.

Embodiment 84. The composition of any one of embodiments 58-83, wherein the cell-surface polysaccharide of C. difficile is conjugated to the carrier protein.

Embodiment 85. The composition of embodiment 84, wherein the carrier protein is a mutant of a diphtheria toxin.

Embodiment 86. The composition of embodiment 84, wherein the carrier protein is CRM₁₉₇.

Embodiment 87. The composition of any one of embodiments 84-86, wherein the cell-surface polysaccharide of C. difficile is conjugated to the carrier protein by a chemical linker.

Embodiment 88. The composition of embodiment 87, wherein the chemical linker comprises a thiosuccinimide.

Embodiment 89. The composition of embodiment 87, wherein the chemical linker comprises a thioester.

Embodiment 90. The composition of any one of embodiments 58-89, wherein the cell-surface polysaccharide of C. difficile is conjugated to CRM₁₉₇ and has a molecular weight of from about 100 kDa to about 1000 kDa.

Embodiment 91. The composition of embodiment 90, wherein the cell-surface polysaccharide of C. difficile is conjugated to CRM₁₉₇ and has a molecular weight of from about 100 kDa to about 350 kDa.

Embodiment 92. The composition of any one of embodiments 58-91, wherein the cell-surface polysaccharide from C. difficile is a native cell-surface polysaccharide from C. difficile.

Embodiment 93. The composition of any one of embodiments 58-92, wherein the cell-surface polysaccharide from C. difficile is non-synthetic.

Embodiment 94. The composition of any one of embodiments 58-93, wherein the cell-surface polysaccharide from C. difficile is purified from one or more strains of C. difficile.

Embodiment 95. The composition of any one of embodiments 58-94, wherein the cell-surface polysaccharide from C. difficile is from a cell-surface extract (CSE) of one or more strains of C. difficile.

Embodiment 96. The composition of any one of embodiments 58-95, wherein the composition or pharmaceutical composition comprises less than 20% by weight of a polypeptide from C. difficile.

Embodiment 97. The composition of any one of embodiments 58-96, wherein the C. difficile is ribotype 001, 003, 012, 014, 027, 036, 106, MOH 900, or MOH 718.

Embodiment 98. The composition of any one of embodiments 58-97, wherein the composition is an immunogenic composition or a vaccine.

Embodiment 99. The composition of embodiment 98, wherein the composition induces neutralization titers against the PSII antigen.

Embodiment 100. The composition of embodiment 98 or 99, wherein the composition induces neutralization titers against C. difficile toxin A and/or C. difficile toxin B.

Embodiment 101. The composition of any one of embodiments 58-100, wherein the composition comprises less than about 5% by weight of an impurity relative to the total weight of the cell-surface polysaccharide of C. difficile.

Embodiment 102. The composition of embodiment 101, wherein the impurity is a peptidoglycan, a protein, a nucleic acid, a saccharide, or a combination thereof.

Embodiment 103. The composition of embodiment 101 or 102, wherein the impurity is a C. difficile impurity.

Embodiment 104. The composition of any one of embodiments 101-103, wherein the impurity is a nucleic acid.

Embodiment 105. The composition of any one of embodiments 101-103, wherein the impurity is a saccharide.

Embodiment 106. The composition of any one of embodiments 101-103, wherein the impurity is derived from a cell-surface extract of C. difficile.

Embodiment 107. The composition of any one of embodiments 58-102, wherein the composition comprises less than about 5% by weight of a peptidoglycan impurity relative to the total weight of the cell-surface polysaccharide.

Embodiment 108. The composition of any one of embodiments 58-102, wherein the composition comprises less than about 5% by weight of a protein impurity relative to the total weight of the cell-surface polysaccharide.

Embodiment 109. The composition of any one of embodiments 58-102, wherein the composition comprises less than about 5% by weight of a nucleic acid impurity relative to the total weight of the cell-surface polysaccharide.

Embodiment 110. The composition of any one of embodiments 58-102, wherein the composition comprises less than about 5% by weight of a saccharide impurity relative to the total weight of the cell-surface polysaccharide.

Embodiment 111. Embodiment 199. The composition of any one of embodiments 58-101, wherein the composition comprises less than about 5% by weight of a mixture of impurities relative to the total weight of the cell-surface polysaccharide, wherein the mixture of impurities is selected from the group consisting of
(a) a mixture of nucleic acid and protein impurities relative to the total weight of the cell-surface polysaccharide;
(b) a mixture of nucleic acid and peptidoglycan impurities relative to the total weight of the cell-surface polysaccharide;
(c) a mixture of peptidoglycan and protein impurities relative to the total weight of the cell-surface polysaccharide;
(d) a mixture of saccharide and protein impurities relative to the total weight of the cell-surface polysaccharide;
(e) a mixture of peptidoglycan and saccharide impurities; and
(f) a mixture of nucleic acid and saccharide impurities relative to the total weight of the cell-surface polysaccharide.

Embodiment 112. The composition of any one of embodiments 58-101, wherein the composition comprises less than about 5% by weight of a mixture of impurities relative to the total weight of the cell-surface polysaccharide, wherein the mixture of impurities is selected from the group consisting of
(a) a mixture of nucleic acid, protein and peptidoglycan impurities relative to the total weight of the cell-surface polysaccharide;
(b) a mixture of saccharide, protein and peptidoglycan impurities relative to the total weight of the cell-surface polysaccharide;
(c) a mixture of nucleic acid, protein and saccharide impurities relative to the total weight of the cell-surface polysaccharide; and
(d) a mixture of nucleic acid, peptidoglycan and saccharide impurities relative to the total weight of the cell-surface polysaccharide.

Embodiment 113. The composition of any one of embodiments 58-112, comprising less than about 5% by weight of a mixture of nucleic acid, peptidoglycan, saccharide, and protein impurities relative to the total weight of the cell-surface polysaccharide.

Embodiment 114. The composition of any one of embodiments 58-113, comprising at least 5% by weight of the cell-surface polysaccharide from C. difficile.

Embodiment 115. A composition comprising:
(a) a cell-surface polysaccharide of Clostridioides difficile (C. difficile); and
(b) a first polypeptide comprising a carrier protein derived from an organism other than C. difficile;
   wherein the carrier protein and the cell-surface polysaccharide are present in the composition at a ratio of from about 10:1 to about 1:10.

Embodiment 116. The composition of embodiment 115, further comprising a second polypeptide or a first polynucleotide encoding the second polypeptide, wherein the second polypeptide comprises a first toxoid of C. difficile or a fragment thereof.

Embodiment 117. The composition of embodiment 116, wherein the second polypeptide is a toxoid of C. difficile toxin A (TcdA) or a fragment thereof.

Embodiment 118. The composition of embodiment 116 or 117, wherein the second polypeptide is a full-length toxoid of toxin A.

Embodiment 119. The composition of any one of embodiments 115-118, further comprising a third polypeptide or a second polynucleotide encoding the third polypeptide, wherein the third polypeptide comprises a second toxoid of C. difficile or a fragment thereof.

Embodiment 120. The composition of embodiment 119, wherein the third polypeptide is a toxoid of C. difficile toxin B (TcdB) or a fragment thereof.

Embodiment 121. The composition of embodiment 119 or 120, wherein the third polypeptide is a full-length toxoid of toxin B.

Embodiment 122. The composition of any one of embodiments 115-121, wherein the cell-surface polysaccharide from C. difficile is an anionic cell-surface polysaccharide.

Embodiment 123. The composition of any one of embodiments 115-122, wherein the cell-surface polysaccharide from C. difficile is unsubstituted or substituted.

Embodiment 124. The composition of any one of embodiments 116-123, wherein the second polypeptide and the third polypeptide are fused.

Embodiment 125. The composition of any one of embodiments 116-124, wherein the second polypeptide has at least 80% sequence identity to any one of SEQ ID NO: 4-6.

Embodiment 126. The composition of any one of embodiments 116-125, wherein the third polypeptide has at least 80% sequence identity to SEQ ID NO: 1-3 or 7-40.

Embodiment 127. The composition of any one of embodiments 116-126, wherein a ratio of the cell-surface polysaccharide from C. difficile to the second polypeptide is from about 10:1 to about 1:10.

Embodiment 128. The composition of any one of embodiments 119-127, wherein a ratio of the cell-surface polysaccharide from C. difficile to the third polypeptide is about 10:1 to about 1:10.

Embodiment 129. The composition of any one of embodiments 115-128, wherein the cell-surface polysaccharide from C. difficile is PSII, a pharmaceutically acceptable salt, or an immunogenic fragment thereof.

Embodiment 130. The composition of embodiment 129, wherein the PSII or a pharmaceutically acceptable salt or immunogenic fragment thereof comprises a phosphate moiety.

Embodiment 131. The composition of any one of embodiments 115-130, wherein the cell-surface polysaccharide is enriched from C. difficile and is not conjugated to the second polypeptide or the third polypeptide.

Embodiment 132. The composition of any one of embodiments 115-131, wherein the cell-surface polysaccharide from C. difficile is an enriched cell-surface polysaccharide from C. difficile.

Embodiment 133. The composition of embodiment 129 or 130, wherein the PRI is a polysaccharide of Formula (I):

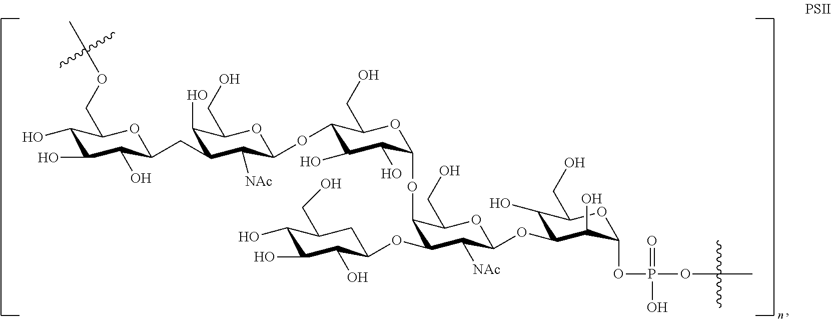

wherein n is an integer of from 1 to 100.

Embodiment 134. The composition of any one of embodiments 115-133, wherein the cell-surface polysaccharide has a molecular weight of from about 5 kDa to about 10 kDa.

Embodiment 135. The composition of any one of embodiments 115-134, wherein the cell-surface polysaccharide has a molecular weight of about 8.8 kDa.

Embodiment 136. The composition of any one of embodiments 115-135, further comprising an adjuvant.

Embodiment 137. The composition of embodiment 136, wherein the adjuvant comprises an aluminum-based adjuvant.

Embodiment 138. The composition of embodiment 136, wherein the adjuvant comprises aluminum hydroxide.

Embodiment 139. The composition of embodiment 136, wherein the adjuvant comprises aluminum phosphate.

Embodiment 140. The composition of embodiment 136, wherein the adjuvant comprises delta inulin microparticles.

Embodiment 141. The composition of any one of embodiments 115-140, wherein the cell-surface polysaccharide of C. difficile is conjugated to the carrier protein.

Embodiment 142. The composition of embodiment 141, wherein the carrier protein is a mutant of a diphtheria toxin.

Embodiment 143. The composition of embodiment 141, wherein the carrier protein is CRM₁₉₇.

Embodiment 144. The composition of any one of embodiments 141-143, wherein the cell-surface polysaccharide of C. difficile is conjugated to the carrier protein by a chemical linker.

Embodiment 145. The composition of embodiment 144, wherein the chemical linker comprises a thiosuccinimide.

Embodiment 146. The composition of embodiment 144, wherein the chemical linker comprises a thioester.

Embodiment 147. The composition of any one of embodiments 115-146, wherein the cell-surface polysaccharide of C. difficile is conjugated to CRM₁₉₇ and has a molecular weight of from about 100 kDa to about 1000 kDa.

Embodiment 148. The composition of embodiment 147, wherein the cell-surface polysaccharide of C. difficile is conjugated to CRM₁₉₇ and has a molecular weight of from about 100 kDa to about 350 kDa.

Embodiment 149. The composition of any one of embodiments 115-148, wherein the cell-surface polysaccharide from C. difficile is a native cell-surface polysaccharide from C. difficile.

Embodiment 150. The composition of any one of embodiments 115-149, wherein the cell-surface polysaccharide from C. difficile is non-synthetic.

Embodiment 151. The composition of any one of embodiments 115-150, wherein the cell-surface polysaccharide from C. difficile is purified from one or more strains of C. difficile.

Embodiment 152. The composition of any one of embodiments 115-151, wherein the cell-surface polysaccharide from C. difficile is from a cell-surface extract (CSE) of one or more strains of C. difficile.

Embodiment 153. The composition of any one of embodiments 115-152, wherein the composition or pharmaceutical composition comprises less than 20% by weight of a polypeptide from C. difficile.

Embodiment 154. The composition of any one of embodiments 115-153, wherein the C. difficile is ribotype 001, 003, 012, 014, 027, 036, 106, MOH 900, or MOH 718.

Embodiment 155. The composition of any one of embodiments 115-154, wherein the composition is an immunogenic composition or a vaccine.

Embodiment 156. The composition of embodiment 155, wherein the composition induces neutralization titers against the PSII antigen.

Embodiment 157. The composition of embodiment 155 or 156, wherein the composition induces neutralization titers against C. difficile toxin A and/or C. difficile toxin B.

Embodiment 158. The composition of any one of embodiments 115-157, wherein the composition comprises less than about 5% by weight of an impurity relative to the total weight of the cell-surface polysaccharide of C. difficile.

Embodiment 159. The composition of embodiment 158, wherein the impurity is a peptidoglycan, a protein, a nucleic acid, a saccharide, or a combination thereof.

Embodiment 160. The composition of embodiment 158 or 159, wherein the impurity is a C. difficile impurity.

Embodiment 161. The composition of any one of embodiments 158-160, wherein the impurity is a nucleic acid.

Embodiment 162. The composition of any one of embodiments 158-160, wherein the impurity is a saccharide.

Embodiment 163. The composition of any one of embodiments 158-160, wherein the impurity is derived from a cell-surface extract of C. difficile.

Embodiment 164. The composition of any one of embodiments 115-163, wherein the composition comprises less than about 5% by weight of a peptidoglycan impurity relative to the total weight of the cell-surface polysaccharide.

Embodiment 165. The composition of any one of embodiments 115-163, wherein the composition comprises less than about 5% by weight of a protein impurity relative to the total weight of the cell-surface polysaccharide.

Embodiment 166. The composition of any one of embodiments 115-163, wherein the composition comprises less than about 5% by weight of a nucleic acid impurity relative to the total weight of the cell-surface polysaccharide.

Embodiment 167. The composition of any one of embodiments 115-163, wherein the composition comprises less than about 5% by weight of a saccharide impurity relative to the total weight of the cell-surface polysaccharide.

Embodiment 168. The composition of any one of embodiments 115-159, wherein the composition comprises less than about 5% by weight of a mixture of impurities relative to the total weight of the cell-surface polysaccharide, wherein the mixture of impurities is selected from the group consisting of
(a) a mixture of nucleic acid and protein impurities relative to the total weight of the cell-surface polysaccharide;
(b) a mixture of nucleic acid and peptidoglycan impurities relative to the total weight of the cell-surface polysaccharide;
(c) a mixture of peptidoglycan and protein impurities relative to the total weight of the cell-surface polysaccharide;
(d) a mixture of saccharide and protein impurities relative to the total weight of the cell-surface polysaccharide;
(e) a mixture of peptidoglycan and saccharide impurities; and
(f) a mixture of nucleic acid and saccharide impurities relative to the total weight of the cell-surface polysaccharide.

Embodiment 169. The composition of any one of embodiments 115-159, wherein the composition comprises less than about 5% by weight of a mixture of impurities relative to the total weight of the cell-surface polysaccharide, wherein the mixture of impurities is selected from the group consisting of
(a) a mixture of nucleic acid, protein and peptidoglycan impurities relative to the total weight of the cell-surface polysaccharide;
(b) a mixture of saccharide, protein and peptidoglycan impurities relative to the total weight of the cell-surface polysaccharide;
(c) a mixture of nucleic acid, protein and saccharide impurities relative to the total weight of the cell-surface polysaccharide; and
(d) a mixture of nucleic acid, peptidoglycan and saccharide impurities relative to the total weight of the cell-surface polysaccharide.

Embodiment 170. The composition of any one of embodiments 115-169, comprising less than about 5% by weight of a mixture of nucleic acid, peptidoglycan, saccharide, and protein impurities relative to the total weight of the cell-surface polysaccharide.

Embodiment 171. The composition of any one of embodiments 115-170, comprising at least 5% by weight of the cell-surface polysaccharide from C. difficile.

Embodiment 172. A pharmaceutical composition comprising the composition of any one of embodiments 1-171.

Embodiment 173. A pharmaceutical composition comprising:
(a) a cell-surface polysaccharide enriched from Clostridioides difficile (C. difficile);
(b) a first polypeptide or a first polynucleotide encoding the first polypeptide, wherein the first polypeptide comprises a first toxoid of C. difficile or a fragment thereof;
(c) a second polypeptide or a second polynucleotide encoding the second polypeptide, wherein the second polypeptide comprises a second toxoid of C. difficile or a fragment thereof; and
(d) a pharmaceutically acceptable carrier;
   wherein the pharmaceutical composition has a total carbohydrate percentage,
wherein at least about 90% of the total carbohydrate percentage is PSII.

Embodiment 174. The pharmaceutical composition of embodiment 173, wherein the first polypeptide is a toxoid of C. difficile toxin A (TcdA) or a fragment thereof.

Embodiment 175. The pharmaceutical composition of embodiment 173-174, wherein the first polypeptide is a full-length toxoid of C. difficile toxin A.

Embodiment 176. The pharmaceutical composition of any one of embodiments 173-175, wherein the second polypeptide is a toxoid of C. difficile toxin B (TcdB) or a fragment thereof.

Embodiment 177. The pharmaceutical composition of any one of embodiments 173-176, wherein the second polypeptide is a full-length toxoid of toxin B.

Embodiment 178. The pharmaceutical composition of any one of embodiments 173-177, wherein the cell-surface polysaccharide from C. difficile is an anionic cell-surface polysaccharide.

Embodiment 179. The pharmaceutical composition of any one of embodiments 173-178, wherein the cell-surface polysaccharide from C. difficile is unsubstituted or substituted.

Embodiment 180. The pharmaceutical composition any one of embodiments 173-179, wherein the first polypeptide and the second polypeptide are fused.

Embodiment 181. The pharmaceutical composition of any one of embodiments 173-180, wherein the first polypeptide has at least 80% sequence identity to any one of SEQ ID NO: 4-6.

Embodiment 182. The pharmaceutical composition of any one of embodiments 173-181, wherein the second polypeptide has at least 80% sequence identity to SEQ ID NO: 1-3 or 7-40.

Embodiment 183. The pharmaceutical composition of any one of embodiments 173-182, wherein a ratio of the cell-surface polysaccharide from C. difficile to the first polypeptide is from about 10:1 to about 1:10.

Embodiment 184. The pharmaceutical composition of any one of embodiments 173-183, wherein a ratio of the cell-surface polysaccharide from C. difficile to the second polypeptide is about 10:1 to about 1:10.

Embodiment 185. The pharmaceutical composition of any one of embodiments 173-184, wherein the cell-surface polysaccharide enriched from C. difficile is PSII, a pharmaceutically acceptable salt, or an immunogenic fragment thereof.

Embodiment 186. The pharmaceutical composition of embodiment 185, wherein the PSII, the pharmaceutically acceptable salt, or the immunogenic fragment thereof comprises a phosphate moiety.

Embodiment 187. The pharmaceutical composition any one of embodiments 173-186, wherein the cell-surface polysaccharide is not conjugated to the first polypeptide or the second polypeptide.

Embodiment 188. The pharmaceutical composition of any one of embodiments 173-187, wherein the PSII is a polysaccharide of Formula (I):

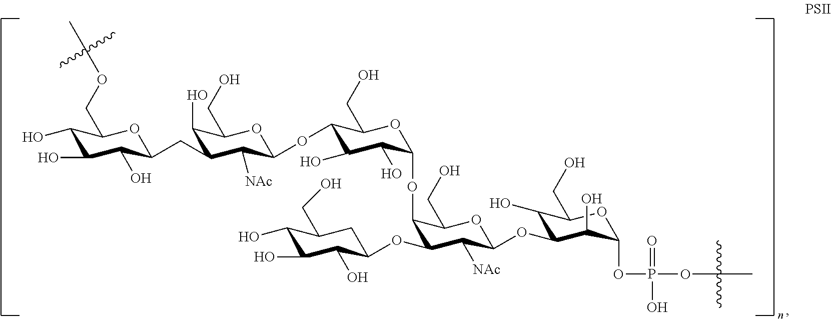

wherein n is an integer of from 1 to 100.

Embodiment 189. The pharmaceutical composition of any one of embodiments 173-188, wherein the cell-surface polysaccharide has a molecular weight of from about 5 kDa to about 10 kDa.

Embodiment 190. The pharmaceutical composition of any one of embodiments 173-189, wherein the cell-surface polysaccharide has a molecular weight of about 8.8 kDa.

Embodiment 191. The pharmaceutical composition of any one of embodiments 173-190, further comprising an adjuvant.

Embodiment 192. The pharmaceutical composition of embodiment 191, wherein the adjuvant comprises an aluminum-based adjuvant.

Embodiment 193. The pharmaceutical composition of embodiment 191, wherein the adjuvant comprises aluminum hydroxide.

Embodiment 194. The pharmaceutical composition of embodiment 191, wherein the adjuvant comprises aluminum phosphate.

Embodiment 195. The pharmaceutical composition of embodiment 191, wherein the adjuvant comprises delta inulin microparticles.

Embodiment 196. The pharmaceutical composition of any one of embodiments 173-195, wherein the cell-surface polysaccharide enriched from C. difficile is conjugated to a pharmaceutically acceptable carrier.

Embodiment 197. The pharmaceutical composition of embodiment 196, wherein the pharmaceutically acceptable carrier is a mutant of a diphtheria toxin.

Embodiment 198. The pharmaceutical composition of embodiment 196, wherein the pharmaceutically acceptable carrier is CRM₁₉₇.

Embodiment 199. The pharmaceutical composition of any one of embodiments 196-198, wherein the cell-surface polysaccharide enriched from C. difficile is conjugated to the pharmaceutically acceptable carrier by a chemical linker.

Embodiment 200. The pharmaceutical composition of embodiment 199, wherein the chemical linker comprises a thiosuccinimide.

Embodiment 201. The pharmaceutical composition of embodiment 199, wherein the chemical linker comprises a thioester.

Embodiment 202. The pharmaceutical composition of any one of embodiments 173-201, wherein the cell-surface polysaccharide enriched from C. difficile is conjugated to CRM₁₉₇ and has a molecular weight of from about 100 kDa to about 1000 kDa.

Embodiment 203. The pharmaceutical composition of any one of embodiments 173-201, wherein the cell-surface polysaccharide enriched from C. difficile is conjugated to CRM₁₉₇ and has a molecular weight of from about 100 kDa to about 350 kDa.

Embodiment 204. The pharmaceutical composition of any one of embodiments 173-203, wherein the cell-surface polysaccharide enriched from C. difficile is a native cell-surface polysaccharide from C. difficile.

Embodiment 205. The pharmaceutical composition of any one of embodiments 173-204, wherein the cell-surface polysaccharide enriched from C. difficile is non-synthetic.

Embodiment 206. The pharmaceutical composition of any one of embodiments 173-205, wherein the cell-surface polysaccharide enriched from C. difficile is purified from one or more strains of C. difficile.

Embodiment 207. The pharmaceutical composition of any one of embodiments 173-206, wherein the cell-surface polysaccharide enriched from C. difficile is from a cell-surface extract (CSE) of one or more strains of C. difficile.

Embodiment 208. The pharmaceutical composition of any one of embodiments 173-207, wherein the pharmaceutical composition comprises less than about 20% by weight of a polypeptide from C. difficile.

Embodiment 209. The pharmaceutical composition of any one of embodiments 173-208, wherein the C. difficile is ribotype 001, 003, 012, 014, 027, 036, 106, MOH 900, or MOH 718.

Embodiment 210. The pharmaceutical composition of any one of embodiments 173-209, wherein the pharmaceutical composition is an immunogenic pharmaceutical composition or a vaccine.

Embodiment 211. The pharmaceutical composition of embodiment 210, wherein the pharmaceutical composition induces neutralization titers against the PSII antigen.

Embodiment 212. The pharmaceutical composition of embodiment 210 or 211, wherein the pharmaceutical composition induces neutralization titers against C. difficile toxin A or C. difficile toxin B.

Embodiment 213. The pharmaceutical composition of embodiment 210 or 211, wherein the pharmaceutical composition induces neutralization titers against C. difficile toxin A and C. difficile toxin B.

Embodiment 214. The pharmaceutical composition of any one of embodiments 173-213, wherein the pharmaceutical composition comprises less than about 5% by weight of an impurity relative to the total weight of the cell-surface polysaccharide enriched from C. difficile.

Embodiment 215. The pharmaceutical composition of embodiment 214, wherein the impurity is a peptidoglycan, a protein, a nucleic acid, a saccharide, or a combination thereof.

Embodiment 216. The pharmaceutical composition of embodiment 214 or 215, wherein the impurity is a C. difficile impurity.

Embodiment 217. The pharmaceutical composition of any one of embodiments 214-216, wherein the impurity is a nucleic acid.

Embodiment 218. The pharmaceutical composition of any one of embodiments 214-216, wherein the impurity is a saccharide.

Embodiment 219. The pharmaceutical composition of any one of embodiments 214-216, wherein the impurity is derived from a cell-surface extract of C. difficile.

Embodiment 220. The pharmaceutical composition of any one of embodiments 173-219, wherein the pharmaceutical composition comprises less than about 5% by weight of a peptidoglycan impurity relative to the total weight of the cell-surface polysaccharide enriched from C. difficile.

Embodiment 221. The pharmaceutical composition of any one of embodiments 173-220, wherein the pharmaceutical composition comprises less than about 5% by weight of a protein impurity relative to the total weight of the cell-surface polysaccharide enriched from C. difficile.

Embodiment 222. The pharmaceutical composition of any one of embodiments 173-221, wherein the pharmaceutical composition comprises less than about 5% by weight of a nucleic acid impurity relative to the total weight of the cell-surface polysaccharide enriched from C. difficile.

Embodiment 223. The pharmaceutical composition of any one of embodiments 173-222, wherein the pharmaceutical composition comprises less than about 5% by weight of a saccharide impurity relative to the total weight of the cell-surface polysaccharide enriched from C. difficile.

Embodiment 224. The pharmaceutical composition of any one of embodiments 173-215, wherein the pharmaceutical composition comprises less than about 5% by weight of a mixture of impurities relative to the total weight of the cell-surface polysaccharide enriched from C. difficile, wherein the mixture of impurities is selected from the group consisting of
(a) a mixture of nucleic acid and protein impurities relative to the total weight of the cell-surface polysaccharide enriched from C. difficile;
(b) a mixture of nucleic acid and peptidoglycan impurities relative to the total weight of the cell-surface polysaccharide enriched from C. difficile;
(c) a mixture of peptidoglycan and protein impurities relative to the total weight of the cell-surface polysaccharide enriched from C. difficile;
(d) a mixture of saccharide and protein impurities relative to the total weight of the cell-surface polysaccharide enriched from C. difficile;
(e) a mixture of peptidoglycan and saccharide impurities; and
(f) a mixture of nucleic acid and saccharide impurities relative to the total weight of the PSII antigen.

Embodiment 225. The pharmaceutical composition of any one of embodiments 173-215, wherein the pharmaceutical composition comprises less than about 5% by weight of a mixture of impurities relative to the total weight of the cell-surface polysaccharide enriched from C. difficile, wherein the mixture of impurities is selected from the group consisting of
(a) a mixture of nucleic acid, protein and peptidoglycan impurities relative to the total weight of the cell-surface polysaccharide enriched from C. difficile;
(b) a mixture of saccharide, protein and peptidoglycan impurities relative to the total weight of the cell-surface polysaccharide enriched from C. difficile;
(c) a mixture of nucleic acid, protein and saccharide impurities relative to the total weight of the cell-surface polysaccharide enriched from C. difficile; and
(d) a mixture of nucleic acid, peptidoglycan and saccharide impurities relative to the total weight of the cell-surface polysaccharide enriched from C. difficile.

Embodiment 226. The pharmaceutical composition of any one of embodiments 173-225, comprising less than about 5% by weight of a mixture of nucleic acid, peptidoglycan, saccharide and protein impurities relative to the total weight of the cell-surface polysaccharide enriched from C. difficile.

Embodiment 227. The pharmaceutical composition of any one of embodiments 173-226, comprising at least 5% by weight of the cell-surface polysaccharide enriched from C. difficile.

Embodiment 228. A pharmaceutical composition comprising:
(a) a cell-surface polysaccharide enriched from Clostridioides difficile (C. difficile);
(b) a first polypeptide comprising a first toxoid of C. difficile or a fragment thereof; a
(c) a second polypeptide comprising a second toxoid of C. difficile or a fragment thereof; and
(d) a pharmaceutically acceptable carrier;
   wherein the cell-surface polysaccharide enriched from C. difficile is not conjugated to the first polypeptide or the second polypeptide.

Embodiment 229. The pharmaceutical composition of embodiment 228, wherein the first polypeptide is a toxoid of C. difficile toxin A (TcdA) or a fragment thereof.

Embodiment 230. The pharmaceutical composition of embodiment 228 or 229, wherein the first polypeptide is a full-length toxoid of C. difficile toxin A.

Embodiment 231. The pharmaceutical composition of any one of embodiments 228-230, wherein the second polypeptide is a toxoid of C. difficile toxin B (TcdB) or a fragment thereof.

Embodiment 232. The pharmaceutical composition of any one of embodiments 228-231, wherein the second polypeptide is a full-length toxoid of toxin B.

Embodiment 233. The pharmaceutical composition of any one of embodiments 228-232, wherein the cell-surface polysaccharide from C. difficile is an anionic cell-surface polysaccharide.

Embodiment 234. The pharmaceutical composition of any one of embodiments 228-233, wherein the cell-surface polysaccharide from C. difficile is unsubstituted or substituted.

Embodiment 235. The pharmaceutical composition any one of embodiments 228-234, wherein the first polypeptide and the second polypeptide are fused.

Embodiment 236. The pharmaceutical composition of any one of embodiments 228-235, wherein the first polypeptide has at least 80% sequence identity to any one of SEQ ID NO: 4-6.

Embodiment 237. The pharmaceutical composition of any one of embodiments 228-236, wherein the second polypeptide has at least 80% sequence identity to SEQ ID NO: 1-3 or 7-40.

Embodiment 238. The pharmaceutical composition of any one of embodiments 228-237, wherein a ratio of the cell-surface polysaccharide from C. difficile to the first polypeptide is from about 10:1 to about 1:10.

Embodiment 239. The pharmaceutical composition of any one of embodiments 228-238, wherein a ratio of the cell-surface polysaccharide from C. difficile to the second polypeptide is about 10:1 to about 1:10.

Embodiment 240. The pharmaceutical composition of any one of embodiments 228-239, wherein the cell-surface polysaccharide enriched from C. difficile is PSII, a pharmaceutically acceptable salt, or an immunogenic fragment thereof.

Embodiment 241. The pharmaceutical composition of embodiment 240, wherein the PSII, the pharmaceutically acceptable salt, or the immunogenic fragment thereof comprises a phosphate moiety.

Embodiment 242. The pharmaceutical composition of any one of embodiments 228-241, wherein the PSII is a polysaccharide of Formula (I):

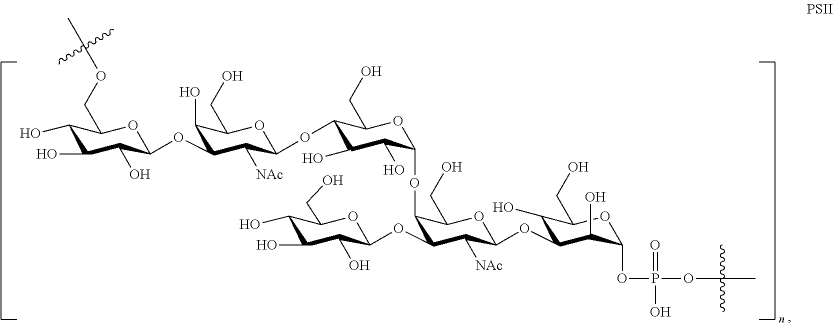

wherein n is an integer of from 1 to 100.

Embodiment 243. The pharmaceutical composition of any one of embodiments 228-242, wherein the cell-surface polysaccharide has a molecular weight of from about 5 kDa to about 10 kDa.

Embodiment 244. The pharmaceutical composition of any one of embodiments 228-243, wherein the cell-surface polysaccharide has a molecular weight of about 8.8 kDa.

Embodiment 245. The pharmaceutical composition of any one of embodiments 228-244, further comprising an adjuvant.

Embodiment 246. The pharmaceutical composition of embodiment 245, wherein the adjuvant comprises an aluminum-based adjuvant.

Embodiment 247. The pharmaceutical composition of embodiment 245, wherein the adjuvant comprises aluminum hydroxide.

Embodiment 248. The pharmaceutical composition of embodiment 245, wherein the adjuvant comprises aluminum phosphate.

Embodiment 249. The pharmaceutical composition of embodiment 245, wherein the adjuvant comprises delta inulin microparticles.

Embodiment 250. The pharmaceutical composition of any one of embodiments 228-249, wherein the cell-surface polysaccharide enriched from C. difficile is conjugated to a pharmaceutically acceptable carrier.

Embodiment 251. The pharmaceutical composition of embodiment 250, wherein the pharmaceutically acceptable carrier is a mutant of a diphtheria toxin.

Embodiment 252. The pharmaceutical composition of embodiment 250, wherein the pharmaceutically acceptable carrier is CRM₁₉₇.

Embodiment 253. The pharmaceutical composition of any one of embodiments 250-252, wherein the cell-surface polysaccharide enriched from C. difficile is conjugated to the pharmaceutically acceptable carrier by a chemical linker.

Embodiment 254. The pharmaceutical composition of embodiment 253, wherein the chemical linker comprises a thiosuccinimide.

Embodiment 255. The pharmaceutical composition of embodiment 253, wherein the chemical linker comprises a thioester.

Embodiment 256. The pharmaceutical composition of any one of embodiments 228-255, wherein the cell-surface polysaccharide enriched from C. difficile is conjugated to CRM₁₉₇ and has a molecular weight of from about 100 kDa to about 1000 kDa.

Embodiment 257. The pharmaceutical composition of any one of embodiments 228-256, wherein the cell-surface polysaccharide enriched from C. difficile is conjugated to CRM₁₉₇ and has a molecular weight of from about 100 kDa to about 350 kDa.

Embodiment 258. The pharmaceutical composition of any one of embodiments 228-257, wherein the cell-surface polysaccharide enriched from C. difficile is a native cell-surface polysaccharide from C. difficile.

Embodiment 259. The pharmaceutical composition of any one of embodiments 228-258, wherein the cell-surface polysaccharide enriched from C. difficile is non-synthetic.

Embodiment 260. The pharmaceutical composition of any one of embodiments 228-259, wherein the cell-surface polysaccharide enriched from C. difficile is purified from one or more strains of C. difficile.

Embodiment 261. The pharmaceutical composition of any one of embodiments 228-260, wherein the cell-surface polysaccharide enriched from C. difficile is from a cell-surface extract (CSE) of one or more strains of C. difficile.

Embodiment 262. The pharmaceutical composition of any one of embodiments 228-261, wherein the pharmaceutical composition comprises less than about 20% by weight of a polypeptide from C. difficile.

Embodiment 263. The pharmaceutical composition of any one of embodiments 228-262, wherein the C. difficile is ribotype 001, 003, 012, 014, 027, 036, 106, MOH 900, or MOH 718.

Embodiment 264. The pharmaceutical composition of any one of embodiments 228-263, wherein the pharmaceutical composition is an immunogenic pharmaceutical composition or a vaccine.

Embodiment 265. The pharmaceutical composition of embodiment 264, wherein the pharmaceutical composition induces neutralization titers against the cell-surface polysaccharide enriched from C. difficile.

Embodiment 266. The pharmaceutical composition of embodiment 264 or 265, wherein the pharmaceutical composition induces neutralization titers against C. difficile toxin A or C. difficile toxin B.

Embodiment 267. The pharmaceutical composition of embodiment 264 or 265, wherein the pharmaceutical composition induces neutralization titers against C. difficile toxin A and C. difficile toxin B.

Embodiment 268. The pharmaceutical composition of any one of embodiments 228-267, wherein the pharmaceutical composition comprises less than about 5% by weight of an impurity relative to the total weight of the cell-surface polysaccharide enriched from C. difficile.

Embodiment 269. The pharmaceutical composition of embodiment 268, wherein the impurity is a peptidoglycan, a protein, a nucleic acid, a saccharide, or a combination thereof.

Embodiment 270. The pharmaceutical composition of embodiment 268 or 269, wherein the impurity is a C. difficile impurity.

Embodiment 271. The pharmaceutical composition of any one of embodiments 268-270, wherein the impurity is a nucleic acid.

Embodiment 272. The pharmaceutical composition of any one of embodiments 268-270, wherein the impurity is a saccharide.

Embodiment 273. The pharmaceutical composition of any one of embodiments 268-270, wherein the impurity is derived from a cell-surface extract of C. difficile.

Embodiment 274. The pharmaceutical composition of any one of embodiments 228-273, wherein the pharmaceutical composition comprises less than about 5% by weight of a peptidoglycan impurity relative to the total weight of the cell-surface polysaccharide enriched from C. difficile.

Embodiment 275. The pharmaceutical composition of any one of embodiments 228-274, wherein the pharmaceutical composition comprises less than about 5% by weight of a protein impurity relative to the total weight of the cell-surface polysaccharide enriched from C. difficile.

Embodiment 276. The pharmaceutical composition of any one of embodiments 228-275, wherein the pharmaceutical composition comprises less than about 5% by weight of a nucleic acid impurity relative to the total weight of the cell-surface polysaccharide enriched from C. difficile.

Embodiment 277. The pharmaceutical composition of any one of embodiments 228-276, wherein the pharmaceutical composition comprises less than about 5% by weight of a saccharide impurity relative to the total weight of the cell-surface polysaccharide enriched from C. difficile.

Embodiment 278. The pharmaceutical composition of any one of embodiments 228-268, wherein the pharmaceutical composition comprises less than about 5% by weight of a mixture of impurities relative to the total weight of the cell-surface polysaccharide enriched from C. difficile, wherein the mixture of impurities is selected from the group consisting of
(a) a mixture of nucleic acid and protein impurities relative to the total weight of the cell-surface polysaccharide enriched from C. difficile;
(b) a mixture of nucleic acid and peptidoglycan impurities relative to the total weight of the cell-surface polysaccharide enriched from C. difficile;
(c) a mixture of peptidoglycan and protein impurities relative to the total weight of the cell-surface polysaccharide enriched from C. difficile;
(d) a mixture of saccharide and protein impurities relative to the total weight of the cell-surface polysaccharide enriched from C. difficile;
(e) a mixture of peptidoglycan and saccharide impurities; and
(f) a mixture of nucleic acid and saccharide impurities relative to the total weight of the PSII antigen.

Embodiment 279. The pharmaceutical composition of any one of embodiments 228-268, wherein the pharmaceutical composition comprises less than about 5% by weight of a mixture of impurities relative to the total weight of the cell-surface polysaccharide enriched from C. difficile, wherein the mixture of impurities is selected from the group consisting of
(a) a mixture of nucleic acid, protein and peptidoglycan impurities relative to the total weight of the cell-surface polysaccharide enriched from C. difficile;
(b) a mixture of saccharide, protein and peptidoglycan impurities relative to the total weight of the cell-surface polysaccharide enriched from C. difficile;
(c) a mixture of nucleic acid, protein and saccharide impurities relative to the total weight of the cell-surface polysaccharide enriched from C. difficile; and
(d) a mixture of nucleic acid, peptidoglycan and saccharide impurities relative to the total weight of the cell-surface polysaccharide enriched from C. difficile.

Embodiment 280. The pharmaceutical composition of any one of embodiments 228-279, comprising less than about 5% by weight of a mixture of nucleic acid, peptidoglycan, saccharide and protein impurities relative to the total weight of the cell-surface polysaccharide enriched from C. difficile.

Embodiment 281. The pharmaceutical composition of any one of embodiments 228-280, comprising at least 5% by weight of the cell-surface polysaccharide enriched from C.

Embodiment 282. A pharmaceutical composition consisting essentially of:
(a) a cell-surface polysaccharide from Clostridioides difficile (C. difficile) conjugated to a pharmaceutically acceptable carrier,
(b) a first polypeptide or a first polynucleotide encoding the first polypeptide, wherein the first polypeptide comprises a first toxoid of C. difficile or a fragment thereof,
(c) a second polypeptide or a second polynucleotide encoding the second polypeptide, wherein the second polypeptide comprises a second toxoid of C. difficile or a fragment thereof; and
(d) an adjuvant.

Embodiment 283. The pharmaceutical composition of embodiment 282, wherein the first polypeptide is a toxoid of C. difficile toxin A (TcdA) or a fragment thereof.

Embodiment 284. The pharmaceutical composition of embodiment 282 or 283, wherein the first polypeptide is a full-length toxoid of C. difficile toxin A.

Embodiment 285. The pharmaceutical composition of any one of embodiments 282-284, wherein the second polypeptide is a toxoid of C. difficile toxin B (TcdB) or a fragment thereof.

Embodiment 286. The pharmaceutical composition of any one of embodiments 282-285, wherein the second polypeptide is a full-length toxoid of toxin B.

Embodiment 287. The pharmaceutical composition of any one of embodiments 282-286, wherein the cell-surface polysaccharide from C. difficile is an anionic cell-surface polysaccharide.

Embodiment 288. The pharmaceutical composition of any one of embodiments 282-287, wherein the cell-surface polysaccharide from C. difficile is unsubstituted or substituted.

Embodiment 289. The pharmaceutical composition any one of embodiments 282-288, wherein the first polypeptide and the second polypeptide are fused.

Embodiment 290. The pharmaceutical composition of any one of embodiments 282-289, wherein the first polypeptide has at least 80% sequence identity to any one of SEQ ID NO: 4-6.

Embodiment 291. The pharmaceutical composition of any one of embodiments 282-290, wherein the second polypeptide has at least 80% sequence identity to SEQ ID NO: 1-3 or 7-40.

Embodiment 292. The pharmaceutical composition of any one of embodiments 282-291, wherein a ratio of the cell-surface polysaccharide from C. difficile to the first polypeptide is from about 10:1 to about 1:10.

Embodiment 293. The pharmaceutical composition of any one of embodiments 282-292, wherein a ratio of the cell-surface polysaccharide from C. difficile to the second polypeptide is about 10:1 to about 1:10.

Embodiment 294. The pharmaceutical composition of any one of embodiments 282-293, wherein the cell-surface polysaccharide enriched from C. difficile is PSII, a pharmaceutically acceptable salt, or an immunogenic fragment thereof.

Embodiment 295. The pharmaceutical composition of embodiment 294, wherein the PSII, the pharmaceutically acceptable salt, or the immunogenic fragment thereof comprises a phosphate moiety.

Embodiment 296. The pharmaceutical composition of embodiment 295, wherein the PSII is a polysaccharide of Formula (I):

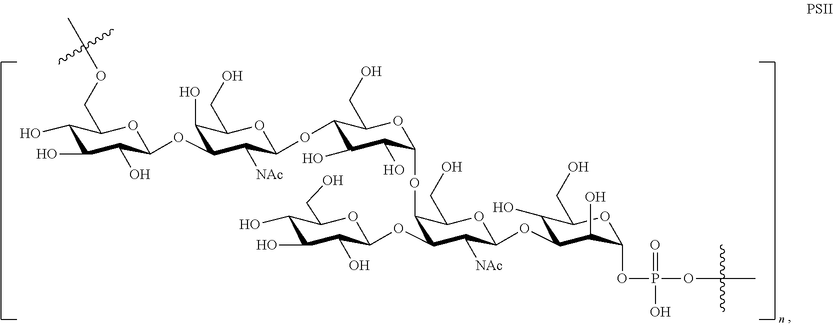

wherein n is an integer of from 1 to 100.

Embodiment 297. The pharmaceutical composition of any one of embodiments 282-296, wherein the cell-surface polysaccharide has a molecular weight of from about 5 kDa to about 10 kDa.

Embodiment 298. The pharmaceutical composition of any one of embodiments 282-297, wherein the cell-surface polysaccharide has a molecular weight of about 8.8 kDa.

Embodiment 299. The pharmaceutical composition of any one of embodiments 282-298, wherein the adjuvant comprises an aluminum-based adjuvant.

Embodiment 300. The pharmaceutical composition of any one of embodiments 282-298, wherein the adjuvant comprises aluminum hydroxide.

Embodiment 301. The pharmaceutical composition of any one of embodiments 282-298, wherein the adjuvant comprises aluminum phosphate.

Embodiment 302. The pharmaceutical composition of any one of embodiments 282-298, wherein the adjuvant comprises delta inulin microparticles.

Embodiment 303. The pharmaceutical composition of any one of embodiments 282-302, wherein the pharmaceutically acceptable carrier is a mutant of a diphtheria toxin.

Embodiment 304. The pharmaceutical composition of any one of embodiments 282-302, wherein the pharmaceutically acceptable carrier is CRM₁₉₇.

Embodiment 305. The pharmaceutical composition of any one of embodiments 282-304, wherein the cell-surface polysaccharide enriched from C. difficile is conjugated to the pharmaceutically acceptable carrier by a chemical linker.

Embodiment 306. The pharmaceutical composition of embodiment 305, wherein the chemical linker comprises a thiosuccinimide.

Embodiment 307. The pharmaceutical composition of embodiment 305, wherein the chemical linker comprises a thioester.

Embodiment 308. The pharmaceutical composition of any one of embodiments 282-307, wherein the cell-surface polysaccharide enriched from C. difficile is conjugated to CRM₁₉₇ and has a molecular weight of from about 100 kDa to about 1000 kDa.

Embodiment 309. The pharmaceutical composition of any one of embodiments 282-308, wherein the cell-surface polysaccharide enriched from C. difficile is conjugated to CRM₁₉₇ and has a molecular weight of from about 100 kDa to about 350 kDa.

Embodiment 310. The pharmaceutical composition of any one of embodiments 282-309, wherein the cell-surface polysaccharide enriched from C. difficile is a native cell-surface polysaccharide from C. difficile.

Embodiment 311. The pharmaceutical composition of any one of embodiments 282-310, wherein the cell-surface polysaccharide enriched from C. difficile is non-synthetic.

Embodiment 312. The pharmaceutical composition of any one of embodiments 282-311, wherein the cell-surface polysaccharide enriched from C. difficile is purified from one or more strains of C. difficile.

Embodiment 313. The pharmaceutical composition of any one of embodiments 282-312, wherein the cell-surface polysaccharide enriched from C. difficile is from a cell-surface extract (CSE) of one or more strains of C. difficile.

Embodiment 314. The pharmaceutical composition of any one of embodiments 282-313, wherein the pharmaceutical composition comprises less than about 20% by weight of a polypeptide from C. difficile.

Embodiment 315. The pharmaceutical composition of any one of embodiments 282-314, wherein the C. difficile is ribotype 001, 003, 012, 014, 027, 036, 106, MOH 900, or MOH 718.

Embodiment 316. The pharmaceutical composition of any one of embodiments 282-315, wherein the pharmaceutical composition is an immunogenic pharmaceutical composition or a vaccine.

Embodiment 317. The pharmaceutical composition of embodiment 316, wherein the pharmaceutical composition induces neutralization titers against the cell-surface polysaccharide enriched from C. difficile.

Embodiment 318. The pharmaceutical composition of embodiment 316 or 317, wherein the pharmaceutical composition induces neutralization titers against C. difficile toxin A or C. difficile toxin B.

Embodiment 319. The pharmaceutical composition of embodiment 316 or 317, wherein the pharmaceutical composition induces neutralization titers against C. difficile toxin A and C. difficile toxin B.

Embodiment 320. The pharmaceutical composition of any one of embodiments 228-319, wherein the pharmaceutical composition comprises less than about 5% by weight of an impurity relative to the total weight of the cell-surface polysaccharide enriched from C. difficile.

Embodiment 321. The pharmaceutical composition of embodiment 320, wherein the impurity is a peptidoglycan, a protein, a nucleic acid, a saccharide, or a combination thereof.

Embodiment 322. The pharmaceutical composition of embodiment 320 or 321, wherein the impurity is a C. difficile impurity.

Embodiment 323. The pharmaceutical composition of any one of embodiments 320-322, wherein the impurity is a nucleic acid.

Embodiment 324. The pharmaceutical composition of any one of embodiments 320-322, wherein the impurity is a saccharide.

Embodiment 325. The pharmaceutical composition of any one of embodiments 320-322, wherein the impurity is derived from a cell-surface extract of C. difficile.

Embodiment 326. The pharmaceutical composition of any one of embodiments 282-325, wherein the pharmaceutical composition comprises less than about 5% by weight of a peptidoglycan impurity relative to the total weight of the cell-surface polysaccharide enriched from C. difficile.

Embodiment 327. The pharmaceutical composition of any one of embodiments 282-326, wherein the pharmaceutical composition comprises less than about 5% by weight of a protein impurity relative to the total weight of the cell-surface polysaccharide enriched from C. difficile.

Embodiment 328. The pharmaceutical composition of any one of embodiments 282-327, wherein the pharmaceutical composition comprises less than about 5% by weight of a nucleic acid impurity relative to the total weight of the cell-surface polysaccharide enriched from C. difficile.

Embodiment 329. The pharmaceutical composition of any one of embodiments 282-328, wherein the pharmaceutical composition comprises less than about 5% by weight of a saccharide impurity relative to the total weight of the cell-surface polysaccharide enriched from C. difficile.

Embodiment 330. The pharmaceutical composition of any one of embodiments 282-320, wherein the pharmaceutical composition comprises less than about 5% by weight of a mixture of impurities relative to the total weight of the cell-surface polysaccharide enriched from C. difficile, wherein the mixture of impurities is selected from the group consisting of
(a) a mixture of nucleic acid and protein impurities relative to the total weight of the cell-surface polysaccharide enriched from C. difficile;
(b) a mixture of nucleic acid and peptidoglycan impurities relative to the total weight of the cell-surface polysaccharide enriched from C. difficile;
(c) a mixture of peptidoglycan and protein impurities relative to the total weight of the cell-surface polysaccharide enriched from C. difficile;
(d) a mixture of saccharide and protein impurities relative to the total weight of the cell-surface polysaccharide enriched from C. difficile;
(e) a mixture of peptidoglycan and saccharide impurities; and
(f) a mixture of nucleic acid and saccharide impurities relative to the total weight of the PSII antigen.

Embodiment 331. The pharmaceutical composition of any one of embodiments 282-320, wherein the pharmaceutical composition comprises less than about 5% by weight of a mixture of impurities relative to the total weight of the cell-surface polysaccharide enriched from C. difficile, wherein the mixture of impurities is selected from the group consisting of
(a) a mixture of nucleic acid, protein and peptidoglycan impurities relative to the total weight of the cell-surface polysaccharide enriched from C. difficile;
(b) a mixture of saccharide, protein and peptidoglycan impurities relative to the total weight of the cell-surface polysaccharide enriched from C. difficile;
(c) a mixture of nucleic acid, protein and saccharide impurities relative to the total weight of the cell-surface polysaccharide enriched from C. difficile; and
(d) a mixture of nucleic acid, peptidoglycan and saccharide impurities relative to the total weight of the cell-surface polysaccharide enriched from C. difficile.

Embodiment 332. The pharmaceutical composition of any one of embodiments 282-331, comprising less than about 5% by weight of a mixture of nucleic acid, peptidoglycan, saccharide and protein impurities relative to the total weight of the cell-surface polysaccharide enriched from C. difficile.

Embodiment 333. The pharmaceutical composition of any one of embodiments 282-332, comprising at least 5% by weight of the cell-surface polysaccharide enriched from C. difficile.

Embodiment 334. A method of treating an infection comprising:
(a) administering a therapeutically effective amount of a composition of any one of embodiments 1-171 or a pharmaceutical composition of any one of embodiments 172-333 to a subject in need thereof; wherein the infection is a Clostridioides Difficile (C. difficile) infection;
(b) after the administering of the pharmaceutical composition, collecting a fecal sample from the subject; and
(c) analyzing the fecal sample and determining a change in colony-forming units (CFU)/mg of C. difficile in feces a marker of infection.

Embodiment 335. The method of embodiment 334, wherein the subject does not exhibit symptoms of the C. difficile infection.

Embodiment 336. The method of embodiment 334 or 335, wherein the administering is parenteral.

Embodiment 337. The method of embodiment 334 or 335, wherein the administering is intravenous.

Embodiment 338. The method of embodiment 334 or 335, wherein the administering is intramuscular.

Embodiment 339. The method of any one of embodiments 334-338, wherein the collecting of the fecal sample is performed at least 1 day after the administering of the composition or the pharmaceutical composition.

Embodiment 340. The method of embodiment 339, wherein the collecting of the fecal sample is performed at least 6 days after the administering of the composition or the pharmaceutical composition.

Embodiment 341. The method of embodiment 339, wherein the collecting of the fecal sample is performed at least 9 days after the administering of the composition or the pharmaceutical composition.

Embodiment 342. The method of embodiment 339, wherein the collecting of the fecal sample is performed at least 14 days after the administering of the composition or the pharmaceutical composition.

Embodiment 343. The method of any one of embodiments 334-342, wherein the analyzing comprises plating and qPCR analysis.

Embodiment 344. The method of any one of embodiments 334-343, wherein the method comprises measuring a percent reduction in CFU/mg feces of C. difficile in feces, and adjusting dosage/treatment.

Embodiment 345. The method of embodiment 344, wherein the percent reduction is at least a 60% reduction of CFU/mg of C. difficile compared to an absence of the therapeutically effective amount of the pharmaceutical composition.

Embodiment 346. The method of any one of embodiments 334-345, wherein if the fecal sample has an increase in CFU/mg compared to a fecal sample in the absence of administering of the pharmaceutical composition, administering a second therapeutically effective amount of the pharmaceutical composition, wherein the second therapeutically effective amount is greater than the therapeutically effective amount of the pharmaceutical composition.

Embodiment 347. The method of embodiment 334, wherein if the fecal sample has a decrease in CFU/mg compared to a fecal sample in the absence of administering of the pharmaceutical composition, administering a third therapeutically effective amount of the pharmaceutical composition, wherein the third therapeutically effective amount is less than the therapeutically effective amount of the pharmaceutical composition.

Embodiment 348. The method of any one of embodiments 334-347, wherein the administering is weekly.

Embodiment 349. The method of any one of embodiments 334-347, wherein the administering is weekly for two weeks.

Embodiment 350. The method of any one of embodiments 334-347, wherein the administering is biweekly.

Embodiment 351. The method of any one of embodiments 334-347, wherein the administering is biweekly for one month.

Embodiment 352. The method of any one of embodiments 334-351, wherein the subject is a resident of a nursing home.

Embodiment 353. The method of any one of embodiments 334-352, wherein the subject has a history of a C. difficile infection.

Embodiment 354. The method of any one of embodiments 334-353, wherein the subject has a positive C. difficile CFU/mg count.

Embodiment 355. The method of any one of embodiments 334-354, further comprising a therapeutic agent.

Embodiment 356. The method of embodiment 355, wherein the therapeutic agent is an antibiotic.

Embodiment 357. The method of embodiment 356, wherein the antibiotic is vancomycin.

Embodiment 358. The method of embodiment 356, wherein the antibiotic is fidaxomicin.

Embodiment 359. A method of treating an infection, the method comprising administering to a subject in need thereof a therapeutically effective amount of a pharmaceutical composition comprising
(a) a cell-surface polysaccharide of Clostridioides Difficile (C. difficile); and
(b) a polypeptide or polynucleotide encoding the polypeptide, wherein the polypeptide comprises a toxoid of C. difficile or a fragment thereof;
wherein the administering is weekly or biweekly, and
wherein the infection is caused by C. difficile.

Embodiment 360. The method of embodiment 359, wherein the pharmaceutical composition is a pharmaceutical composition of any one of embodiments 172-333.

Embodiment 361. The method of embodiment 359 or 360, wherein the subject does not exhibit symptoms of the C. difficile infection.

Embodiment 362. The method of any one of embodiments 359-361, wherein the administering is parenteral.

Embodiment 363. The method of any one of embodiments 359-361, wherein the administering is intravenous.

Embodiment 364. The method of any one of embodiments 359-361, wherein the administering is intramuscular.

Embodiment 365. The method of any one of embodiments 359334-364, wherein the method comprises measuring a percent reduction in CFU/mg feces of C. difficile in feces, and adjusting dosage/treatment.

Embodiment 366. The method of any one of embodiments 365, wherein the percent reduction is at least a 60% reduction of CFU/mg of C. difficile compared to an absence of the therapeutically effective amount of the pharmaceutical composition.

Embodiment 367. The method of any one of embodiments 359-366, wherein if a fecal sample obtained from the subject has an increase in CFU/mg compared to a fecal sample in the absence of administering of the pharmaceutical composition, administering a second therapeutically effective amount of the pharmaceutical composition, wherein the second therapeutically effective amount is greater than the therapeutically effective amount of the pharmaceutical composition.

Embodiment 368. The method of embodiment 367, wherein if the fecal sample has a decrease in CFU/mg compared to a fecal sample in the absence of administering of the pharmaceutical composition, administering a third therapeutically effective amount of the pharmaceutical composition, wherein the third therapeutically effective amount is less than the therapeutically effective amount of the pharmaceutical composition.

Embodiment 369. The method of any one of embodiments 359-368, wherein the administering is weekly for two weeks.

Embodiment 370. The method of any one of embodiments 359-368, wherein the administering is biweekly.

Embodiment 371. The method of any one of embodiments 359-368, wherein the administering is biweekly for one month.

Embodiment 372. The method of any one of embodiments 359-371, wherein the subject is a resident of a nursing home.

Embodiment 373. The method of any one of embodiments 359-372, wherein the subject has a history of a C. difficile infection.

Embodiment 374. The method of any one of embodiments 359-373, wherein the subject has a positive C. difficile CFU/mg count.

Embodiment 375. The method of any one of embodiments 359-374, further comprising a therapeutic agent.

Embodiment 376. The method of embodiment 375, wherein the therapeutic agent is an antibiotic.

Embodiment 377. The method of embodiment 375, wherein the antibiotic is vancomycin.

Embodiment 378. The method of embodiment 375, wherein the antibiotic is fidaxomicin.

Embodiment 379. A method comprising:
(a) selecting a subject from a group consisting of:
   (i) a first subject, wherein the first subject is greater than 55 years of age;
   (ii) a second subject, wherein the second has a history of a C. difficile infection within a 6-month period; and
   (iii) a third subject, wherein the third subject has a positive C. difficile colony forming units (CFU)/mg count; and
(b) administering a therapeutically effective amount of a pharmaceutical composition comprising a cell-surface polysaccharide of C. difficile; and a polypeptide or polynucleotide encoding the polypeptide, wherein the polypeptide comprises a toxoid of C. difficile or a fragment thereof.

Embodiment 380. The method of embodiment 379, wherein the pharmaceutical composition is a pharmaceutical composition of any one of embodiments 172-333.

Embodiment 381. The method of embodiment 379, wherein the first subject is a resident of a nursing home.

Embodiment 382. The method of embodiment 379, wherein the first subject has a history of a C. difficile infection.

Embodiment 383. The method of embodiment 379, wherein the first subject has a treatment history with a broad spectrum antibiotic.

Embodiment 384. The method of embodiment 379, wherein the second subject has a positive C. difficile CFU/mg count.

Embodiment 385. A method of treating an infection,
(a) identifying genetic sequence information of a biological sample obtained from a subject to determine a presence of the infection, wherein the infection is a Clostridioides difficile (C. difficile) infection; and
(b) administering to the subject a therapeutically effective amount of a pharmaceutical composition comprising a cell-surface polysaccharide of C. difficile.

Embodiment 386. The method of embodiment 385, wherein the pharmaceutical composition is a pharmaceutical composition of any one of embodiments 172-333.

Embodiment 387. The method of embodiment 385, wherein the method comprises performing an assay, wherein the assay is a real-time polymerase chain reaction (RT-PCR) or nucleic acid amplification test (NAAT).

Embodiment 388. The method of embodiment 385, wherein the assay is performed on a fecal sample from the subject.

Embodiment 389. The method of embodiment 388, wherein the assay determines CFU/mg of C. difficile in the fecal sample.

Embodiment 390. A method of enriching for a cell-surface polysaccharide of Clostridioides difficile (C. difficile) comprising:
(a) obtaining a cell-surface extract (CSE) of one or more strains of C. difficile, and
(b) enriching for a cell-surface polysaccharide of C. difficile from the CSE, thereby obtaining an enriched cell-surface polysaccharide of C. difficile sample;
wherein the enriched cell-surface polysaccharide of C. difficile sample comprises less than about 5% by weight of a C. difficile impurity.

Embodiment 391. The method of embodiment 390, wherein the method comprises: enriching for PSII from the CSE, thereby obtaining an enriched PSII sample;
(a) wherein the enriched PSII sample comprises PSII and (a) a level of peptidoglycan contamination that is less than 5% by weight peptidoglycan relative to a total weight of the PSII;
(b) a level of protein contamination that is less than 5% by weight protein relative to the total weight of the PSII; or
(c) a level of nucleic acid contamination that is less than 5% by weight nucleic acid relative to the total weight of the PSII.

Embodiment 392. The method of embodiment 390, wherein the obtaining comprises stripping one or more strains of C. difficile.

Embodiment 393. The method of embodiment 392, wherein the one or more strains of C. difficile is ribotype 001, 003, 012, 014, 027, 036, 106, MOH 900, or MOH 718.

Embodiment 394. The method of any one of embodiments 390-393, wherein the enriched cell-surface polysaccharide of C. difficile sample comprises less than about 5% by weight of a C. difficile protein.

Embodiment 395. The method of any one of embodiments 390-394, wherein the cell-surface polysaccharide of C. difficile is selected from the group consisting of PSI, PSII, PSIII, pharmaceutically acceptable salts thereof, and immunogenic fragments thereof.

Embodiment 396. The method of any one of embodiments 390-395, wherein the cell-surface polysaccharide of C. difficile comprises a phosphate moiety.

Embodiment 397. The method of any one of embodiments 390-396, wherein the cell-surface polysaccharide of C. difficile is PSII.

Embodiment 398. The method of any one of embodiments 390-397, wherein the enriching comprises a step of ethanol precipitation.

Embodiment 399. The method of any one of embodiments 390-398, wherein the enriching comprises one or more steps of TCA precipitation.

Embodiment 400. The method of any one of embodiments 390-399, wherein the enriching comprises a step of ultrafiltration/diafiltration (UFDF).

Embodiment 401. The method of any one of embodiments 390-400, wherein the enriching comprises a step of ion exchange chromatography.

Embodiment 402. The method of embodiment 401, wherein the enriching comprises one or more steps of TCA precipitation after the step of ion exchange chromatography.

Embodiment 403. The method of embodiment 401 or 402, wherein the enriching comprises one or more steps of ultrafiltration/diafiltration (UFDF) after the step of TCA precipitation and/or after the step of ion exchange chromatography.

Embodiment 404. The method of any one of embodiments 401-403, wherein the enriching comprises a step of filtration.

Embodiment 405. The method of embodiment 404, wherein the step of filtration comprises tangential flow filtration or centrifugation through a filter with a molecular weight cut off.

Embodiment 406. The method of embodiment 405, wherein the filter has a molecular weight cut off of 3 kDa or less.

Embodiment 407. The method of embodiment 405, wherein the filter has a molecular weight cut off of 10 kDa or more.

Embodiment 408. The method of any one of embodiments 390-407, wherein the method further comprises lyophilization.

Embodiment 409. The method of any one of embodiments 391-407, wherein the enriched cell-surface polysaccharide of C. difficile sample comprises PSII and a level of peptidoglycan contamination that is less than 5% by weight peptidoglycan relative to the total weight of the PSII according to NMR.

Embodiment 410. The method of any one of embodiments 391-409, wherein the enriched cell-surface polysaccharide of C. difficile sample comprises PSII and a level of protein contamination that is less than 5% by weight protein relative to the total weight of the PSII according to NMR.

Embodiment 411. The method of any one of embodiments 391-410, wherein the enriched cell-surface polysaccharide of C. difficile sample comprises PSII and a level of nucleic acid contamination that is less than 5% by weight nucleic acid relative to the total weight of the PSII according to NMR.

1. A pharmaceutical composition comprising:
(a) a cell-surface polysaccharide of Clostridioides difficile (C. difficile); and
(b) a first polypeptide comprising a carrier protein derived from an organism other than C. difficile;
wherein the carrier protein and the cell-surface polysaccharide are present in the composition at a ratio of from about 10:1 to about 1:10.

2. The pharmaceutical composition of claim 1, further comprising a second polypeptide or a first polynucleotide encoding the second polypeptide, wherein the second polypeptide is a toxoid of C. difficile toxin A (TcdA) or a fragment thereof.

3. (canceled)

4. (canceled)

5. The pharmaceutical composition of claim 2, further comprising a third polypeptide or a second polynucleotide encoding the third polypeptide, wherein the third polypeptide is a toxoid of C. difficile toxin B (TcdB) or a fragment thereof.

6. (canceled)

7. (canceled)

8. The pharmaceutical composition of claim 1, wherein the cell-surface polysaccharide from C. difficile is an anionic cell-surface polysaccharide.

9. (canceled)

10. The pharmaceutical composition of claim 5, wherein the second polypeptide and the third polypeptide are fused.

11. The pharmaceutical composition of claim 2, wherein the second polypeptide has at least 80% sequence identity to any one of SEQ ID NO: 4-6.

12. The pharmaceutical composition of claim 5, wherein the third polypeptide has at least 80% sequence identity to SEQ ID NO: 1-3 or 7-40.

13. The pharmaceutical composition of claim 2, wherein a ratio of the cell-surface polysaccharide from C. difficile to the second polypeptide is from about 10:1 to about 1:10.

14. The pharmaceutical composition of claim 5, wherein a ratio of the cell-surface polysaccharide from C. difficile to the third polypeptide is about 10:1 to about 1:10.

15. The pharmaceutical composition of claim 1, wherein the cell-surface polysaccharide from C. difficile is PSII, a pharmaceutically acceptable salt, or an immunogenic fragment thereof.

16. (canceled)

17. The pharmaceutical composition of claim 5, wherein the cell-surface polysaccharide is not conjugated to the second polypeptide or the third polypeptide.

18. The pharmaceutical composition of claim 1, wherein the cell-surface polysaccharide from C. difficile is from a cell-surface extract (CSE) of one or more strains of C. difficile, wherein the one or more strains of C. difficile is a C. difficile ribotype selected from the group consisting of ribotype 001, 003, 012, 014, 027, 036, 106, MOH 900, MOH 718, and any combination thereof.

19. The pharmaceutical composition of claim 15, wherein the PSII is a polysaccharide of Formula (I): 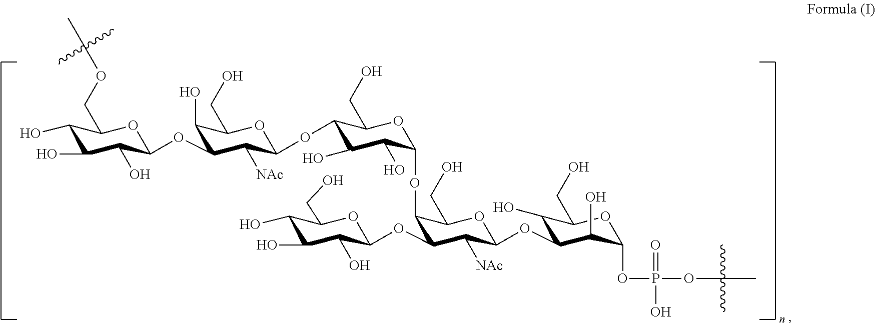
wherein n is an integer of from 1 to 100.

20. (canceled)

21. (canceled)

22. The pharmaceutical composition of claim 1, further comprising an adjuvant, wherein the adjuvant comprises aluminum hydroxide, aluminum phosphate or delta inulin microparticles.

23.-27. (canceled)

28. The pharmaceutical composition of claim 1, wherein the carrier protein is a mutant of a diphtheria toxin, wherein the carrier protein is CRM₁₉₇.

29. (canceled)

30. The pharmaceutical composition of claim 1, wherein the cell-surface polysaccharide of C. difficile is conjugated to the carrier protein by a chemical linker wherein the chemical linker comprises a thiosuccinimide or thioester.

31.-37. (canceled)

38. The pharmaceutical composition of claim 1, wherein the composition or pharmaceutical composition comprises less than 20% by weight of a polypeptide from C. difficile.

39. (canceled)

40. (canceled)

41. (canceled)

42. (canceled)

43. (canceled)

44. The pharmaceutical composition of claim 1, wherein the pharmaceutical composition comprises less than about 5% by weight of an impurity relative to the total weight of the cell-surface polysaccharide of C. difficile, wherein the impurity is a peptidoglycan, a protein, a nucleic acid, a saccharide, or a combination thereof, and wherein the impurity is a C. difficile impurity or is derived from a cell-surface extract of C. difficile.

45.-50. (canceled)

51. A method of treating an infection caused by C. difficile, the method comprising administering to a subject in need thereof a therapeutically effective amount of the pharmaceutical composition of claim 1, wherein the administering is weekly or biweekly, wherein the administering is intravenous or intramuscular, and wherein the method induces neutralization titers against the PSII antigen.

52.-74. (canceled)

75. A method of enriching for a cell-surface polysaccharide of Clostridioides difficile (C. difficile) comprising:
(a) obtaining a cell-surface extract (CSE) of one or more strains of C. difficile, and
(b) enriching for a cell-surface polysaccharide of C. difficile from the CSE, thereby obtaining an enriched cell-surface polysaccharide of C. difficile sample;
wherein the enriched cell-surface polysaccharide of C. difficile sample comprises less than about 5% by weight of a C. difficile impurity.

76.-95. (canceled)
